SEQUENCE LISTING (94 sequences)

SEQ ID NO: 1 - protein, 217 aa, synthetic construct
DDFDLDLDDFDLDLDDFDLDLDDFDLDLSSGSPKKKRKVGSTGSQLAGDIQQLLQLQQLVLVPGHHLQPPAQFLLPQAQQSQPGLLPTPNLFQLPQQTQGALLTSQPGGSGDFQPPYFPPPYQPIYPQSQDPYSHVNDPYSLNPLHAQPQPQHPGWPGQRQSQESGLLGSGLDEDWDSALFAELGYFTDTDELQLEAANETYENNFDNLDFDLDLMP

SEQ ID NO: 2 - protein, 234 aa, synthetic construct
DALDDFDLDMLGSDALDDFDLDMLGSDALDDFDLDMLGSDALDDFDLDMLINSRSSGSTGSQLAGDIQQLLQLQQLVLVPGHHLQPPAQFLLPQAQQSQPGLLPTPNLFQLPQQTQGALLTSQPGGSGDFQPPYFPPPYQPIYPQSQDPYSHVNDPYSLNPLHAQPQPQHPGWPGQRQSQESGLLGSGLDEDWDSALFAELGYFTDTDELQLEAANETYENNFDNLDFDLDLMP

SEQ ID NO: 3 - protein, 241 aa, synthetic construct
DALDDFDLDMLGSDALDDFDLDMLGSDALDDFDLDMLGSDALDDFDLDMLINSSGSPKKKRKVGSTGSQLAGDIQQLLQLQQLVLVPGHHLQPPAQFLLPQAQQSQPGLLPTPNLFQLPQQTQGALLTSQPGGSGDFQPPYFPPPYQPIYPQSQDPYSHVNDPYSLNPLHAQPQPQHPGWPGQRQSQESGLLGSGLDEDWDSALFAELGYFTDTDELQLEAANETYENNFDNLDFDLDLMP

SEQ ID NO: 4 - protein, 246 aa, synthetic construct
EIDDVIDEIISLEIDDVIDEIISLEIDDVIDEIISLEIDDVIDEIISLGGQYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 5 - protein, 208 aa, synthetic construct
DDFDLDLDDFDLDLDDFDLDLDDFDLDLSSGSDFQPPYFPPPYQPIYPQSQDPYSHVNDPYSLNPLHAQPQPQHPGWPGQRQSQESGLLGGSGTGSQLAGDIQQLLQLQQLVLVPGHHLQPPAQFLLPQAQQSQPGLLPTPNLFQLPQQTQGALLTSQPGSGLDEDWDSALFAELGYFTDTDELQLEAANETYENNFDNLDFDLDLMP

SEQ ID NO: 6 - protein, 208 aa, synthetic construct
DDFDLDLDDFDLDLDDFDLDLDDFDLDLSSGSTGSQLAGDIQQLLQLQQLVLVPGHHLQPPAQFLLPQAQQSQPGLLPTPNLFQLPQQTQGALLTSQPGGSGLDEDWDSALFAELGYFTDTDELQLEAANETYENNFDNLDFDLDLMPGSGDFQPPYFPPPYQPIYPQSQDPYSHVNDPYSLNPLHAQPQPQHPGWPGQRQSQESGLL

SEQ ID NO: 7 - protein, 200 aa, synthetic construct
DDFDLDLDDFDLDLDDFDLDLDDFDLDLSSGSPKKKRKVGSTGSQLAGDIQQLLQLQQLVLVPGHHLQPPAQFLLPQAQQSQPGLLPTPNLFQLPQQTQGALLTSQPGGSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 8 - protein, 209 aa, synthetic construct
EIDDVIDEIISLEIDDVIDEIISLEIDDVIDEIISLEIDDVIDEIISLGSTGSQLAGDIQQLLQLQQLVLVPGHHLQPPAQFLLPQAQQSQPGLLPTPNLFQLPQQTQGALLTSQPGGSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 9 - protein, 237 aa, synthetic construct
EIDDVIDEIISLEIDDVIDEIISLEIDDVIDEIISLEIDDVIDEIISLGSTGSQLAGDIQQLLQLQQLVLVPGHHLQPPAQFLLPQAQQSQPGLLPTPNLFQLPQQTQGALLTSQPGGDDFDLDLDDFDLDLDDFDLDLDDFDLDLSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 10 - protein, 255 aa, synthetic construct
EIDDVIDEIISLEIDDVIDEIISLEIDDVIDEIISLEIDDVIDEIISLGSTGSQLAGDIQQLLQLQQLVLVPGHHLQPPAQFLLPQAQQSQPGLLPTPNLFQLPQQTQGALLTSQPGGLDEDWDSALFAELGYFTDTDELQLEAANETYENNFDNLDFDLDLMPSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 11 - protein, 187 aa, synthetic construct
EIDDVIDEIISLEIDDVIDEIISLEIDDVIDEIISLEIDDVIDEIISLGSLDEDWDSALFAELGYFTDTDELQLEAANETYENNFDNLDFDLDLMPSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 12 - protein, 210 aa, synthetic construct
EIDDVIDEIISLEIDDVIDEIISLEIDDVIDEIISLEIDDVIDEIISLGSLDEDWDSALFAELGYFTDTDELQLEAANETYENNFDNLDFDLDLMPSGTGSQLAGDIQQLLQLQQLVLVPGHHLQPPAQFLLPQAQQSQPGLLPTPNLFQLPQQTQGALLTSQPGGDNLDFDLDLMPDNLDFDLDLMPDNLDFDLDLMPDNLDFDLDLMP

SEQ ID NO: 13 - protein, 189 aa, synthetic construct
EIDDVIDEIISLEIDDVIDEIISLEIDDVIDEIISLEIDDVIDEIISLGSDNLDFDLDLMPDNLDFDLDLMPDNLDFDLDLMPDNLDFDLDLMPSGVTPPPPATTNSTNPSPQGSHSAIGLSGLNPSTGPGLWWNPAPYQPGSLDEDWDSALFAELGYFTDTDELQLEAANETYENNFDNLDFDLDLMP

SEQ ID NO: 14 - protein, 141 aa, synthetic construct
EIDDVIDEIISLEIDDVIDEIISLEIDDVIDEIISLEIDDVIDEIISLGSDNLDFDLDLMPDNLDFDLDLMPDNLDFDLDLMPDNLDFDLDLMPSGVTPPPPATTNSTNPSPQGSHSAIGLSGLNPSTGPGLWWNPAPYQP

SEQ ID NO: 15 - protein, 139 aa, synthetic construct
EIDDVIDEIISLEIDDVIDEIISLEIDDVIDEIISLEIDDVIDEIISLGSVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 16 - protein, 142 aa, synthetic construct
EIDDVIDEIISLEIDDVIDEIISLEIDDVIDEIISLEIDDVIDEIISLGSDNLDFDLDLMPDNLDFDLDLMPDNLDFDLDLMPDNLDFDLDLMPGSLDEDWDSALFAELGYFTDTDELQLEAANETYENNFDNLDFDLDLMP

SEQ ID NO: 17 - protein, 139 aa, synthetic construct
DNLDFDLDLMPDNLDFDLDLMPDNLDFDLDLMPDNLDFDLDLMPSGVTPPPPATTNSTNPSPQGSHSAIGLSGLNPSTGPGLWWNPAPYQPGSLDEDWDSALFAELGYFTDTDELQLEAANETYENNFDNLDFDLDLMP

SEQ ID NO: 18 - protein, 137 aa, synthetic construct
LDEDWDSALFAELGYFTDTDELQLEAANETYENNFDNLDFDLDLMPSGVTPPPPATTNSTNPSPQGSHSAIGLSGLNPSTGPGLWWNPAPYQPDNLDFDLDLMPDNLDFDLDLMPDNLDFDLDLMPDNLDFDLDLMP

SEQ ID NO: 19 - protein, 7 aa, synthetic construct
DDFDLDL

SEQ ID NO: 20 - protein, 12 aa, synthetic construct
EIDDVIDEIISL

SEQ ID NO: 21 - protein, 11 aa, synthetic construct
DNLDFDLDLMP

SEQ ID NO: 22 - protein, 46 aa, synthetic construct
LDEDWDSALFAELGYFTDTDELQLEAANETYENNFDNLDFDLDLMP

SEQ ID NO: 23 - protein, 108 aa, synthetic construct
QYVVAAAPNLQNQQVLTGLPGVMPNIQYQVIPQFQTVDGQQLQFAATGAQVQQDGSGQIQIIPGANQQIITNRGSGGNIIAAMPNLLQQAVPLQGLANNVLSGQTQYV

SEQ ID NO: 24 - protein, 66 aa, synthetic construct
TGSQLAGDIQQLLQLQQLVLVPGHHLQPPAQFLLPQAQQSQPGLLPTPNLFQLPQQTQGALLTSQP

SEQ ID NO: 25 - protein, 57 aa, synthetic construct
DFQPPYFPPPYQPIYPQSQDPYSHVNDPYSLNPLHAQPQPQHPGWPGQRQSQESGLL

SEQ ID NO: 26 - protein, 50 aa, synthetic construct
DALDDFDLDMLGSDALDDFDLDMLGSDALDDFDLDMLGSDALDDFDLDML

SEQ ID NO: 27 - protein, 260 aa, synthetic construct
QYLPDTDDRHRIEEKRKRTYETFKSIMKKSPFSGPTDPRPPPRRIAVPSRSSASVPKPAPQPYPFTSSLSTINYDEFPTMVFPSGQISQASALAPAPPQVLPQAPAPAPAPAMVSALAQAPAPVPVLAPGPPQAVAPPAPKPTQAGEGTLSEALLQLQFDDEDLGALLGNSTDPAVFTDLASVDNSEFQQLLNQGIPVAPHTTEPMLMEYPEAITRLVTGAQRPPDPAPAPLGAPGLPNGLLSGDEDFSSIADMDFSALL

SEQ ID NO: 28 - protein, 190 aa, synthetic construct
RDSREGMFLPKPEAGSAISDVFEGREVCQPKRIRPFHPPGSPWANRPLPASLAPTPTGPVHEPVGSLTPAPVPQPLDPAPAVTPEASHLLEDPDEETSQAVKALREMADTVIPQKEEAAICGQMDLSHPPPRGHLDELTTTLESMTEDLNLDSPLTPELNEILDTFLNDECLLHAMHISTGLSIFDTSLF

SEQ ID NO: 29 - protein, 66 aa, synthetic construct; identical to SEQ ID NO: 24

SEQ ID NO: 30 - DNA, 651 bp, synthetic construct
  source 1..651: mol_type = other DNA
gatgacttcgatctcgatcttgacgatttcgacctggacttggacgattttgatctggatcttgacgactttgacttggacctttcctccggttctcctaaaaagaagaggaaggtcggatcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggagacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctggggagcggactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 31 - DNA, 702 bp, synthetic construct
  source 1..702: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggagacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctggggagcggactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 32 - DNA, 723 bp, synthetic construct
  source 1..723: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactcctccggttctcctaaaaagaagaggaaggtcggatcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggagacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctggggagcggactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 33 - DNA, 738 bp, synthetic construct
  source 1..738: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggcggacaatacgtagttgctgcggcgcctaacctgcagaaccagcaggtactcaccgggctgcccggggtcatgccgaatatacagtaccaggtaatccctcaattccaaacggtggacggtcaacagctgcagttcgccgcaacgggagcacaggtgcagcaagatggtagcgggcaaatccaaattataccgggcgcgaatcaacagattatcacaaatcgcggcagcggaggcaacattatcgcagccatgcctaatctgttgcaacaggctgtgcccctccagggactcgccaacaacgtgcttagtgggcaaacgcagtacgtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 34 - DNA, 624 bp, synthetic construct
  source 1..624: mol_type = other DNA
gatgacttcgatctcgatcttgacgatttcgacctggacttggacgattttgatctggatcttgacgactttgacttggacctttcctccggttctgacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctgggcgggtcaggaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccgggagcggactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 35 - DNA, 624 bp, synthetic construct
  source 1..624: mol_type = other DNA
gatgacttcgatctcgatcttgacgatttcgacctggacttggacgattttgatctggatcttgacgactttgacttggacctttcctccggttctacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggactggatgaagattgggattctgctctctttgctgaactcggttatttcacagacactgatgagctgcaattggaagcagcaaatgagacgtatgaaaacaattttgataatcttgattttgatttggatttgatgcctgggagcggagacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctg

SEQ ID NO: 36 - DNA, 600 bp, synthetic construct
  source 1..600: mol_type = other DNA
gatgacttcgatctcgatcttgacgatttcgacctggacttggacgattttgatctggatcttgacgactttgacttggacctttcctccggttctcctaaaaagaagaggaaggtcggatcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 37 - DNA, 627 bp, synthetic construct
  source 1..627: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggtcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 38 - DNA, 711 bp, synthetic construct
  source 1..711: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcggggatgacttcgatctcgatcttgacgatttcgacctggacttggacgattttgatctggatcttgacgactttgacttggacctttcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 39 - DNA, 765 bp, synthetic construct
  source 1..765: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcgggctggatgaagattgggattctgctctctttgctgaactcggttatttcacagacactgatgagctgcaattggaagcagcaaatgagacgtatgaaaacaattttgataatcttgattttgatttggatttgatgccttcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 40 - DNA, 561 bp, synthetic construct
  source 1..561: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcactggatgaagattgggattctgctctctttgctgaactcggttatttcacagacactgatgagctgcaattggaagcagcaaatgagacgtatgaaaacaattttgataatcttgattttgatttggatttgatgccttcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 41 - DNA, 630 bp, synthetic construct
  source 1..630: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcactggatgaagattgggattctgctctctttgctgaactcggttatttcacagacactgatgagctgcaattggaagcagcaaatgagacgtatgaaaacaattttgataatcttgattttgatttggatttgatgccttcaggaacgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagcccggcggggataatctggacttcgacttggatcttatgccagacaaccttgattttgatctggatcttatgccggataatcttgactttgatctcgatcttatgccggacaacttggacttcgatctggatctgatgcca

SEQ ID NO: 42 - DNA, 567 bp, synthetic construct
  source 1..567: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcagataatctggacttcgacttggatcttatgccagacaaccttgattttgatctggatcttatgccggataatcttgactttgatctcgatcttatgccggacaacttggacttcgatctggatctgatgccatcaggagtcacgccaccgccccctgcgacgacaaattctacaaatcccagtccgcagggtagccactccgcaataggcctgtccggactgaatccttcaactggaccgggactttggtggaatccggctccttaccaaccgggctcactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 43 - DNA, 423 bp, synthetic construct
  source 1..423: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcagataatctggacttcgacttggatcttatgccagacaaccttgattttgatctggatcttatgccggataatcttgactttgatctcgatcttatgccggacaacttggacttcgatctggatctgatgccatcaggagtcacgccaccgccccctgcgacgacaaattctacaaatcccagtccgcagggtagccactccgcaataggcctgtccggactgaatccttcaactggaccgggactttggtggaatccggctccttaccaaccg

SEQ ID NO: 44 - DNA, 417 bp, synthetic construct
  source 1..417: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 45 - DNA, 426 bp, synthetic construct
  source 1..426: mol_type = other DNA
gaaattgacgacgtgatagatgagatcataagcctggaaatagacgacgttatagacgagataatatcacttgagatagatgatgtcatcgacgaaatcatcagtctggaaatcgacgacgtaatcgatgaaattatctcattgggctcagataatctggacttcgacttggatcttatgccagacaaccttgattttgatctggatcttatgccggataatcttgactttgatctcgatcttatgccggacaacttggacttcgatctggatctgatgccaggctcactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 46 - DNA, 417 bp, synthetic construct
  source 1..417: mol_type = other DNA
gataatctggacttcgacttggatcttatgccagacaaccttgattttgatctggatcttatgccggataatcttgactttgatctcgatcttatgccggacaacttggacttcgatctggatctgatgccatcaggagtcacgccaccgccccctgcgacgacaaattctacaaatcccagtccgcagggtagccactccgcaataggcctgtccggactgaatccttcaactggaccgggactttggtggaatccggctccttaccaaccgggctcactggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 47 - DNA, 411 bp, synthetic construct
  source 1..411: mol_type = other DNA
ctggatgaagattgggattctgctctctttgctgaactcggttatttcacagacactgatgagctgcaattggaagcagcaaatgagacgtatgaaaacaattttgataatcttgattttgatttggatttgatgccttcaggagtcacgccaccgccccctgcgacgacaaattctacaaatcccagtccgcagggtagccactccgcaataggcctgtccggactgaatccttcaactggaccgggactttggtggaatccggctccttaccaaccggataatctggacttcgacttggatcttatgccagacaaccttgattttgatctggatcttatgccggataatcttgactttgatctcgatcttatgccggacaacttggacttcgatctggatctgatgcca

SEQ ID NO: 48 - DNA, 21 bp, synthetic construct
  source 1..21: mol_type = other DNA
gatgacttcgatctcgatctt

SEQ ID NO: 49 - DNA, 36 bp, synthetic construct
  source 1..36: mol_type = other DNA
gaaattgacgacgtgatagatgagatcataagcctg

SEQ ID NO: 50 - DNA, 33 bp, synthetic construct
  source 1..33: mol_type = other DNA
gataatctggacttcgacttggatcttatgcca

SEQ ID NO: 51 - DNA, 138 bp, synthetic construct
  source 1..138: mol_type = other DNA
ctggatgaagattgggattctgctctctttgctgaactcggttatttcacagacactgatgagctgcaattggaagcagcaaatgagacgtatgaaaacaattttgataatcttgattttgatttggatttgatgcct

SEQ ID NO: 52 - DNA, 324 bp, synthetic construct
  source 1..324: mol_type = other DNA
caatacgtagttgctgcggcgcctaacctgcagaaccagcaggtactcaccgggctgcccggggtcatgccgaatatacagtaccaggtaatccctcaattccaaacggtggacggtcaacagctgcagttcgccgcaacgggagcacaggtgcagcaagatggtagcgggcaaatccaaattataccgggcgcgaatcaacagattatcacaaatcgcggcagcggaggcaacattatcgcagccatgcctaatctgttgcaacaggctgtgcccctccagggactcgccaacaacgtgcttagtgggcaaacgcagtacgtc

SEQ ID NO: 53 - DNA, 198 bp, synthetic construct
  source 1..198: mol_type = other DNA
acgggcagccagctggctggagatatacagcagctcctccagctgcagcagctggtgcttgtgccaggacaccatctgcagccacctgctcagttcctgctaccgcaggcccagcagagccagccaggcctgctaccgacaccaaatctattccagctacctcagcaaacccagggagctcttctgacctcccagccc

SEQ ID NO: 54 - DNA, 171 bp, synthetic construct
  source 1..171: mol_type = other DNA
gacttccagcccccatacttccctccaccctaccagcctatctacccccagtcgcaagatccttactctcacgtcaacgatccttactctctgaatccactgcatgctcagcctcagcctcagcatccaggatggcctggccagaggcagagccaggagtctgggctcctg

SEQ ID NO: 55 - DNA, 150 bp, synthetic construct
  source 1..150: mol_type = other DNA
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctg

SEQ ID NO: 56 - DNA, 780 bp, synthetic construct
  source 1..780: mol_type = other DNA
cagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctg

SEQ ID NO: 57 - DNA, 570 bp, synthetic construct
  source 1..570: mol_type = other DNA
cgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactggatccagcgcccgcagtgactcccgaggccagtcacctgttggaggatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttt

SEQ ID NO: 58 - DNA, 198 bp, synthetic construct; identical to SEQ ID NO: 53
  source 1..198: mol_type = other DNA

SEQ ID NO: 59 - protein, 8 aa, synthetic construct
GGSGGGSG

SEQ ID NO: 60 - protein, 16 aa, synthetic construct
GGSGGGSGGGSGGGSG

SEQ ID NO: 61 - protein, 185 aa, synthetic construct
MAQAALEPGEKPYACPECGKSFSRSTNLVRHQRTHTGEKPYKCPECGKSFSECDDLARHQRTHTGEKPYKCPECGKSFSQSDNLVRHQRTHTGEKPYACPECGKSFSTSDNLVRHQRTHTGEKPYKCPECGKSFSRSDNLVRHQRTHTGEKPYKCPECGKSFSDRDHLVRHQRTHTGKKTSGQAG

SEQ ID NO: 62 - DNA, 2709 bp, synthetic construct
  source 1..2709: mol_type = other DNA
atgcccaagaagaaaaggaaagtcggaggtggaagcccgggtaaacgcaattatatcctggggttggccatcggaatcactagtgttggatatgggattattgactacgagacgcgcgatgtgatcgatgccggagtacggcttttcaaggaggcaaacgtcgagaacaatgaggggcggcggtctaaaagaggagcacgaaggctcaagaggcgaagaaggcatcggattcaacgcgtcaagaaattgctcttcgattacaatctgctgacagaccactccgagttgtccggaataaatccgtacgaggcgagagtcaaaggcctcagtcagaagctctccgaagaagagttcagcgctgcacttctccaccttgcgaaacgaagaggagtgcataacgtaaacgaagtcgaagaggatacaggcaacgagctgtctacgaaagagcagattagtaggaatagcaaggcactcgaagagaaatatgtggcggaactccaactcgaaaggctcaagaaggacggtgaagtcaggggttctataaatcggtttaaaacatccgactatgtcaaagaagcgaagcaacttcttaaggtccaaaaggcataccaccagttggatcaatcattcattgacacttacattgacctgctcgagacacgacggacttactacgaaggaccgggagaagggagtccgttcggctggaaggacattaaggaatggtatgagatgctcatgggacattgtacgtatttccccgaagaactgcgatcagtcaagtatgcttataatgcagatttgtacaacgcattgaatgatttgaataatctggtcatcacacgagacgaaaacgaaaaattggagtactacgaaaaattccagattatcgaaaatgtctttaaacagaagaagaagccgactctcaagcaaatagctaaagagattcttgtcaatgaagaagacattaaaggttaccgagttactagcactggcaaacccgagtttactaacctgaaagtgtaccacgatatcaaggacatcaccgcgcgaaaagagataatcgaaaacgccgaacttctggaccaaattgcgaaaattctgaccatttatcaatccagtgaagatatacaagaagagctgacgaaccttaactcagagcttacgcaagaggagattgagcaaataagcaatctgaagggttatacggggacccacaacctgagtctcaaagcaattaaccttatccttgacgagctttggcatacaaacgacaaccagatagctatatttaaccggctcaaactggtgccgaaaaaagtcgatctttctcagcaaaaagaaattcctacgacgctcgtggatgactttattttgtcacccgtcgtaaaacgctccttcatccaatcaatcaaagtcatcaatgcgataataaagaagtacggcctcccgaacgatataattatagagctcggttccaagcgctatgccacgcgcggtttgatgaaccttctccgatcttacttccgggtcaataacctcgacgtgaaggtaaaatcaattaatggagggttcacatcattcctccgacgaaaatggaagtttaaaaaggaacgcaacaaggggtataagcatcatgcagaggatgcacttatcatagccaacgcagactttattttcaaggagtggaaaaaattggataaagctaagaaagtcatggaaaaccagatgttcgaagagaaacaagccgaaagcatgcccgagatcgagactgagcaggaatataaagaaatcttcatcaccccgcatcaaataaagcacataaaagactttaaagattacaagtattcccatcgggtggacaaaaagcctaatcggaagctcatcaatgacactctttactccactagaaaagacgataagggcaataccctcatagttaataacctgaatggtttgtacgataaagacaacgataagctgaaaaaacttataaacaagtccccggaaaagttgcttatgtatcaccacgacccgcagacgtaccagaaacttaagcttattatggaacagtatggggatgagaaaaacccactctacaaatactacgaggaaacgggtaattatttgactaagtactcaaagaaagataatggtcctgtgataaaaaagataaagtattacgggaataaactcaacgctcacctcgacataacagatgattaccctaatagtcgaaataaagtcgttaagttgtctcttaagccgtatcgatttgacgtgtatctggacaatggcgtgtataagttcgtcacggttaaaaatcttgacgtaatcaaaaaagaaaattattatgaggtaaactccaaatgttacgaagaggcgaaaaaactcaaaaaaatatcaaatcaggccgagttcattgcgtccttttataaaaatgacctgataaagattaatggagaattgtatcgggtgataggggtgaacaatgaccttttgaatagaattgaagttaatatgatcgacataacctatcgagagtatcttgaaaatatgaacgacaagagaccgccacacatcattaaaactatcgcaagtaagacccagtcaatcaaaaaatatagcacagatatacttggcaatctttacgaggtcaagtctaagaaacaccctcagattattaagaaagggacttctcgcgcggac

SEQ ID NO: 63 - protein, 903 aa, synthetic construct
MPKKKRKVGGGSPGKRNYILGLAIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELGSKRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKGTSRAD

SEQ ID NO: 64 - RNA, 20 nt, synthetic construct
  source 1..20: mol_type = other RNA
tacgttctctatcactgata

SEQ ID NO: 65 - RNA, 20 nt, synthetic construct
  source 1..20: mol_type = other RNA
ctgatttgtattaggtacca

SEQ ID NO: 66 - RNA, 20 nt, synthetic construct
  source 1..20: mol_type = other RNA
gactttacatgaggaatttt

SEQ ID NO: 67 - RNA, 20 nt, synthetic construct
  source 1..20: mol_type = other RNA
atatgagttcaaagttatta

SEQ ID NO: 68 - RNA, 20 nt, synthetic construct
  source 1..20: mol_type = other RNA
  modified_base 1: mod_base = OTHER; note = Thymine
tgcctccctgtcctgtagta

SEQ ID NO: 69 - RNA, 20 nt, synthetic construct
  source 1..20: mol_type = other RNA
catacaatgagaattgctag

SEQ ID NO: 70 - protein, 194 aa, synthetic construct
MPKKKRKVGIHGVPAAMAERPFQCRICMRNFSRSDNLSEHIRTHTGEKPFACDICGRKFAKRCNLRCHTKIHTGSQKPFQCRICMRNFSRSDNLSEHIRTHTGEKPFACDICGRKFAKRCNLRCHTKIHTGSQKPFQCRICMRNFSRSDNLSEHIRTHTGEKPFACDICGRKFAKRCNLRCHTKIHLRQKDAAR

SEQ ID NO: 71 - protein, 192 aa, synthetic construct
MPKKKRKVAQAALEPGEKPYACPECGKSFSRKDSLVRHQRTHTGEKPYKCPECGKSFSQSGDLRRHQRTHTGEKPYKCPECGKSFSDCRDLARHQRTHTGEKPYACPECGKSFSQSSHLVRHQRTHTGEKPYKCPECGKSFSDCRDLARHQRTHTGEKPYKCPECGKSFSRSDKLVRHQRTHTGKKTSGQAG

SEQ ID NO: 72 - DNA, 18 bp, unidentified
  source 1..18: mol_type = unassigned DNA
ggggccggagccgcagtg

SEQ ID NO: 73 - protein, 189 aa, synthetic construct
CPRRKGKSHRQLSSLAKNLTHVRSVGSLSQHLITLSHIKEPTLVKSHINAQSVERVLASLTIWDIREPTLARSLTNAPNAENHSVRLITWSGIRELILVKNHMHARSAANHFPHQTILPTRELTRGRSHTSALNVESHSQSQTHSCGTSARIRGRNRTNARNAGSHSVAQITWSAINEPIPVRRQVAKL

SEQ ID NO: 74 - protein, 4 aa, synthetic construct
GGGS

SEQ ID NO: 75 - protein, 5 aa, synthetic construct
GGGGS

SEQ ID NO: 76 - protein, 4 aa, synthetic construct
GGSG

SEQ ID NO: 77-78 - skipped (2 entries)

SEQ ID NO: 79 - RNA, 76 nt, synthetic construct
  source 1..76: mol_type = other RNA
agtactctggaaacagaatctactctaacaaggcaaaatgccgtgtttatctcgtcaacttgttggcgagattttt

SEQ ID NO: 80-82 - skipped (3 entries)

SEQ ID NO: 83 - protein, 11 aa, synthetic construct
  SITE 1: note = Histidine linked to the Serine at position 21 in SEQ ID NO:92 via a peptide sequence of undefined length
HQRTHTGKKTS

SEQ ID NO: 84 - DNA, 2211 bp, Adeno-associated virus
  source 1..2211: mol_type = genomic DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 85 - DNA, 1800 bp, Adeno-associated virus
  source 1..1800: mol_type = genomic DNA
acggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 86 - DNA, 1605 bp, Adeno-associated virus
  source 1..1605: mol_type = genomic DNA
atggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 87 - protein, 736 aa, Adeno-associated virus
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 88 - protein, 599 aa, Adeno-associated virus
TAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 89 - protein, 534 aa, Adeno-associated virus
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 90 - DNA, 18 bp, unidentified
  source 1..18: mol_type = unassigned DNA
ggcgaggatgaagccgag

SEQ ID NO: 91 - protein, 18 aa, synthetic construct
  SITE 18: note = Serine linked to the Histidine at position 1 in SEQ ID NO:92 via a peptide sequence of undefined length
LEPGEKPYKCPECGKSFS

SEQ ID NO: 92 - protein, 21 aa, synthetic construct
  SITE 1: note = Histidine linked to the Serine at position 18 in SEQ ID NO:91 via a peptide sequence of undefined length
  SITE 21: note = Serine linked to the Histidine at position 1 in SEQ ID NO:83 via a peptide sequence of undefined length
HQRTHTGEKPYKCPECGKSFS

SEQ ID NO: 93 - DNA, 18 bp, synthetic construct
  source 1..18: mol_type = other DNA
cagcagcagcagcagcag

SEQ ID NO: 94 - DNA, 24 bp, synthetic construct
  source 1..24: mol_type = other DNA
cagcagcagcagcagcagcagcag

(19) United States
(12) Patent Application Publication
(10) Pub. No.: US 2024/0398985 A1
(43) Pub. Date: Dec. 5, 2024

(54) NOVEL TRANSCRIPTION FACTORS

(71) Applicant: NOVARTIS AG, Basel (CH)

(72) Inventors: Keith Everett FERGUSON, Woburn, MA (US); Stephanie Viani HROMADKA, North Reading, MA (US); Xu E. TAO, Lexington, MA (US)

(21) Appl. No.: 18/692,255

(22) PCT Filed: Sep. 15, 2022
(86) PCT No.: PCT/IB2022/058691

§ 371 Date: Mar. 14, 2024

Related U.S. Application Data
(60) Provisional application No. 63/245,084, filed on Sep. 16, 2021.

(51) Int. Cl. A61K 48/00; C12N 9/22; C12N 15/11

(52) U.S. Cl. CPC A61K 48/005; C12N 9/22; C12N 15/11; C07K 2319/81; C12N 2740/15043; C12N 2750/14143

(57) ABSTRACT

The present invention relates to novel transcription factors for modulating the expression of gene of interest, by fusing to a DNA binding domain that targets the gene of interest.

### FIELD

The present disclosure is directed to novel transcription factors for modulating the expression of a gene of interest, by fusing to a DNA binding domain that targets the gene of interest.

### BACKGROUND

Genomic alterations that result in reduced transcription or activity of one or more genes or gene products are causative agents of a wide variety of mammalian diseases. One such genomic alteration is haploinsufficiency. In haploinsufficiency, there is only one functional copy of a gene, and that single copy does not produce enough gene product to produce a wild-type phenotype. Another type of disease is caused by a genomic change in one or both copies of a gene, which alters the gene product such that the gene product exhibits reduced activity rather than elimination of activity. In yet another type of disease, genomic alterations reduce the transcription of one or both copies of a gene or reduce the stability of the transcript, so that gene product abundance produces a wild-type phenotype will be insufficient.

Numerous approaches have been attempted to treat such diseases by increasing the amount or activity of the one or more genes whose transcription or activity is reduced. One of such approach is the delivery of a wild-type copy of the target gene by Adeno-associated virus (AAV) vector to the patient to produce functional protein. Adeno-associated virus (AAV) is a small, replication-defective, non-enveloped virus that infects humans and some other primate species. Several characteristics of AAV make this virus an attractive vehicle for the delivery of therapeutic proteins by gene therapy, including, for example, that AAV is not known to cause human disease and induce a mild immune response, and that AAV vectors can infect both dividing and dormant cells without integrating into the host cell genome. AAV gene therapy vectors do have some drawbacks, however. In particular, the cloning capacity of AAV vectors is limited as a consequence of the packaging capacity of the virus DNA. The single-stranded DNA genome of wild-type AAV is approximately 4.7 kilobases (kb). In practice, AAV genomes up to about 5.0 kb appear to be fully packaged, ie, full length, into AAV virus particles. With the requirement that the nucleic acid genome in AAV vectors must have two AAV inverted terminal repeats (ITRs) of approximately 145 bases, the DNA packaging capacity of an AAV vector is such that a maximum of approximately 4.4 kb of protein coding sequence.

Due to this size restriction, large therapeutic genes, e.g., those greater than about 4.4 kb in length, are generally not suitable for use in AAV vectors. One approach to overcome the size restriction of AAV is to deliver a transcription factor that targets the promoter region of the target gene, instead of delivering the target gene itself, thereby increasing target gene transcription of the gene. Thus, there is a need for improved transcription factors, particularly small in size and strong in activity, that are suitable to be delivered by an AAV vector and can modulate the expression of any endogenous gene to help reverse the effects of a disease or disorder, in particular, a therapy with reduced immunogenicity, reduced off-target effects, increased specificity for a target gene, and/or increased therapeutic efficacy.

### SUMMARY OF THE INVENTION

In one aspect, provided here is a transcription factor comprising a DNA binding domain and at least three transcriptional activation domains (TADs), wherein the TADs may be same or different.

In some embodiments, the TADs comprises i) at least one acidic TAD and at least one Q-rich TAD, or ii) at least one acidic TAD and at least one P-rich TAD. In some embodiments, wherein the acidic TAD comprises one or more copies of a TAD selected from VP16, VP64, VP7, ATF6, TFE3, ATF6-11, or a fragment thereof. In some embodiments, the Q-rich TAD comprises one or more copies of a TAD selected from Oct2-Q, SP1-Q1, or a fragment thereof. In some embodiments, the P-rich TAD comprises one or more copies of a TAD selected from TFAP2-P, Oct2-P, or a fragment thereof.

In some embodiments, the TADs are selected from the group consisting of full-length VP64, one or multiple repeats of VP16, full-length or partial p65, full-length RTA, full-length VP7, full-length or partial TEF3, full-length or partial ATF6-11, full-length or partial ATF6-Acidic, full-length or partial SP1 Q-rich, full-length or partial Oct2 Q-rich, full-length or partial Oct2 P-rich, full-length or partial TFAP2 P-rich, active fragments of VP64, transcription-active fragments of p65, transcription-active fragments of RTA active fragments of VP7, active fragments of TEF3, active fragments of ATF6-11, active fragments of ATF6-Acidic, active fragments of SP1 Q-rich, active fragments of Oct2 Q-rich, active fragments of Oct2 P-rich, active fragments of TFAP2 P-rich, and any combinations thereof.

In some embodiments, the total length of the TADs is less than 2000aa, less than 1500aa, less than 1000aa, less than 750aa, less than 500aa, less than 300aa, less than 250aa, less than 200aa, or less than 150aa in length.

In some embodiments, the transcription factor comprises a sequence of SEQ ID NOs: 1-28, or a sequence having at least 80%, 85%, 90%, 95%, 96%, 97%, 98%, or 99% sequence identity thereto. In certain embodiments, the transcription factor comprises a sequence having any one of SEQ ID NOs: 1-18, or a sequence having at least 80%, 85%, 90%, 95%, 96%, 97%, 98%, or 99% sequence identity thereto.

In some embodiments, the nucleic acid sequence encoding the transcription factor comprises a sequence of SEQ ID Nos: 30-47 or a sequence having at least 80%, at least 85%, at least 90% or at least 95% sequence identity thereto.

In some embodiments, the DNA binding domain is linked to a transcription factor of any one of the transcription factors with or without a linker. In certain embodiments, the DNA binding domain is linked to the N-terminus of the transcription factor directly or via a linker. In certain embodiments, the DNA binding domain is linked to the C-terminus of the transcription factor directly or via a linker. In certain embodiments, the linker comprises or consists of GGSGGGSG (SEQ ID NO: 59) or GGSGGGSGGGSGGGSG (SEQ ID NO: 60).

In some embodiments, the DNA binding domain binds to a genomic region of a gene of interest. In certain embodiments, the DNA binding domain is a gRNA/Cas complex, a transcription activator-like (TAL) effector, or a zinc finger protein. In certain embodiments, the Cas molecule in the gRNA/Cas complex is or is derived from S. pyogenes Cas9, C. jejune Cas9, S. aureus Cas9, or Deltaproteobacteria (Dpb) CasX.

In some embodiments, the Cas molecule lacks one or more activities, optionally wherein the one or more activities is a cleavage activity.

In some embodiments, the Cas molecule is encoded by a nucleic acid molecule comprising fewer than 4,000 nucleotides, optionally wherein the Cas molecule is encoded by a nucleic acid molecule comprising fewer than 3,500 nucleotides, or fewer than 2,500 nucleotides. In certain embodiments, the Cas molecule is or is derived from S. aureus Cas9.

In some embodiments, the Cas molecule comprises a deletion of one or more amino acids as compared to the wild-type Cas molecule sequence.

In some embodiments, the DNA binding domain is a zinc finger protein. In some embodiments, the zinc finger protein comprises six to nine zinc finger domains. In some embodiments, the zinc finger protein binds to the promoter region of a gene of interest. In certain embodiments, the zinc finger protein binds to 6, 9, 12, 15, 18, 21, 24 or 27 bp sequence of the promoter region of the gene of interest.

In some embodiments, the Zinc finger protein comprise a sequence having at least 85%, at least 90% or at least 95% identity to SEQ ID NO: 61, SEQ ID NO: 70, SEQ ID NO: 71, or SEQ ID NO: 73.

In another aspect, the application provides a nucleic acid molecule encoding one or more components, optionally all of the components of the transcription factor of any one of the preceeding embodiments.

In another aspect, the application provides a vector comprising the nucleic acid molecule. In some embodiments, the vector comprises a first promoter operably linked to the nucleic acid sequence encoding the DNA binding domain.

In some embodiments, the vector comprises a promoter that drives transcription in a human cell.

In some embodiments, the first promoter is operable in a neuron. In certain embodiments, the neuron is GABAergic neuron or an inhibitory neuron, or an inhibitory interneuron. In certain embodiments, the neuron is a parvalbumin-positive GABAergic neuron, or somatostatin-positive GABAergic neuron, or vasoactive intestinal peptide-positive GABAergic neuron.

In some embodiments, the vector further comprising one or more of: a. a polyA sequence; b. an intron sequence; or c. an enhancer sequence.

In some embodiments, the vector further comprises a regulatory element that controls the production and/or degradation of the transcription factor.

In some embodiments, the regulatory element is a minigene linked to the transcription factor, wherein the minigene comprises a splice modulator binding sequence and wherein, in the presence of a splice modulator, said minigene undergoes splicing that results in an increased or decreased expression of the transcription factor.

In some embodiments, the regulatory element is a destabilizing domain, wherein in the presence of a small molecule that binds specifically to the destablizing domain, the expression of the transcription factor is decreased.

In some embodiments, the vector is an adenoviral vector, an adeno-associated viral (AAV) vector, or a lentiviral vector, or adenoviral vector, or herpes simplex viral vector.

In certain embodiments, the vector is an AAV vector. In certain embodiments, wherein the vector is an AAV2, AAV3, AAV4, AAV5, AAV6, AAV7, AAV8, AAV9, or AAV.rh10 vector.

In another aspect, the application provides cells comprising the vector of any one of the preceeding embodiments.

In another aspect, the application provides a method of selective expression of a transgene in a subject, comprising administering the viral vector of any of the preceeding embodiments to the subject.

In another aspect, the application provides a method of treating a disease associated with a mutation in a gene of interest, comprising administering the viral vector of any of preceeding embodiments to a subject in need thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows activity of TAD1, TAD2+ (TAD2 with three nuclear localization signal (NLS) sequences), TAD2-(TAD2 with two NLS sequences) and TAD3, compared to VPR3, for activation of TRE promoter. TADs were linked to mini Sa-Cas9 activate TRE containing promoters. Plasmid carrying sgRNA targeting TRE sequence, plasmids of mini Sa-dCas9-activator, TRE promoter-luciferase, and GPK-renilla luciferase were transfected into HEK293T cells. After two days, the luciferase activities were read. The promoter activity was calculated by normalizing firefly luciferase activity with renila luciferase activity. The relative promoter activity triggered by Sa-dCas9-activator was determined by calculating the fold change from the control guide RNA sample. 1×TRE promoter contains one TRE site; 3×TRE promoter carries three repeats of TRE site. VPR3 (Ma et al. 2018) was also linked to mini Sa-Cas9 in the same fashion, and was used a positive transcription activation factor control. The sequence of sgRNA targeting TRE is listed SEQID NO: 64.

FIG. 2 shows activity of TAD1-TAD 7, compared to VPR3 for activation of mouse SCN1A promoter. Plasmids of mini Sa-dCas9-activator (TAD1 through TAD7), mouse scn1a promoter-luciferase, gRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. Each mini Sa-dCas9-activator was transfected at 20, 6.7, and 2.2 ng/well, the results showed the dose-dependent effects. The sequence of sgRNA3 is corresponding to SEQ ID NO: 65.

FIG. 3 shows activity of TAD7-TAD10, compared to VPR3, for activation of mouse SCN1A promoter. Plasmids of mini Sa-dCas9-activator (TAD7 through TAD10), mouse scn1a promoter-luciferase, gRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. Each mini Sa-dCas9-activator was transfected at 20, 6.7, and 2.2 ng/well, the results showed the dose-dependent activations when co-transfected with active guide RNA, sgRNA3. The promoter was not activated when miniSa-dCas9 was co-transfected with an inactive guide RNA, sgRNA11. The sequences of sgRNA3 and sgRNA11 are corresponding to SEQ ID NO: 65, SEQ ID NO: 66, respectively.

FIG. 4 shows activity of TAD2 and TAD9 compared to VPR for activation of TRE protmoter and SCN1a promoter where paired with sgRNA. Mini Sa-dCas9-TADs driven by ubiquitin C promoter activate mouse scn1a promoter and TRE containing promoter when paired with active sgRNA. Various amount of mini Sa-dCas9-activators (TAD2 TAD9, and VPR), mouse scn1a promoter-luciferase or TRE containing promoter, TRE sgRNA for TRE containing promoter; sgRNA3 for mSCN1A promoter, respectively, and GPK-renilla luciferase were transfected into HEK293T cells. After two days, the luciferase activities were read. The promoter activity was calculated by normalizing firefly luciferase activity with renila luciferase activity.

FIG. 5 shows activity of TAD9, TAD11, TAD14, TAD16, compared to VP64 and full VPR, for activation of mouse SCN1A and human SCN1A promoters. Plasmid of mini Sa-dCas9-activator, mouse scn1a promoter-luciferase or human scn1a promoter-luciferase, gRNA targeting scn1a promoter, and GPK-renilla luciferase were transfected into HEK293T cells. After two days, the luciferase activities were read. The promoter activity was calculated by normalizing firefly luciferase activity with renila luciferase activity. The newly created transcription activation domains (TAD9, TAD11, TAD14, and TAD16) have comparable or higher activity than VPR (Chavez et al. 2015), and stronger activity than VP64.

FIG. 6 shows activity of TAD9, TAD11, TAD12, TAD13, TAD14, TAD15, compared to VP64 and full VPR for activation of TRE and human SCN1A promoters. Plasmid of mini Sa-dCas9-activator, TRE containing promoter or human scn1a promoter-luciferase, gRNA targeting scn1a promoter, and GPK-renilla luciferase were transfected into HEK293T cells. After two days, the luciferase activities were read. The promoter activity was calculated by normalizing firefly luciferase activity with renila luciferase activity. The newly created transcription activation domains have comparable or higher activities than VPR (Chavez et al. 2015), and have stronger activities than VP64.

FIG. 7 shows activity of TAD9, TAD11, TAD12, TAD13, TAD14, TAD15, compared to VP64 and full VPR in activation of mouse SCN1A promoter with a different active sgRNA. Plasmids of mini Sa-dCas9-activator, mouse scn1a promoter-luciferase, sgRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. The sequences of sgRNA42 is corresponding to SEQ ID NO: 67.

FIG. 8 shows activity of TAD8a, TAD9 and TAD15, compared to VP64, for activation of the TRE reporter gene in U2OS cell line. Plasmid of mini Sa-dCas9-activator, TRE containing promoter or human scn1a promoter-luciferase, gRNA targeting TRE sequence, and GPK-renilla luciferase were transfected into U2OS cells. After two days, the luciferase activities were read. The promoter activity was calculated by normalizing firefly luciferase activity with renila luciferase activity. TAD8a, TAD9 and TAD15 show higher activity than VP64, and TAD15 shows the highest activity among all.

FIG. 9 shows activity of TAD15 linked to zinc finger proteins for enhancing gene transcription. Zinc finger protein ZFP-C targeting 6 CAG repeats (SEQ ID NO: 70, “6 CAG repeats” disclosed as SEQ ID NO: 93) and zinc finger protein E2C targeting human erb2 gene promoter (SEQ ID NO: 71) were linked to TAD15. Each transcription activator were co-transfected with a reporter carrying two E2C elements (E2C 2x) or a reporter carrying 8 CAG repeats (CAG24) (SEQ ID NO: 94), and GPK-renilla luciferase were transfected into HEK293T cells. After two days, the luciferase activities were read. The promoter activity was calculated by normalizing firefly luciferase activity with renila luciferase activity. The transcription factors specifically activated promoters: E2C-TAD15 only activated E2C element containing promoter; ZFP-C-TAD15 only activated CAG repeats containing promoter.

FIG. 10 shows activity of TAD15 for activation of endogenous gene promoter. Zinc finger protein E2C (SEQ ID NO: 71) recognizes an element in human erb2 gene promoter (Beerli et al 1998). Erb2 encodes receptor protein Her2. E2C-TAD15 was tranfected into HEK293T cells. The transfected cells were identified by staining of hemagglutinin (HA), which was tagged onto E2C-TAD15. The Her2 expression levels were quantified by immunostaining of Her2 on cells surface. Cells with E2C-TAD15 (HA positive) showed more than three fold increase in Her2 level comparing to untransfected cells (HA negative).

FIG. 11 shows activity of TAD15 in activation of endogenous neuronal gene in mouse GABAergic neurons. Zinc finger protein Nav-ZF2 (SEQ ID NO: 61) targeting SCN1A promoter was linked to TAD13, TAD14, and TAD15, respectively. They were packaged into lentiviruses and used to infect cultured neurons to achieve 70-90% transduction rate. Four days later, cells were lysed and RNA were harvest. SCN1A and MAP2 transcripts were analyzed by qRT-PCR. The increase of SCN1A transcript by transcription activator is measured by fold of normalized SCN1A mRNA level over normalized SCN1A mRNA level in cells without virus.

FIG. 12 shows schematic diagram, not to scale, showing the arrangement of activation domains in each TAD molecule.

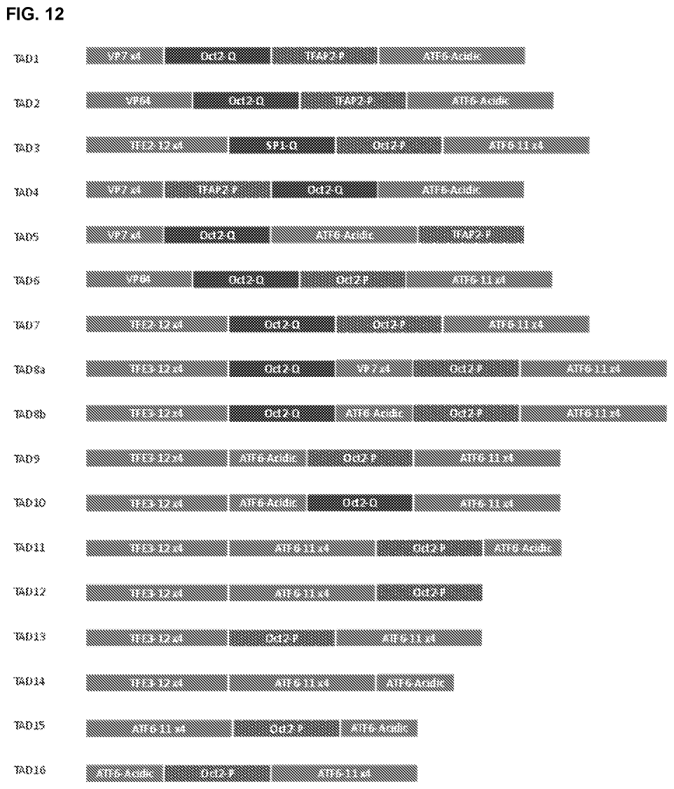

### DETAILED DESCRIPTION

### Definitions

In order that the present invention may be more readily understood, certain terms are defined throughout the detailed description. Unless defined otherwise, all technical and scientific terms used herein have the same meaning as commonly understood by those of ordinary skill in the art to which this invention pertains.

Unless stated otherwise, the following terms and phrases as used herein are intended to have the following meanings:

As used herein, a “mammal” includes any animal classified as a mammal, including, but not limited to, humans, domestic animals, farm animals, and companion animals, etc.

As used herein, the term “subject” or “patient” refers to human and non-human mammals, including but, not limited to, primates, rabbits, pigs, horses, dogs, cats, sheep, and cows. Preferably, a subject or patient is a human.

The term “a,” “an,” or “the” refers to one or to more than one of the grammatical object of the article. The term may mean “one,” “one or more,” “at least one,” or “one or more than one.” By way of example, “an element” means one element or more than one element. The term “or” means “and/or” unless otherwise stated. The term “including” or “containing” is not limiting.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “conservative sequence modifications” refers to amino acid modifications that do not significantly affect or alter the binding characteristics of the Cas9 molecule containing the amino acid sequence. Such conservative modifications include amino acid substitutions, additions and deletions. Modifications can be introduced into the Cas9 molecule or fragment described herein by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a Cas9 molecule can be replaced with other amino acid residues from the same side chain family and the altered Cas9 molecule can be tested using the functional assays described herein. Likewise, “conservative sequence modifications” refer to amino acid modifications that do not significantly affect or alter the binding characteristics of the transcription factors described herein.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to e process by which a nucleic acid sequence or a polynucleotide is transcribed from a DNA template (such as into mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “homologous,” “homology” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. The promoter or regulatory sequence may be a cis-acting element or a trans-acting element. Operably linked DNA sequences can be contiguous with each other and, e.g., where necessary to join two protein coding regions, are in the same reading frame.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” or “promoter sequence” as used herein is a DNA regulatory sequence capable of facilitating transcription (e.g., capable of causing detectable levels of transcription and/or increasing the detectable level of transcription over the level provided in the absence of the promoter) of an operatively linked coding or non-coding sequence, e.g., of a downstream (3′ direction) coding or non-coding sequence, e.g., through binding RNA polymerase. In some embodiments, the promoter sequence is bounded at its 3′ terminus by the transcription initiation site and extends upstream (5′ direction) to include the minimum number of bases or elements to initiate transcription at levels detectable above background. In some embodiments, a promoter sequence may comprise a transcription initiation site, as well as protein binding domains responsible for the binding of RNA polymerase. In addition to sequences sufficient to initiate transcription, a promoter may also include sequences of other regulatory elements that are involved in modulating transcription (e.g., enhancers, Kozak sequences and introns). Various promoters, including inducible promoters and constitutive promoters, may be used to drive the vectors disclosed herein. Examples of promoters known in the art that may be used in some embodiments, e.g., in viral vectors disclosed herein, include the CMV promoter, CBA promoter, smCBA promoter and those promoters derived from an immunoglobulin gene, SV40, or other tissue specific genes (e.g: RLBP1, RPE, VMD2). In addition, standard techniques are known in the art for creating functional promoters by mixing and matching known regulatory elements. Fragments of promoters, e.g., those that retain at least minimum number of bases or elements to initiate transcription at levels detectable above background, may also be used.

In some embodiments, a promoter can be a constitutively active promoter (i.e., a promoter that constitutively drives expression in any cell type and/or under any conditions). In other embodiments, a promoter can be a constitutively active promoter in a particular tissue context, e.g., in neurons, in cardiac cells, etc., In other embodiments, a promoter can be an inducible promoter (i.e., a promoter whose activity is controlled by an external stimulus, e.g., the presence of a particular temperature, compound, or protein). In some embodiments, a promoter may be a spatially restricted promoter that can drive activity or not depending on the physical context in which the promoter is found. Non-limiting examples of spatially restricted promoters include tissue specific promoter, cell type specific promoter, etc. In some embodiments, a promoter may be a temporally restricted promoter that drives expression depending on the temporal context in which the promoter is found. For example, a temporally restricted promoter may drive expression only at specific stages of embryonic development or during specific stages of a biological process. Non-limiting examples of temporally restricted promoters include hair follicle cycle promoters in mice.

In some embodiments, the promoter is tissue-specific such that, in a multi-cellular organism, the promoter drives expression only in a subset of specific cells. For example, tissue-specific promoters include, but are not limited to, neuron-specific promoters, adipocyte-specific promoters, cardiomyocyte-specific promoters, smooth muscle-specific promoters, photoreceptor-specific promoters, etc., A neuron-specific promoter refers to a promoter that, when administered e.g., peripherally, directly into the central nervous system (CNS), or delivered to neuronal cells, including in vitro, ex vivo, or in vivo, preferentially drives or regulates expression of an operatively-linked heterologous nucleic acid, e.g., one encoding a protein or peptide or shRNA of interest, in neurons as compared to expression in non-neuronal cells. As used herein, the terms “treat”, “treatment” and “treating” refer to a partial or complete reduction or amelioration of the progression, severity, and/or duration of a seizure disorder, or the amelioration of one or more symptoms (preferably, one or more discernible symptoms) of a seizure disorder resulting from the administration of one or more therapies.

The terms “DNA regulatory sequences,” “control elements,” and “regulatory elements,” used interchangeably herein, refer to transcriptional and translational control sequences, such as promoters, enhancers, silencers, polyadenylation signals, terminators, protein degradation signals, and the like, that provide for and/or regulate transcription of a non-coding sequence (e.g., a short hairpin RNA) or a coding sequence (e.g., PGRN) and/or regulate translation of an encoded polypeptide.

In specific embodiments, the terms “treat”, “treatment” and “treating” refer to the amelioration of at least one measurable physical parameter of a seizure disorder, as well as parameters not necessarily discernible by the patient. In other embodiments the terms “treat”, “treatment” and “treating”-refer to the inhibition of the progression of a seizure disorder, e.g., stabilization of a discernible symptom, physiologically by, e.g., stabilization of a physical parameter, or both.

A “therapeutic” as used herein means a treatment. A therapeutic effect is obtained by partial or complete reduction, suppression, remission, or eradication of a disease state or symptom.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to a molecule that preferentially recognizes and binds a binding partner (e.g., a protein or nucleic acid) over other molecules present in a sample.

Ranges: throughout this disclosure, various aspects can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range. All specified ranges also include the endpoints unless otherwise stated.

The terms “gene editing system” or “genome editing system” refer to a system of one or more molecules comprising at least a nuclease (or nuclease domain) and a programmable nucleotide binding domain, which are necessary and sufficient to direct and effect modification (e.g., single or double-strand break) of nucleic acid at a target sequence by the nuclease (or nuclease domain). In embodiments, the gene editing system is a CRISPR system. In embodiments, the gene editing system is a zinc finger nuclease (ZFN) system. In embodiments, the gene editing system is a TALEN system. In embodiments, the gene editing system is a meganuclease system. In embodiments, the gene editing system modifies the expression of a first target, i.e., SCN1A. In embodiments, the gene editing system further comprises a template nucleic acid as described herein. In embodiments, one or more of the components of the gene editing system may be introduced into cells as nucleic acid encoding said component or components. Without being bound by theory, upon expression of said component or component, the gene editing system is constituted, e.g., in the cell.

The term “sequence-specific transcription regulation system” and the like refers to a system of one or more molecules comprising at least a component which binds to a DNA sequence specifically and a component which is capable of modulating, e.g., increasing or decreasing, transcription. In exemplary systems, the sequence-specific transcription regulation system is derived from a genome editing system, for example, where one or more activities, e.g., one or more nuclease activities, of the genome editing system is altered (e.g., abolished). In exemplary sequence-specific transcription regulation systems, the component which binds to the DNA sequence specifically is a zinc finger molecule. In exemplary sequence-specific transcription regulation systems, the component which binds to the DNA sequence specifically is a TALEN molecule. In exemplary sequence-specific transcription regulation systems, the component which binds to the DNA sequence specifically is a meganuclease. It will be recognized, however, that any molecule which can be engineered to bind a target sequence in a sequence specific molecule will be useful as the component which binds to DNA sequence specifically.

The terms “CRISPR system,” “Cas system” or “CRISPR/Cas system” refer to a set of molecules comprising an RNA-guided nuclease or other effector molecule (or a molecule derived from such molecule) and a guide RNA molecule that together are necessary and sufficient to direct and effect modification of nucleic acid at a target sequence by the RNA-guided nuclease or other effector molecule. In one embodiment, a CRISPR system comprises a guide RNA molecule and a Cas protein, e.g., a Cas9 protein. Such systems comprising a Cas9 or modified Cas9 molecule are referred to herein as “Cas9 systems” or “CRISPR/Cas9 systems.” In one example, the guide RNA molecule and Cas molecule may be complexed, to form a ribonuclear protein (RNP) complex. In some examples, the effector molecule may be modified to alter one or more activities of the wild-type molecule, such as, for example, one or more nuclease activities.

The terms “guide RNA,” “guide RNA molecule,” “gRNA molecule” or “gRNA” are used interchangeably, and refer to a set of nucleic acid molecules that promote the specific directing of an RNA-guided nuclease or other effector molecule (typically in complex with the gRNA molecule) to a target sequence. A gRNA molecule may have a number of domains, as described more fully below. In some embodiments, a gRNA molecule comprises a targeting domain and interacts with a Cas molecule, such as Cas9 or with another RNA-guided endonuclease such as Cpf1. In some embodiments, a gRNA molecule comprises a crRNA domain (comprising a targeting domain) and a tracr, e.g., for interacting with a Cas molecule such as Cas9. In some embodiments, directing of nuclease binding is accomplished through hybridization of a portion of the gRNA to DNA (e.g., through the gRNA targeting domain), and by binding of a portion of the gRNA molecule to the RNA-guided nuclease or other effector molecule (e.g., through at least the gRNA tracr). In embodiments, the crRNA and the tracr are provided on a single contiguous polynucleotide molecule, referred to herein as a “single guide RNA,” “sgRNA,” or “single-molecule DNA-targeting RNA” and the like. In other embodiments, the crRNA and tracr are provided on separate polynucleotide molecules, which are themselves capable of association, usually through hybridization, referred to herein as a “dual guide RNA,” “dgRNA,” or “double-molecule DNA-targeting RNA” and the like. In some embodiments of dgRNAs, the crRNA and tracr are linked by a nonnucleotide chemical linker.

The term “targeting domain” as used herein in connection with a gRNA, is the portion of the gRNA molecule that recognizes, e.g., is complementary to, a target sequence, e.g., a target sequence within the nucleic acid of a cell.

The term “crRNA” as used herein in connection with a gRNA molecule, is a portion of the gRNA molecule that comprises a targeting domain. In embodiments, the crRNA comprises a region that interacts with a tracr to form a flagpole region. In some embodiments, a crRNA can interact directly with an RNA-guided endonuclease, such as a Cas protein (e.g., Cpf1), without a tracr RNA.

The term “target sequence” refers to a sequence of nucleic acids complementary, e.g., fully complementary, to a gRNA targeting domain and also refers to the sequence of nucleic acids recognized by (e.g., bound by) the DNA-binding component of a sequence-specific transcription regulation system, for example, the sequence recognized by a zinc finger motif or a sequence recognized by a TALEN or homing endonuclease. In embodiments, the target sequence is disposed on genomic DNA. In an embodiment, particularly for embodiments comprising an RNA-guided endonuclease system, the target sequence is adjacent to (either on the same strand or on the complementary strand of DNA) a protospacer adjacent motif (PAM) sequence recognized by a protein having nuclease or other effector activity, e.g., a PAM sequence recognized by Cas9. The PAM sequence and length may depend on the Cas9 protein used. Non-limiting examples of PAM sequences include 5′-NGG-3′, 5′-NGGNG-3′, 5′-NG-3′, 5′-NAAAAN-3′, 5′-NNAAAAW-3′, 5′-NNNNACA-3′, 5′-GNNNCNNA-3′, 5′-NNGRRT-3′, 5′-NNGRRN-3′, and 5′-NNNNGATT-3′ where N represents any nucleotide, R represents A or G, and W represents A or T.

In embodiments, the target sequence falls within the exon sequences of SCN1A. In other embodiments, the target sequence falls within the intron sequences of SCN1A. In yet other embodiments, the target sequence lies in nucleic acid regions adjacent to the SCN1A gene. In still further embodiments, the target sequence overlaps with one or more of an intronic sequence of SCN1A, an exonic sequence of SCN1A, and a nucleic acid region adjacent to the SCN1A.

The term “flagpole” as used herein in connection with a gRNA molecule, refers to the portion of the gRNA where the crRNA and the tracr bind to, or hybridize to, one another.

The term “tracr” or “tracrRNA” as used herein in connection with a gRNA molecule refers to the portion of the gRNA that binds to a nuclease or other effector molecule. In embodiments, the tracr comprises nucleic acid sequence that binds specifically to Cas9. In embodiments, the tracr comprises nucleic acid sequence that forms part of the flagpole.

The term “Cas” refers to an RNA-guided nuclease of the CRISPR system that together with a guide RNA molecule are necessary and sufficient to direct and effect modification of nucleic acid at a target sequence. One non-limiting example is a Cas molecule from the Type II CRISPR system, e.g., a Cas9 molecule. Another non-limiting example is a Cas molecule is from a Type V CRISPR system, e.g., a Cpf1 molecule.

The terms “Cas9” and “Cas9 molecule” refer to an enzyme from a bacterial Type II CRISPR/Cas system responsible for DNA cleavage. In embodiments, Cas9 also includes wild-type protein, mutant protein, variant protein, including non-catalytic protein, and functional fragments thereof. Non-limiting examples of Cas9 sequences are known in the art and provided herein. In some embodiments, Cas9 refers to a Cas9 sequence that comprises at least about 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to any Cas9 sequence, e.g., wild-type, mutant, variant, non-catalytic, or functional fragment thereof, known in the art or disclosed herein. In one preferred embodiment, the Cas9 molecule is obtained from Staphylococcus aureus. In another embodiment, the Cas9 molecule is a mutant protein with no detectable nuclease activity. In yet another embodiment, the Cas9 molecule is a mutant protein that is fused to transcription activators.

The terms “Cpf1” and “Cpf1 molecule” refer to an enzyme from a bacterial Type V CRISPR/Cas system responsible for DNA cleavage. In embodiments, Cpf1 also includes wild-type protein, mutant protein, variant protein, including non-catalytic protein, and functional fragments thereof. Non-limiting examples of Cpf1 sequences are known in the art. In some embodiments, Cpf1 refers to a Cpf1 sequence that comprises at least about 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to any Cpf1 sequence, e.g., wild-type, mutant, variant, non-catalytic, or functional fragment thereof, known in the art. Unlike other Cas proteins (e.g. Cas9) Cpf1 does not require tracrRNA for activity and is capable of binding and cleaving genomic target sequences with only a crRNA polynucleotide. Therefore, in some embodiments that utilize Cpf1 to edit target sequences, the gRNA may lack a tracrRNA moiety. The term “complementary” as used in connection with nucleic acid, refers to the pairing of bases, A with T or U, and G with C. The term complementary can also refer to nucleic acid molecules that are completely complementary, that is, form A to T or U pairs and G to C pairs across the entire reference sequence, as well as molecules that are at least about 80%, 85%, 90%, 95%, or 99% complementary.

The term “gene” or “gene sequence” is meant to refer to a genetic sequence, e.g., a nucleic acid sequence. The term “gene” is intended to encompass a complete gene sequence or a partial gene sequence. The term “gene” refers to a sequence that encodes a protein or polypeptide or a sequence that does not encode a protein or polypeptide, e.g., a regulatory sequence, leader sequence, signal sequence, intron, or other non-protein coding sequence.

The term “intron” refers to nucleic acid sequence within a gene which is noncoding for the protein expressed from said gene. Intronic sequence may be transcribed from DNA into RNA, but may be removed before the protein is expressed

The term “exon” refers to nucleic acid sequence within a gene which encodes a protein expressed from said gene.

The term “intron-exon junction,” when used in connection with a gene editing system or sequence-specific transcription regulation system or gRNA molecule, refers to a sequence which includes nucleotides of an exon and nucleotides of an intron. In exemplary embodiments, an intron-exon junction is a gRNA target sequence, whereby, when recognized by a CRISPR system comprising a gRNA comprising a targeting domain complementary to the intron-exon junction target sequence, said CRISPR system modifies at or near the target sequence between two nucleotides of an intron. In other exemplary embodiments, an intron-exon junction is a gRNA target sequence, whereby, when recognized by a CRISPR system comprising a gRNA comprising a targeting domain complementary to the intron-exon junction target sequence, said CRISPR system modifies at or near the target sequence between two nucleotides of an exon. In other exemplary embodiments, an intron-exon junction is a gRNA target sequence, whereby, when recognized by a CRISPR system comprising a gRNA comprising a targeting domain complementary to the intron-exon junction target sequence, said CRISPR system modifies at or near the target sequence between a nucleotide of an exon and a nucleotide of an intron.

The term “AAV” is an abbreviation for adeno-associated virus, and may be used to refer to the virus itself or a derivative thereof. The term covers all serotypes, subtypes, and both naturally occurring and recombinant forms, except where required otherwise. The abbreviation “rAAV” refers to recombinant adeno-associated virus, also referred to as a recombinant AAV vector (or “rAAV vector”). The term “AAV” includes AAV1, AAV2, AAV3, AAV4, AAV5, AAV6, AAV7, AAV8, AAV9, AAV10, AAV11, AAV12, rhIO, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). An rAAV vector may either be single-stranded (ssAAV) or self-complementary (scAAV). An “AAV virus” or “AAV viral particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV vector particle” or simply an “rAAV particle”. Thus, production of rAAV particle necessarily includes production of rAAV vector, as such a vector is contained within an rAAV particle.

As used herein, the terms “treat”, “treatment”, “therapy” and the like refer to alleviating, delaying or slowing the progression, prophylaxis, attenuation, reducing the effects or symptoms, preventing onset, inhibiting, or ameliorating the onset of the diseases or disorders. The methods of the present disclosure may be used with any mammal. Exemplary mammals include, but are not limited to rats, cats, dogs, horses, cows, sheep, pigs, and more preferably humans. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, a therapeutic may be administered to a subject at risk of developing a particular disease, or to a subject reporting one or more of the physiological symptoms of a disease, even though a diagnosis of this disease may not have been made. The methods of the present disclosure may be used with any mammal. In some cases, the treatment can result in a decrease or cessation of symptoms (e.g., a reduction in the frequency, duration and/or severity of seizures). A prophylactic effect includes delaying or eliminating the appearance of a disease or condition, delaying or eliminating the onset of symptoms of a disease or condition, slowing, halting, or reversing the progression of a disease or condition, or any combination thereof.

A “fragment” of a nucleotide or peptide sequence refers to a sequence that is shorter than a reference or “full-length” sequence.

A “variant” of a molecule refers to allelic variations of such sequences, that is, a sequence substantially similar in structure and biological activity to either the entire molecule, or to a fragment thereof.

A “functional fragment” of a DNA or protein sequence refers to a fragment that retains a biological activity (either functional or structural) that is substantially similar to a biological activity of the full-length DNA or protein sequence. A biological activity of a DNA sequence can be its ability to influence expression in a manner known to be attributed to the full-length sequence.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

In general, “sequence identity” or “sequence homology”, which can be used interchangeably, refer to an exact nucleotide-to-nucleotide or amino acid-to-amino acid correspondence of two polynucleotides or polypeptide sequences, respectively. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et ah, J. Mol. Biol. 215:403-410 (1990); Karlin and Altschul, Proc. Natl. Acad. Sci. USA 90:5873-5877 (1993); and Altschul et ah, Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17:149-163 (1993). High sequence identity generally includes ranges of sequence identity of approximately 80% to 100% and integer values there between. As used herein, “engineered” with reference to a protein refers to a non-naturally occurring protein, including, but not limited to, a protein that is derived from a naturally occurring protein, or where a naturally occurring protein has been modified or reprogrammed to have a certain property.

As used herein, “synthetic” and “artificial” are used interchangeably to refer to a protein or a domain thereof that has low sequence identity (e.g., less than 50% sequence identity) to a naturally occurring human protein.

As used herein, an “transcription factor” or “TF” refers to as a DNA binding protein or a non-naturally occurring transcription modulator that has been modified or reprogrammed to bind to a specific target binding site and/or to include a modified or replaced transcription effector domain.

As used herein, a “DNA binding domain” can be used to refer to one or more DNA binding motifs, such as a Cas molecule, a transcription activator-like (TAL) effector, or a zinc finger protein or a basic helix-loop-helix (bHLH) motif, individually or collectively as part of a DNA binding protein.

The terms “transcription activation domain”, “transcriptional activation domain”, “transactivation domain”, “trans-activating domain” and “TAD” are used interchangeably herein and refer to a domain of a protein which in conjunction with a DNA binding domain can activate transcription from a promoter by contacting transcriptional machinery (e.g., general transcription factors and/or RNA polymerase) either directly or through other proteins known as co-activators. “Q-rich TAD” refers to glutamine rich transactivation domain. “P-rich TAD” refers to proline rich transactivation domain. “Acidic TAD” refers to transactivation domains that are rich in acidic amino acids.

### Transcription Factors

Transcription factors (TFs) are proteins that bind specific sequences in the genome and control the expression of genes. The present disclosure provides novel engineered transcription factors comprising a DNA binding domain (DBD) and at least three transcriptional activation domains (TADs). In some embodiments, the TADs may be the same. In some embodiments, the TADs may be different.

Transcriptional activation domains found within various proteins have been grouped into categories based upon similar structural features. Types of transcription activation domains which can be used in the present application include acidic transcription activation domains, proline-rich (P-rich) transcription activation domains, and glutamine-rich (Q-rich) transcription activation domains, and/or fragments thereof. Examples of acidic transcriptional activation domains include VP16, VP64, VP7, ATF6, TFE3, ATF6-11, or a fragment thereof. Examples of proline-rich activation domains include TFAP2-P, Oct2-P, or a fragment thereof. Examples of glutamine-rich activation domains include amino acid residues Oct2-Q, SP1-Q1, or a fragment thereof. The amino acid sequences of each of the above-described regions are disclosed in Table 1 of the application.

In certain embodiments, the TADs comprises i) at least one acidic TAD and at least one Q-rich TAD, or ii) at least one acidic TAD and at least one P-rich TAD.

In some embodiments, wherein the acidic TAD comprises one or more copies of a TAD selected from VP16, VP64, VP7, ATF6, TFE3, ATF6-11, or a fragment thereof. In some embodiments, the Q-rich TAD comprises one or more copies of a TAD selected from Oct2-Q, SP1-Q1, or a fragment thereof. In some embodiments, the P-rich TAD comprises one or more copies of a TAD selected from TFAP2-P, Oct2-P, or a fragment thereof.

In some embodiments, the TADs are selected from the group consisting of full-length VP64, one or multiple repeats of VP16, full-length or partial p65, full-length RTA, full-length VP7, full-length or partial TEF3, full-length or partial ATF6-11, full-length or partial ATF6-Acidic, full-length or partial SP1 Q-rich, full-length or partial Oct2 Q-rich, full-length or partial Oct2 P-rich, full-length or partial TFAP2 P-rich, active fragments of VP64, transcription-active fragments of p65, transcription-active fragments of RTA active fragments of VP7, active fragments of TEF3, active fragments of ATF6-11, active fragments of ATF6-Acidic, active fragments of SP1 Q-rich, active fragments of Oct2 Q-rich, active fragments of Oct2 P-rich, active fragments of TFAP2 P-rich, and any combinations thereof.

In some embodiments, the total length of the at least three TADs is less than 2000aa, less than 1500aa, less than 1000aa, less than 750aa, less than 500aa, less than 300aa, less than 250aa, less than 200aa, or less than 150aa.

In some embodiments, the transcription factor comprises a sequence of SEQ ID NOs: 1-28, or a sequence having at least 90% or at least 95% sequence identity thereto. In certain embodiments, the transcription factor comprises a sequence having any one of SEQ ID NOs: 1-18.

In some embodiments, the nucleic acid sequence encoding the transcription factor comprises a sequence of SEQ ID Nos: 30-47 or a sequence having at least 80%, at least 85%, at least 90% or at least 95% sequence identity thereof.

In some embodiments, the DNA binding domain is linked to a transcription factor of any one of the transcription factors with or without a linker. In certain embodiments, the DNA binding domain is linked to the N-terminus of the transcription factor directly or via a linker. In certain embodiments, the DNA binding domain is linked to the C-terminus of the transcription factor directly or via a linker.

In some embodiments, the linker has 1, 2, 3, 4, 5, 6, 7, 8, 9, 10, 11, 12, 13, 14, 15, 16, 17, 18, 19, 20, 25, 30, 35, 40, 50, 60, 70, 75, 80, 90, or 100 amino acids, or from 1-5, 1-10, 1-20, 1-30, 1-40, 1-50, 1-75, 1-100, 5-10, 5-20, 5-30, 5-40, 5-50, 5-75, 5-100, 10-20, 10-30, 10-40, 10-50, 10-75, 10-100, 20-30, 20-40, 20-50, 20-75, or 20-100 amino acids.

Suitable linkers can be flexible, cleavable, non-cleavable, hydrophilic and/or hydrophobic. In centain embodiments, a linker comprises a plurality of glycine and/or serine residues. Examples of glycine/serine peptide linkers include [GS]n, [GGGS]n (SEQ ID NO: 74), [GGGGS]n (SEQ ID NO: 75), [GGSG]n (SEQ ID NO: 76), wherein n is an integer equal to or greater than 1. In certain embodiments, the linker comprises or consists of GGSGGGSG (SEQ ID NO: 59) or GGSGGGSGGGSGGGSG (SEQ ID NO: 60).

| | | |
|---|---|---|
| SEQ ID | DDFDLDLDDFDLDLDDFDLD | TAD-1 (amino |
| NO: 1 | LDDFDLDLSSGSPKKKRKVG | acid sequence) |
| | STGSQLAGDIQQLLQLQQLV | |
| | LVPGHHLQPPAQFLLPQAQQ | |
| | SQPGLLPTPNLFQLPQQTQG | |
| | ALLTSQPGGSGDFQPPYFPP | |
| | PYQPIYPQSQDPYSHVNDPY | |
| | SLNPLHAQPQPQHPGWPGQR | |
| | QSQESGLLGSGLDEDWDSAL | |
| | FAELGYFTDTDELQLEAANE | |
| | TYENNFDNLDFDLDLMP | |
| SEQ ID | DALDDFDLDMLGSDALDDFD | TAD-2 Minus |
| NO: 2 | LDMLGSDALDDFDLDMLGSD | (amino acid |
| | ALDDFDLDMLINSRSSGSTG | sequence) |
| | SQLAGDIQQLLQLQQLVLVP | |
| | GHHLQPPAQFLLPQAQQSQP | |
| | GLLPTPNLFQLPQQTQGALL | |
| | TSQPGGSGDFQPPYFPPPYQ | |
| | PIYPQSQDPYSHVNDPYSLN | |
| | PLHAQPQPQHPGWPGQRQSQ | |
| | ESGLLGSGLDEDWDSALFAE | |
| | LGYFTDTDELQLEAANETYE | |
| | NNFDNLDFDLDLMP | |
| SEQ ID | DALDDFDLDMLGSDALDDFD | TAD-2 Plus |
| NO: 3 | LDMLGSDALDDFDLDMLGSD | (amino acid |
| | ALDDFDLDMLINSSGSPKKK | sequence) |
| | RKVGSTGSQLAGDIQQLLQL | |
| | QQLVLVPGHHLQPPAQFLLP | |
| | QAQQSQPGLLPTPNLFQLPQ | |
| | QTQGALLTSQPGGSGDFQPP | |
| | YFPPPYQPIYPQSQDPYSHV | |
| | NDPYSLNPLHAQPQPQHPGW | |
| | PGQRQSQESGLLGSGLDEDW | |
| | DSALFAELGYFTDTDELQLE | |
| | AANETYENNFDNLDFDLDLM | |
| | P | |
| SEQ ID | EIDDVIDEIISLEIDDVIDE | TAD-3 (amino |
| NO: 4 | IISLEIDDVIDEIISLEIDD | acid sequence) |
| | VIDEIISLGGQYVVAAAPNL | |
| | QNQQVLTGLPGVMPNIQYQV | |
| | IPQFQTVDGQQLQFAATGAQ | |
| | VQQDGSGQIQIIPGANQQII | |
| | TNRGSGGNIIAAMPNLLQQA | |
| | VPLQGLANNVLSGQTQYVTP | |
| | PPPATTNSTNPSPQGSHSAI | |
| | GLSGLNPSTGPGLWWNPAPY | |
| | QPDNLDFDLDLMPDNLDFDL | |
| | DLMPDNLDFDLDLMPDNLDF | |
| | DLDLMP | |
| SEQ ID | DDFDLDLDDFDLDLDDFDLD | TAD-4 (amino |
| NO: 5 | LDDFDLDLSSGSDFQPPYFP | acid sequence) |
| | PPYQPIYPQSQDPYSHVNDP | |
| | YSLNPLHAQPQPQHPGWPGQ | |
| | RQSQESGLLGGSGTGSQLAG | |
| | DIQQLLQLQQLVLVPGHHLQ | |
| | PPAQFLLPQAQQSQPGLLPT | |
| | PNLFQLPQQTQGALLTSQPG | |
| | SGLDEDWDSALFAELGYFTD | |
| | TDELQLEAANETYENNFDNL | |
| | DFDLDLMP | |
| SEQ ID | DDFDLDLDDFDLDLDDFDLD | TAD-5 (amino |
| NO: 6 | LDDFDLDLSSGSTGSQLAGD | acid sequence) |
| | IQQLLQLQQLVLVPGHHLQP | |
| | PAQFLLPQAQQSQPGLLPTP | |
| | NLFQLPQQTQGALLTSQPGG | |
| | SGLDEDWDSALFAELGYFTD | |
| | TDELQLEAANETYENNFDNL | |
| | DFDLDLMPGSGDFQPPYFPP | |
| | PYQPIYPQSQDPYSHVNDPY | |
| | SLNPLHAQPQPQHPGWPGQR | |
| | QSQESGLL | |
| SEQ ID | DDFDLDLDDFDLDLDDFDLD | TAD-6 (amino |
| NO: 7 | LDDFDLDLSSGSPKKKRKVG | acid sequence) |
| | STGSQLAGDIQQLLQLQQLV | |
| | LVPGHHLQPPAQFLLPQAQQ | |
| | SQPGLLPTPNLFQLPQQTQG | |
| | ALLTSQPGGSGVTPPPPATT | |
| | NSTNPSPQGSHSAIGLSGLN | |
| | PSTGPGLWWNPAPYQPDNLD | |
| | FDLDLMPDNLDFDLDLMPDN | |
| | LDFDLDLMPDNLDFDLDLMP | |
| SEQ ID | EIDDVIDEIISLEIDDVIDE | TAD-7 (amino |
| NO: 8 | IISLEIDDVIDEIISLEIDD | acid sequence) |
| | VIDEIISLGSTGSQLAGDIQ | |
| | QLLQLQQLVLVPGHHLQPPA | |
| | QFLLPQAQQSQPGLLPTPNL | |
| | FQLPQQTQGALLTSQPGGSG | |
| | VTPPPPATTNSTNPSPQGSH | |
| | SAIGLSGLNPSTGPGLWWNP | |
| | APYQPDNLDFDLDLMPDNLD | |
| | FDLDLMPDNLDFDLDLMPDN | |
| | LDFDLDLMP | |
| SEQ ID | EIDDVIDEIISLEIDDVIDE | TAD-8a (amino |
| NO: 9 | IISLEIDDVIDEIISLEIDD | acid sequence) |
| | VIDEIISLGSTGSQLAGDIQ | |
| | QLLQLQQLVLVPGHHLQPPA | |
| | QFLLPQAQQSQPGLLPTPNL | |
| | FQLPQQTQGALLTSQPGGDD | |
| | FDLDLDDFDLDLDDFDLDLD | |
| | DFDLDLSGVTPPPPATTNST | |
| | NPSPQGSHSAIGLSGLNPST | |
| | GPGLWWNPAPYQPDNLDFDL | |
| | DLMPDNLDFDLDLMPDNLDF | |
| | DLDLMPDNLDFDLDLMP | |
| SEQ ID | EIDDVIDEIISLEIDDVIDE | TAD-8b (amino |
| NO: | IISLEIDDVIDEIISLEIDD | acid sequence) |
| 10 | VIDEIISLGSTGSQLAGDIQ | |
| | QLLQLQQLVLVPGHHLQPPA | |
| | QFLLPQAQQSQPGLLPTPNL | |
| | FQLPQQTQGALLTSQPGGLD | |
| | EDWDSALFAELGYFTDTDEL | |
| | QLEAANETYENNFDNLDFDL | |
| | DLMPSGVTPPPPATTNSTNP | |
| | SPQGSHSAIGLSGLNPSTGP | |
| | GLWWNPAPYQPDNLDFDLDL | |
| | MPDNLDFDLDLMPDNLDFDL | |
| | DLMPDNLDFDLDLMP | |
| SEQ ID | EIDDVIDEIISLEIDDVIDE | TAD-9 (amino |
| NO: | IISLEIDDVIDEIISLEIDD | acid sequence) |
| 11 | VIDEIISLGSLDEDWDSALF | |
| | AELGYFTDTDELQLEAANET | |
| | YENNFDNLDFDLDLMPSGVT | |
| | PPPPATTNSTNPSPQGSHSA | |
| | IGLSGLNPSTGPGLWWNPAP | |
| | YQPDNLDFDLDLMPDNLDFD | |
| | LDLMPDNLDFDLDLMPDNLD | |
| | FDLDLMP | |
| SEQ ID | EIDDVIDEIISLEIDDVIDE | TAD-10 (amino |
| NO: | IISLEIDDVIDEIISLEIDD | acid sequence) |
| 12 | VIDEIISLGSLDEDWDSALF | |
| | AELGYFTDTDELQLEAANET | |
| | YENNFDNLDFDLDLMPSGTG | |
| | SQLAGDIQQLLQLQQLVLVP | |
| | GHHLQPPAQFLLPQAQQSQP | |
| | GLLPTPNLFQLPQQTQGALL | |
| | TSQPGGDNLDFDLDLMPDNL | |
| | DFDLDLMPDNLDFDLDLMPD | |
| | NLDFDLDLMP | |
| SEQ ID | EIDDVIDEIISLEIDDVIDE | TAD-11 (amino |
| NO: | IISLEIDDVIDEIISLEIDD | acid sequence) |
| 13 | VIDEIISLGSDNLDFDLDLM | |
| | PDNLDFDLDLMPDNLDFDLD | |
| | LMPDNLDFDLDLMPSGVTPP | |
| | PPATTNSTNPSPQGSHSAIG | |
| | LSGLNPSTGPGLWWNPAPYQ | |
| | PGSLDEDWDSALFAELGYFT | |
| | DTDELQLEAANETYENNFDN | |
| | LDFDLDLMP | |
| SEQ ID | EIDDVIDEIISLEIDDVIDE | TAD-12 (amino |
| NO: | IISLEIDDVIDEIISLEIDD | acid sequence) |
| 14 | VIDEIISLGSDNLDFDLDLM | |
| | PDNLDFDLDLMPDNLDFDLD | |
| | LMPDNLDFDLDLMPSGVTPP | |
| | PPATTNSTNPSPQGSHSAIG | |
| | LSGLNPSTGPGLWWNPAPYQ | |
| | P | |
| SEQ ID | EIDDVIDEIISLEIDDVIDE | TAD-13 (amino |
| NO: | IISLEIDDVIDEIISLEIDD | acid sequence) |
| 15 | VIDEIISLGSVTPPPPATTN | |
| | STNPSPQGSHSAIGLSGLNP | |
| | STGPGLWWNPAPYQPDNLDF | |
| | DLDLMPDNLDFDLDLMPDNL | |
| | DFDLDLMPDNLDFDLDLMP | |
| SEQ ID | EIDDVIDEIISLEIDDVIDE | TAD-14 (amino |
| NO: | IISLEIDDVIDEIISLEIDD | acid sequence) |
| 16 | VIDEIISLGSDNLDFDLDLM | |
| | PDNLDFDLDLMPDNLDFDLD | |
| | LMPDNLDFDLDLMPGSLDED | |
| | WDSALFAELGYFTDTDELQL | |
| | EAANETYENNFDNLDFDLDL | |
| | MP | |
| SEQ ID | DNLDFDLDLMPDNLDFDLDL | TAD-15 (amino |
| NO: | MPDNLDFDLDLMPDNLDFDL | acid sequence) |
| 17 | DLMPSGVTPPPPATTNSTNP | |
| | SPQGSHSAIGLSGLNPSTGP | |
| | GLWWNPAPYQPGSLDEDWDS | |
| | ALFAELGYFTDTDELQLEAA | |
| | NETYENNFDNLDFDLDLMP | |
| SEQ ID | LDEDWDSALFAELGYFTDTD | TAD-16 (amino |
| NO: | ELQLEAANETYENNFDNLDF | acid sequence) |
| 18 | DLDLMPSGVTPPPPATTNST | |
| | NPSPQGSHSAIGLSGLNPST | |
| | GPGLWWNPAPYQPDNLDFDL | |
| | DLMPDNLDFDLDLMPDNLDF | |
| | DLDLMPDNLDFDLDLMP | |
| SEQ ID | DDFDLDL | VP7 (amino acid |
| NO: | | sequence) |
| 19 | | |
| SEQ ID | EIDDVIDEIISL | TEF3 (amino |
| NO: | | acid sequence) |
| 20 | | |
| SEQ ID | DNLDFDLDLMP | ATF6-11 (amino |
| NO: | | acid sequence) |
| 21 | | |
| SEQ ID | LDEDWDSALFAELGYFTDTD | ATF6-Acidic |
| NO: | ELQLEAANETYENNFDNLDF | (amino acid |
| 22 | DLDLMP | sequence) |
| SEQ ID | QYVVAAAPNLQNQQVLTGLP | SP1 Q-rich |
| NO: | GVMPNIQYQVIPQFQTVDGQ | (amino acid |
| 23 | QLQFAATGAQVQQDGSGQIQ | sequence) |
| | IIPGANQQIITNRGSGGNII | |
| | AAMPNLLQQAVPLQGLANNV | |
| | LSGQTQYV | |
| SEQ ID | TGSQLAGDIQQLLQLQQLVL | Oct2 P-rich |
| NO: | VPGHHLQPPAQFLLPQAQQS | (amino acid |
| 24 | QPGLLPTPNLFQLPQQTQGA | sequence) |
| | LLTSQP | |
| SEQ ID | DFQPPYFPPPYQPIYPQSQD | TFAP2 P-rich |
| NO: | PYSHVNDPYSLNPLHAQPQP | (amino acid |
| 25 | QHPGWPGQRQSQESGLL | sequence) |
| SEQ ID | DALDDFDLDMLGSDALDDFD | VP64 (amino |
| NO: | LDMLGSDALDDFDLDMLGSD | acid sequence) |
| 26 | ALDDFDLDML | |
| SEQ ID | QYLPDTDDRHRIEEKRKRTY | P65 (amino acid |
| NO: | ETFKSIMKKSPFSGPTDPRP | sequence) |
| 27 | PPRRIAVPSRSSASVPKPAP | |
| | QPYPFTSSLSTINYDEFPTM | |
| | VFPSGQISQASALAPAPPQV | |
| | LPQAPAPAPAPAMVSALAQA | |
| | PAPVPVLAPGPPQAVAPPAP | |
| | KPTQAGEGTLSEALLQLQFD | |
| | DEDLGALLGNSTDPAVFTDL | |
| | ASVDNSEFQQLLNQGIPVAP | |
| | HTTEPMLMEYPEAITRLVTG | |
| | AQRPPDPAPAPLGAPGLPNG | |
| | LLSGDEDFSSIADMDFSALL | |
| SEQ ID | RDSREGMFLPKPEAGSAISD | Rta (amino acid |
| NO: | VFEGREVCQPKRIRPFHPPG | sequence) |
| 28 | SPWANRPLPASLAPTPTGPV | |
| | HEPVGSLTPAPVPQPLDPAP | |
| | AVTPEASHLLEDPDEETSQA | |
| | VKALREMADTVIPQKEEAAI | |
| | CGQMDLSHPPPRGHLDELTT | |
| | TLESMTEDLNLDSPLTPELN | |
| | EILDTFLNDECLLHAMHIST | |
| | GLSIFDTSLF | |
| SEQ ID | TGSQLAGDIQQLLQLQQLVL | Oct2 Q-rich |
| NO: | VPGHHLQPPAQFLLPQAQQS | (amino acid |
| 29 | QPGLLPTPNLFQLPQQTQGA | sequence) |
| | LLTSQP | |
| SEQ ID | GATGACTTCGATCTCGATCT | TAD-1 (nucleic |
| NO: | TGACGATTTCGACCTGGACT | acid sequence) |
| 30 | TGGACGATTTTGATCTGGAT | |
| | CTTGACGACTTTGACTTGGA | |
| | CCTTTCCTCCGGTTCTCCTA | |
| | AAAAGAAGAGGAAGGTCGGA | |
| | TCAACGGGCAGCCAGCTGGC | |
| | TGGAGATATACAGCAGCTCC | |
| | TCCAGCTGCAGCAGCTGGTG | |
| | CTTGTGCCAGGACACCATCT | |
| | GCAGCCACCTGCTCAGTTCC | |
| | TGCTACCGCAGGCCCAGCAG | |
| | AGCCAGCCAGGCCTGCTACC | |
| | GACACCAAATCTATTCCAGC | |
| | TACCTCAGCAAACCCAGGGA | |
| | GCTCTTCTGACCTCCCAGCC | |
| | CGGCGGGTCAGGAGACTTCC | |
| | AGCCCCCATACTTCCCTCCA | |
| | CCCTACCAGCCTATCTACCC | |
| | CCAGTCGCAAGATCCTTACT | |
| | CTCACGTCAACGATCCTTAC | |
| | TCTCTGAATCCACTGCATGC | |
| | TCAGCCTCAGCCTCAGCATC | |
| | CAGGATGGCCTGGCCAGAGG | |
| | CAGAGCCAGGAGTCTGGGCT | |
| | CCTGGGGAGCGGACTGGATG | |
| | AAGATTGGGATTCTGCTCTC | |
| | TTTGCTGAACTCGGTTATTT | |
| | CACAGACACTGATGAGCTGC | |
| | AATTGGAAGCAGCAAATGAG | |
| | ACGTATGAAAACAATTTTGA | |
| | TAATCTTGATTTTGATTTGG | |
| | ATTTGATGCCT | |
| SEQ ID | GACGCATTGGACGATTTTGA | TAD-2 Minus |
| NO: | TCTGGATATGCTGGGAAGTG | (nucleic acid |
| 31 | ACGCCCTCGATGATTTTGAC | sequence) |
| | CTTGACATGCTTGGTTCGGA | |
| | TGCCCTTGATGACTTTGACC | |
| | TCGACATGCTCGGCAGTGAC | |
| | GCCCTTGATGATTTCGACCT | |
| | GGACATGCTGATTAACTCTA | |
| | GAAGTTCCGGATCAACGGGC | |
| | AGCCAGCTGGCTGGAGATAT | |
| | ACAGCAGCTCCTCCAGCTGC | |
| | AGCAGCTGGTGCTTGTGCCA | |
| | GGACACCATCTGCAGCCACC | |
| | TGCTCAGTTCCTGCTACCGC | |
| | AGGCCCAGCAGAGCCAGCCA | |
| | GGCCTGCTACCGACACCAAA | |
| | TCTATTCCAGCTACCTCAGC | |
| | AAACCCAGGGAGCTCTTCTG | |
| | ACCTCCCAGCCCGGCGGGTC | |
| | AGGAGACTTCCAGCCCCCAT | |
| | ACTTCCCTCCACCCTACCAG | |
| | CCTATCTACCCCCAGTCGCA | |
| | AGATCCTTACTCTCACGTCA | |
| | ACGATCCTTACTCTCTGAAT | |
| | CCACTGCATGCTCAGCCTCA | |
| | GCCTCAGCATCCAGGATGGC | |
| | CTGGCCAGAGGCAGAGCCAG | |
| | GAGTCTGGGCTCCTGGGGAG | |
| | CGGACTGGATGAAGATTGGG | |
| | ATTCTGCTCTCTTTGCTGAA | |
| | CTCGGTTATTTCACAGACAC | |
| | TGATGAGCTGCAATTGGAAG | |
| | CAGCAAATGAGACGTATGAA | |
| | AACAATTTTGATAATCTTGA | |
| | TTTTGATTTGGATTTGATGC | |
| | CT | |
| SEQ ID | GACGCATTGGACGATTTTGA | TAD-2 Plus |
| NO: | TCTGGATATGCTGGGAAGTG | (nucleic acid |
| 32 | ACGCCCTCGATGATTTTGAC | sequence) |
| | CTTGACATGCTTGGTTCGGA | |
| | TGCCCTTGATGACTTTGACC | |
| | TCGACATGCTCGGCAGTGAC | |
| | GCCCTTGATGATTTCGACCT | |
| | GGACATGCTGATTAACTCCT | |
| | CCGGTTCTCCTAAAAAGAAG | |
| | AGGAAGGTCGGATCAACGGG | |
| | CAGCCAGCTGGCTGGAGATA | |
| | TACAGCAGCTCCTCCAGCTG | |
| | CAGCAGCTGGTGCTTGTGCC | |
| | AGGACACCATCTGCAGCCAC | |
| | CTGCTCAGTTCCTGCTACCG | |
| | CAGGCCCAGCAGAGCCAGCC | |
| | AGGCCTGCTACCGACACCAA | |
| | ATCTATTCCAGCTACCTCAG | |
| | CAAACCCAGGGAGCTCTTCT | |
| | GACCTCCCAGCCCGGCGGGT | |
| | CAGGAGACTTCCAGCCCCCA | |
| | TACTTCCCTCCACCCTACCA | |
| | GCCTATCTACCCCCAGTCGC | |
| | AAGATCCTTACTCTCACGTC | |
| | AACGATCCTTACTCTCTGAA | |
| | TCCACTGCATGCTCAGCCTC | |
| | AGCCTCAGCATCCAGGATGG | |
| | CCTGGCCAGAGGCAGAGCCA | |
| | GGAGTCTGGGCTCCTGGGGA | |
| | GCGGACTGGATGAAGATTGG | |
| | GATTCTGCTCTCTTTGCTGA | |
| | ACTCGGTTATTTCACAGACA | |
| | CTGATGAGCTGCAATTGGAA | |
| | GCAGCAAATGAGACGTATGA | |
| | AAACAATTTTGATAATCTTG | |
| | ATTTTGATTTGGATTTGATG | |
| | CCT | |
| SEQ ID | GAAATTGACGACGTGATAGA | TAD-3 (nucleic |
| NO: | TGAGATCATAAGCCTGGAAA | acid sequence) |
| 33 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCGGACAATACGTAG | |
| | TTGCTGCGGCGCCTAACCTG | |
| | CAGAACCAGCAGGTACTCAC | |
| | CGGGCTGCCCGGGGTCATGC | |
| | CGAATATACAGTACCAGGTA | |
| | ATCCCTCAATTCCAAACGGT | |
| | GGACGGTCAACAGCTGCAGT | |
| | TCGCCGCAACGGGAGCACAG | |
| | GTGCAGCAAGATGGTAGCGG | |
| | GCAAATCCAAATTATACCGG | |
| | GCGCGAATCAACAGATTATC | |
| | ACAAATCGCGGCAGCGGAGG | |
| | CAACATTATCGCAGCCATGC | |
| | CTAATCTGTTGCAACAGGCT | |
| | GTGCCCCTCCAGGGACTCGC | |
| | CAACAACGTGCTTAGTGGGC | |
| | AAACGCAGTACGTCACGCCA | |
| | CCGCCCCCTGCGACGACAAA | |
| | TTCTACAAATCCCAGTCCGC | |
| | AGGGTAGCCACTCCGCAATA | |
| | GGCCTGTCCGGACTGAATCC | |
| | TTCAACTGGACCGGGACTTT | |
| | GGTGGAATCCGGCTCCTTAC | |
| | CAACCGGATAATCTGGACTT | |
| | CGACTTGGATCTTATGCCAG | |
| | ACAACCTTGATTTTGATCTG | |
| | GATCTTATGCCGGATAATCT | |
| | TGACTTTGATCTCGATCTTA | |
| | TGCCGGACAACTTGGACTTC | |
| | GATCTGGATCTGATGCCA | |
| SEQ ID | GATGACTTCGATCTCGATCT | TAD-4 (nucleic |
| NO: | TGACGATTTCGACCTGGACT | acid sequence) |
| 34 | TGGACGATTTTGATCTGGAT | |
| | CTTGACGACTTTGACTTGGA | |
| | CCTTTCCTCCGGTTCTGACT | |
| | TCCAGCCCCCATACTTCCCT | |
| | CCACCCTACCAGCCTATCTA | |
| | CCCCCAGTCGCAAGATCCTT | |
| | ACTCTCACGTCAACGATCCT | |
| | TACTCTCTGAATCCACTGCA | |
| | TGCTCAGCCTCAGCCTCAGC | |
| | ATCCAGGATGGCCTGGCCAG | |
| | AGGCAGAGCCAGGAGTCTGG | |
| | GCTCCTGGGCGGGTCAGGAA | |
| | CGGGCAGCCAGCTGGCTGGA | |
| | GATATACAGCAGCTCCTCCA | |
| | GCTGCAGCAGCTGGTGCTTG | |
| | TGCCAGGACACCATCTGCAG | |
| | CCACCTGCTCAGTTCCTGCT | |
| | ACCGCAGGCCCAGCAGAGCC | |
| | AGCCAGGCCTGCTACCGACA | |
| | CCAAATCTATTCCAGCTACC | |
| | TCAGCAAACCCAGGGAGCTC | |
| | TTCTGACCTCCCAGCCCGGG | |
| | AGCGGACTGGATGAAGATTG | |
| | GGATTCTGCTCTCTTTGCTG | |
| | AACTCGGTTATTTCACAGAC | |
| | ACTGATGAGCTGCAATTGGA | |
| | AGCAGCAAATGAGACGTATG | |
| | AAAACAATTTTGATAATCTT | |
| | GATTTTGATTTGGATTTGAT | |
| | GCCT | |
| SEQ ID | GATGACTTCGATCTCGATCT | TAD-5 (nucleic |
| NO: | TGACGATTTCGACCTGGACT | acid sequence) |
| 35 | TGGACGATTTTGATCTGGAT | |
| | CTTGACGACTTTGACTTGGA | |
| | CCTTTCCTCCGGTTCTACGG | |
| | GCAGCCAGCTGGCTGGAGAT | |
| | ATACAGCAGCTCCTCCAGCT | |
| | GCAGCAGCTGGTGCTTGTGC | |
| | CAGGACACCATCTGCAGCCA | |
| | CCTGCTCAGTTCCTGCTACC | |
| | GCAGGCCCAGCAGAGCCAGC | |
| | CAGGCCTGCTACCGACACCA | |
| | AATCTATTCCAGCTACCTCA | |
| | GCAAACCCAGGGAGCTCTTC | |
| | TGACCTCCCAGCCCGGCGGG | |
| | TCAGGACTGGATGAAGATTG | |
| | GGATTCTGCTCTCTTTGCTG | |
| | AACTCGGTTATTTCACAGAC | |
| | ACTGATGAGCTGCAATTGGA | |
| | AGCAGCAAATGAGACGTATG | |
| | AAAACAATTTTGATAATCTT | |
| | GATTTTGATTTGGATTTGAT | |
| | GCCTGGGAGCGGAGACTTCC | |
| | AGCCCCCATACTTCCCTCCA | |
| | CCCTACCAGCCTATCTACCC | |
| | CCAGTCGCAAGATCCTTACT | |
| | CTCACGTCAACGATCCTTAC | |
| | TCTCTGAATCCACTGCATGC | |
| | TCAGCCTCAGCCTCAGCATC | |
| | CAGGATGGCCTGGCCAGAGG | |
| | CAGAGCCAGGAGTCTGGGCT | |
| | CCTG | |
| SEQ ID | GATGACTTCGATCTCGATCT | TAD-6 (nucleic |
| NO: | TGACGATTTCGACCTGGACT | acid sequence) |
| 36 | TGGACGATTTTGATCTGGAT | |
| | CTTGACGACTTTGACTTGGA | |
| | CCTTTCCTCCGGTTCTCCTA | |
| | AAAAGAAGAGGAAGGTCGGA | |
| | TCAACGGGCAGCCAGCTGGC | |
| | TGGAGATATACAGCAGCTCC | |
| | TCCAGCTGCAGCAGCTGGTG | |
| | CTTGTGCCAGGACACCATCT | |
| | GCAGCCACCTGCTCAGTTCC | |
| | TGCTACCGCAGGCCCAGCAG | |
| | AGCCAGCCAGGCCTGCTACC | |
| | GACACCAAATCTATTCCAGC | |
| | TACCTCAGCAAACCCAGGGA | |
| | GCTCTTCTGACCTCCCAGCC | |
| | CGGCGGGTCAGGAGTCACGC | |
| | CACCGCCCCCTGCGACGACA | |
| | AATTCTACAAATCCCAGTCC | |
| | GCAGGGTAGCCACTCCGCAA | |
| | TAGGCCTGTCCGGACTGAAT | |
| | CCTTCAACTGGACCGGGACT | |
| | TTGGTGGAATCCGGCTCCTT | |
| | ACCAACCGGATAATCTGGAC | |
| | TTCGACTTGGATCTTATGCC | |
| | AGACAACCTTGATTTTGATC | |
| | TGGATCTTATGCCGGATAAT | |
| | CTTGACTTTGATCTCGATCT | |
| | TATGCCGGACAACTTGGACT | |
| | TCGATCTGGATCTGATGCCA | |
| SEQ ID | GAAATTGACGACGTGATAGA | TAD-7 (nucleic |
| NO: | TGAGATCATAAGCCTGGAAA | acid sequence) |
| 37 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAACGGGCAGCC | |
| | AGCTGGCTGGAGATATACAG | |
| | CAGCTCCTCCAGCTGCAGCA | |
| | GCTGGTGCTTGTGCCAGGAC | |
| | ACCATCTGCAGCCACCTGCT | |
| | CAGTTCCTGCTACCGCAGGC | |
| | CCAGCAGAGCCAGCCAGGCC | |
| | TGCTACCGACACCAAATCTA | |
| | TTCCAGCTACCTCAGCAAAC | |
| | CCAGGGAGCTCTTCTGACCT | |
| | CCCAGCCCGGCGGGTCAGGA | |
| | GTCACGCCACCGCCCCCTGC | |
| | GACGACAAATTCTACAAATC | |
| | CCAGTCCGCAGGGTAGCCAC | |
| | TCCGCAATAGGCCTGTCCGG | |
| | ACTGAATCCTTCAACTGGAC | |
| | CGGGACTTTGGTGGAATCCG | |
| | GCTCCTTACCAACCGGATAA | |
| | TCTGGACTTCGACTTGGATC | |
| | TTATGCCAGACAACCTTGAT | |
| | TTTGATCTGGATCTTATGCC | |
| | GGATAATCTTGACTTTGATC | |
| | TCGATCTTATGCCGGACAAC | |
| | TTGGACTTCGATCTGGATCT | |
| | GATGCCA | |
| SEQ ID | GAAATTGACGACGTGATAGA | TAD-8a (nucleic |
| NO: | TGAGATCATAAGCCTGGAAA | acid sequence) |
| 38 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAACGGGCAGCC | |
| | AGCTGGCTGGAGATATACAG | |
| | CAGCTCCTCCAGCTGCAGCA | |
| | GCTGGTGCTTGTGCCAGGAC | |
| | ACCATCTGCAGCCACCTGCT | |
| | CAGTTCCTGCTACCGCAGGC | |
| | CCAGCAGAGCCAGCCAGGCC | |
| | TGCTACCGACACCAAATCTA | |
| | TTCCAGCTACCTCAGCAAAC | |
| | CCAGGGAGCTCTTCTGACCT | |
| | CCCAGCCCGGCGGGGATGAC | |
| | TTCGATCTCGATCTTGACGA | |
| | TTTCGACCTGGACTTGGACG | |
| | ATTTTGATCTGGATCTTGAC | |
| | GACTTTGACTTGGACCTTTC | |
| | AGGAGTCACGCCACCGCCCC | |
| | CTGCGACGACAAATTCTACA | |
| | AATCCCAGTCCGCAGGGTAG | |
| | CCACTCCGCAATAGGCCTGT | |
| | CCGGACTGAATCCTTCAACT | |
| | GGACCGGGACTTTGGT | |
| SEQ ID | GGAATCCGGCTCCTTACCAA | TAD-8b (nucleic |
| NO: | CCGGATAATCTGGACTTCGA | acid sequence) |
| 39 | CTTGGATCTTATGCCAGACA | |
| | ACCTTGATTTTGATCTGGAT | |
| | CTTATGCCGGATAATCTTGA | |
| | CTTTGATCTCGATCTTATGC | |
| | CGGACAACTTGGACTTCGAT | |
| | CTGGATCTGATGCCAGAAAT | |
| | TGACGACGTGATAGATGAGA | |
| | TCATAAGCCTGGAAATAGAC | |
| | GACGTTATAGACGAGATAAT | |
| | ATCACTTGAGATAGATGATG | |
| | TCATCGACGAAATCATCAGT | |
| | CTGGAAATCGACGACGTAAT | |
| | CGATGAAATTATCTCATTGG | |
| | GCTCAACGGGCAGCCAGCTG | |
| | GCTGGAGATATACAGCAGCT | |
| | CCTCCAGCTGCAGCAGCTGG | |
| | TGCTTGTGCCAGGACACCAT | |
| | CTGCAGCCACCTGCTCAGTT | |
| | CCTGCTACCGCAGGCCCAGC | |
| | AGAGCCAGCCAGGCCTGCTA | |
| | CCGACACCAAATCTATTCCA | |
| | GCTACCTCAGCAAACCCAGG | |
| | GAGCTCTTCTGACCTCCCAG | |
| | CCCGGCGGGCTGGATGAAGA | |
| | TTGGGATTCTGCTCTCTTTG | |
| | CTGAACTCGGTTATTTCACA | |
| | GACACTGATGAGCTGCAATT | |
| | GGAAGCAGCAAATGAGACGT | |
| | ATGAAAACAATTTTGATAAT | |
| | CTTGATTTTGATTTGGATTT | |
| | GATGCCTTCAGGAGTCACGC | |
| | CACCGCCCCCTGCGACGACA | |
| | AATTCTACAAATCCCAGTCC | |
| | GCAGGGTAGCCACTCCGCAA | |
| | TAGGCCTGTCCGGACTGAAT | |
| | CCTTCAACTGGACCGGGACT | |
| | TTGGTGGAATCCGGCTCCTT | |
| | ACCAACCGGATAATCTGGAC | |
| | TTCGACTTGGATCTTATGCC | |
| | AGACAACCTTGATTTTGATC | |
| | TGGATCTTATGCCGGATAAT | |
| | CTTGACTTTGATCTCGATCT | |
| | TATGCCGGACAACTTGGACT | |
| | TCGATCTGGATCTGATGCCA | |
| SEQ ID | GAAATTGACGACGTGATAGA | TAD-9 (nucleic |
| NO: | TGAGATCATAAGCCTGGAAA | acid sequence) |
| 40 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCACTGGATGAAG | |
| | ATTGGGATTCTGCTCTCTTT | |
| | GCTGAACTCGGTTATTTCAC | |
| | AGACACTGATGAGCTGCAAT | |
| | TGGAAGCAGCAAATGAGACG | |
| | TATGAAAACAATTTTGATAA | |
| | TCTTGATTTTGATTTGGATT | |
| | TGATGCCTTCAGGAGTCACG | |
| | CCACCGCCCCCTGCGACGAC | |
| | AAATTCTACAAATCCCAGTC | |
| | CGCAGGGTAGCCACTCCGCA | |
| | ATAGGCCTGTCCGGACTGAA | |
| | TCCTTCAACTGGACCGGGAC | |
| | TTTGGTGGAATCCGGCTCCT | |
| | TACCAACCGGATAATCTGGA | |
| | CTTCGACTTGGATCTTATGC | |
| | CAGACAACCTTGATTTTGAT | |
| | CTGGATCTTATGCCGGATAA | |
| | TCTTGACTTTGATCTCGATC | |
| | TTATGCCGGACAACTTGGAC | |
| | TTCGATCTGGATCTGATGCC | |
| | A | |
| SEQ ID | GAAATTGACGACGTGATAGA | TAD-10 (nucleic |
| NO: | TGAGATCATAAGCCTGGAAA | acid sequence) |
| 41 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCACTGGATGAAG | |
| | ATTGGGATTCTGCTCTCTTT | |
| | GCTGAACTCGGTTATTTCAC | |
| | AGACACTGATGAGCTGCAAT | |
| | TGGAAGCAGCAAATGAGACG | |
| | TATGAAAACAATTTTGATAA | |
| | TCTTGATTTTGATTTGGATT | |
| | TGATGCCTTCAGGAACGGGC | |
| | AGCCAGCTGGCTGGAGATAT | |
| | ACAGCAGCTCCTCCAGCTGC | |
| | AGCAGCTGGTGCTTGTGCCA | |
| | GGACACCATCTGCAGCCACC | |
| | TGCTCAGTTCCTGCTACCGC | |
| | AGGCCCAGCAGAGCCAGCCA | |
| | GGCCTGCTACCGACACCAAA | |
| | TCTATTCCAGCTACCTCAGC | |
| | AAACCCAGGGAGCTCTTCTG | |
| | ACCTCCCAGCCCGGCGGGGA | |
| | TAATCTGGACTTCGACTTGG | |
| | ATCTTATGCCAGACAACCTT | |
| | GATTTTGATCTGGATCTTAT | |
| | GCCGGATAATCTTGACTTTG | |
| | ATCTCGATCTTATGCCGGAC | |
| | AACTTGGACTTCGATCTGGA | |
| | TCTGATGCCA | |
| SEQ ID | GAAATTGACGACGTGATAGA | TAD-11 (nucleic |
| NO: | TGAGATCATAAGCCTGGAAA | acid sequence) |
| 42 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAGATAATCTGG | |
| | ACTTCGACTTGGATCTTATG | |
| | CCAGACAACCTTGATTTTGA | |
| | TCTGGATCTTATGCCGGATA | |
| | ATCTTGACTTTGATCTCGAT | |
| | CTTATGCCGGACAACTTGGA | |
| | CTTCGATCTGGATCTGATGC | |
| | CATCAGGAGTCACGCCACCG | |
| | CCCCCTGCGACGACAAATTC | |
| | TACAAATCCCAGTCCGCAGG | |
| | GTAGCCACTCCGCAATAGGC | |
| | CTGTCCGGACTGAATCCTTC | |
| | AACTGGACCGGGACTTTGGT | |
| | GGAATCCGGCTCCTTACCAA | |
| | CCGGGCTCACTGGATGAAGA | |
| | TTGGGATTCTGCTCTCTTTG | |
| | CTGAACTCGGTTATTTCACA | |
| | GACACTGATGAGCTGCAATT | |
| | GGAAGCAGCAAATGAGACGT | |
| | ATGAAAACAATTTTGATAAT | |
| | CTTGATTTTGATTTGGATTT | |
| | GATGCCT | |
| SEQ ID | GAAATTGACGACGTGATAGA | TAD-12 (nucleic |
| NO: | TGAGATCATAAGCCTGGAAA | acid sequence) |
| 43 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAGATAATCTGG | |
| | ACTTCGACTTGGATCTTATG | |
| | CCAGACAACCTTGATTTTGA | |
| | TCTGGATCTTATGCCGGATA | |
| | ATCTTGACTTTGATCTCGAT | |
| | CTTATGCCGGACAACTTGGA | |
| | CTTCGATCTGGATCTGATGC | |
| | CATCAGGAGTCACGCCACCG | |
| | CCCCCTGCGACGACAAATTC | |
| | TACAAATCCCAGTCCGCAGG | |
| | GTAGCCACTCCGCAATAGGC | |
| | CTGTCCGGACTGAATCCTTC | |
| | AACTGGACCGGGACTTTGGT | |
| | GGAATCCGGCTCCTTACCAA | |
| | CCG | |
| SEQ ID | GAAATTGACGACGTGATAGA | TAD-13 (nucleic |
| NO: | TGAGATCATAAGCCTGGAAA | acid sequence) |
| 44 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAGTCACGCCAC | |
| | CGCCCCCTGCGACGACAAAT | |
| | TCTACAAATCCCAGTCCGCA | |
| | GGGTAGCCACTCCGCAATAG | |
| | GCCTGTCCGGACTGAATCCT | |
| | TCAACTGGACCGGGACTTTG | |
| | GTGGAATCCGGCTCCTTACC | |
| | AACCGGATAATCTGGACTTC | |
| | GACTTGGATCTTATGCCAGA | |
| | CAACCTTGATTTTGATCTGG | |
| | ATCTTATGCCGGATAATCTT | |
| | GACTTTGATCTCGATCTTAT | |
| | GCCGGACAACTTGGACTTCG | |
| | ATCTGGATCTGATGCCA | |
| SEQ ID | GAAATTGACGACGTGATAGA | TAD-14 (nucleic |
| NO: | TGAGATCATAAGCCTGGAAA | acid sequence) |
| 45 | TAGACGACGTTATAGACGAG | |
| | ATAATATCACTTGAGATAGA | |
| | TGATGTCATCGACGAAATCA | |
| | TCAGTCTGGAAATCGACGAC | |
| | GTAATCGATGAAATTATCTC | |
| | ATTGGGCTCAGATAATCTGG | |
| | ACTTCGACTTGGATCTTATG | |
| | CCAGACAACCTTGATTTTGA | |
| | TCTGGATCTTATGCCGGATA | |
| | ATCTTGACTTTGATCTCGAT | |
| | CTTATGCCGGACAACTTGGA | |
| | CTTCGATCTGGATCTGATGC | |
| | CAGGCTCACTGGATGAAGAT | |
| | TGGGATTCTGCTCTCTTTGC | |
| | TGAACTCGGTTATTTCACAG | |
| | ACACTGATGAGCTGCAATTG | |
| | GAAGCAGCAAATGAGACGTA | |
| | TGAAAACAATTTTGATAATC | |
| | TTGATTTTGATTTGGATTTG | |
| | ATGCCT | |
| SEQ ID | GATAATCTGGACTTCGACTT | TAD-15 (nucleic |
| NO: | GGATCTTATGCCAGACAACC | acid sequence) |
| 46 | TTGATTTTGATCTGGATCTT | |
| | ATGCCGGATAATCTTGACTT | |
| | TGATCTCGATCTTATGCCGG | |
| | ACAACTTGGACTTCGATCTG | |
| | GATCTGATGCCATCAGGAGT | |
| | CACGCCACCGCCCCCTGCGA | |
| | CGACAAATTCTACAAATCCC | |
| | AGTCCGCAGGGTAGCCACTC | |
| | CGCAATAGGCCTGTCCGGAC | |
| | TGAATCCTTCAACTGGACCG | |
| | GGACTTTGGTGGAATCCGGC | |
| | TCCTTACCAACCGGGCTCAC | |
| | TGGATGAAGATTGGGATTCT | |
| | GCTCTCTTTGCTGAACTCGG | |
| | TTATTTCACAGACACTGATG | |
| | AGCTGCAATTGGAAGCAGCA | |
| | AATGAGACGTATGAAAACAA | |
| | TTTTGATAATCTTGATTTTG | |
| | ATTTGGATTTGATGCCT | |
| SEQ ID | CTGGATGAAGATTGGGATTC | TAD-16 (nucleic |
| NO: | TGCTCTCTTTGCTGAACTCG | acid sequence) |
| 47 | GTTATTTCACAGACACTGAT | |
| | GAGCTGCAATTGGAAGCAGC | |
| | AAATGAGACGTATGAAAACA | |
| | ATTTTGATAATCTTGATTTT | |
| | GATTTGGATTTGATGCCTTC | |
| | AGGAGTCACGCCACCGCCCC | |
| | CTGCGACGACAAATTCTACA | |
| | AATCCCAGTCCGCAGGGTAG | |
| | CCACTCCGCAATAGGCCTGT | |
| | CCGGACTGAATCCTTCAACT | |
| | GGACCGGGACTTTGGTGGAA | |
| | TCCGGCTCCTTACCAACCGG | |
| | ATAATCTGGACTTCGACTTG | |
| | GATCTTATGCCAGACAACCT | |
| | TGATTTTGATCTGGATCTTA | |
| | TGCCGGATAATCTTGACTTT | |
| | GATCTCGATCTTATGCCGGA | |
| | CAACTTGGACTTCGATCTGG | |
| | ATCTGATGCCA | |
| SEQ ID | GATGACTTCGATCTCGATCT | VP7 (nucleic |
| NO: | T | acid sequence) |
| 48 | | |
| SEQ ID | GAAATTGACGACGTGATAGA | TEF3 (nucleic |
| NO: | TGAGATCATAAGCCTG | acid sequence) |
| 49 | | |
| SEQ ID | GATAATCTGGACTTCGACTT | ATF6-11 (nucleic |
| NO: | GGATCTTATGCCA | acid sequence) |
| 50 | | |
| SEQ ID | CTGGATGAAGATTGGGATTC | ATF6-Acidic |
| NO: | TGCTCTCTTTGCTGAACTCG | (nucleic acid |
| 51 | GTTATTTCACAGACACTGAT | sequence) |
| | GAGCTGCAATTGGAAGCAGC | |
| | AAATGAGACGTATGAAAACA | |
| | ATTTTGATAATCTTGATTTT | |
| | GATTTGGATTTGATGCCT | |
| SEQ ID | QCAATACGTAGTTGCTGCGG | SP1 Q-rich |
| NO: | CGCCTAACCTGCAGAACCAG | (nucleic acid |
| 52 | CAGGTACTCACCGGGCTGCC | sequence) |
| | CGGGGTCATGCCGAATATAC | |
| | AGTACCAGGTAATCCCTCAA | |
| | TTCCAAACGGTGGACGGTCA | |
| | ACAGCTGCAGTTCGCCGCAA | |
| | CGGGAGCACAGGTGCAGCAA | |
| | GATGGTAGCGGGCAAATCCA | |
| | AATTATACCGGGCGCGAATC | |
| | AACAGATTATCACAAATCGC | |
| | GGCAGCGGAGGCAACATTAT | |
| | CGCAGCCATGCCTAATCTGT | |
| | TGCAACAGGCTGTGCCCCTC | |
| | CAGGGACTCGCCAACAACGT | |
| | GCTTAGTGGGCAAACGCAGT | |
| | ACGTC | |
| SEQ ID | ACGGGCAGCCAGCTGGCTGG | Oct2 Q-rich |
| NO: | AGATATACAGCAGCTCCTCC | (nucleic acid |
| 53 | AGCTGCAGCAGCTGGTGCTT | sequence) |
| | GTGCCAGGACACCATCTGCA | |
| | GCCACCTGCTCAGTTCCTGC | |
| | TACCGCAGGCCCAGCAGAGC | |
| | CAGCCAGGCCTGCTACCGAC | |
| | ACCAAATCTATTCCAGCTAC | |
| | CTCAGCAAACCCAGGGAGCT | |
| | CTTCTGACCTCCCAGCCC | |
| SEQ ID | GACTTCCAGCCCCCATACTT | TFAP2 P-rich |
| NO: | CCCTCCACCCTACCAGCCTA | (nucleic acid |
| 54 | TCTACCCCCAGTCGCAAGAT | sequence) |
| | CCTTACTCTCACGTCAACGA | |
| | TCCTTACTCTCTGAATCCAC | |
| | TGCATGCTCAGCCTCAGCCT | |
| | CAGCATCCAGGATGGCCTGG | |
| | CCAGAGGCAGAGCCAGGAGT | |
| | CTGGGCTCCTG | |
| SEQ ID | GACGCATTGGACGATTTTGA | VP64 (nucleic |
| NO: | TCTGGATATGCTGGGAAGTG | acid sequence) |
| 55 | ACGCCCTCGATGATTTTGAC | |
| | CTTGACATGCTTGGTTCGGA | |
| | TGCCCTTGATGACTTTGACC | |
| | TCGACATGCTCGGCAGTGAC | |
| | GCCCTTGATGATTTCGACCT | |
| | GGACATGCTG | |
| SEQ ID | CAGTACCTGCCCGACACCGA | P65 (nucleic acid |
| NO: | CGACCGGCACCGGATCGAGG | sequence) |
| 56 | AAAAGCGGAAGCGGACCTAC | |
| | GAGACATTCAAGAGCATCAT | |
| | GAAGAAGTCCCCCTTCAGCG | |
| | GCCCCACCGACCCTAGACCT | |
| | CCACCTAGAAGAATCGCCGT | |
| | GCCCAGCAGATCCAGCGCCA | |
| | GCGTGCCAAAACCTGCCCCC | |
| | CAGCCTTACCCCTTCACCAG | |
| | CAGCCTGAGCACCATCAACT | |
| | ACGACGAGTTCCCTACCATG | |
| | GTGTTCCCCAGCGGCCAGAT | |
| | CTCTCAGGCCTCTGCTCTGG | |
| | CTCCAGCCCCTCCTCAGGTG | |
| | CTGCCTCAGGCTCCTGCTCC | |
| | TGCACCAGCTCCAGCCATGG | |
| | TGTCTGCACTGGCTCAGGCA | |
| | CCAGCACCCGTGCCTGTGCT | |
| | GGCTCCTGGACCTCCACAGG | |
| | CTGTGGCTCCACCAGCCCCT | |
| | AAACCTACACAGGCCGGCGA | |
| | GGGCACACTGTCTGAAGCTC | |
| | TGCTGCAGCTGCAGTTCGAC | |
| | GACGAGGATCTGGGAGCCCT | |
| | GCTGGGAAACAGCACCGATC | |
| | CTGCCGTGTTCACCGACCTG | |
| | GCCAGCGTGGACAACAGCGA | |
| | GTTCCAGCAGCTGCTGAACC | |
| | AGGGCATCCCTGTGGCCCCT | |
| | CACACCACCGAGCCCATGCT | |
| | GATGGAATACCCCGAGGCCA | |
| | TCACCCGGCTCGTGACAGGC | |
| | GCTCAGAGGCCTCCTGATCC | |
| | AGCTCCTGCCCCTCTGGGAG | |
| | CACCAGGCCTGCCTAATGGA | |
| | CTGCTGTCTGGCGACGAGGA | |
| | CTTCAGCTCTATCGCCGATA | |
| | TGGATTTCTCAGCCTTGCTG | |
| SEQ ID | CGGGATTCCAGGGAAGGGAT | Rta (nucleic acid |
| NO: | GTTTTTGCCGAAGCCTGAGG | sequence) |
| 57 | CCGGCTCCGCTATTAGTGAC | |
| | GTGTTTGAGGGCCGCGAGGT | |
| | GTGCCAGCCAAAACGAATCC | |
| | GGCCATTTCATCCTCCAGGA | |
| | AGTCCATGGGCCAACCGCCC | |
| | ACTCCCCGCCAGCCTCGCAC | |
| | CAACACCAACCGGTCCAGTA | |
| | CATGAGCCAGTCGGGTCACT | |
| | GACCCCGGCACCAGTCCCTC | |
| | AGCCACTGGATCCAGCGCCC | |
| | GCAGTGACTCCCGAGGCCAG | |
| | TCACCTGTTGGAGGATCCCG | |
| | ATGAAGAGACGAGCCAGGCT | |
| | GTCAAAGCCCTTCGGGAGAT | |
| | GGCCGATACTGTGATTCCCC | |
| | AGAAGGAAGAGGCTGCAATC | |
| | TGTGGCCAAATGGACCTTTC | |
| | CCATCCGCCCCCAAGGGGCC | |
| | ATCTGGATGAGCTGACAACC | |
| | ACACTTGAGTCCATGACCGA | |
| | GGATCTGAACCTGGACTCAC | |
| | CCCTGACCCCGGAATTGAAC | |
| | GAGATTCTGGATACCTTCCT | |
| | GAACGACGAGTGCCTCTTGC | |
| | ATGCCATGCATATCAGCACA | |
| | GGACTGTCCATCTTCGACAC | |
| | ATCTCTGTTT | |
| SEQ ID | ACGGGCAGCCAGCTGGCTGG | Oct2 Q-rich |
| NO: | AGATATACAGCAGCTCCTCC | (nucleic acid |
| 58 | AGCTGCAGCAGCTGGTGCTT | sequence) |
| | GTGCCAGGACACCATCTGCA | |
| | GCCACCTGCTCAGTTCCTGC | |
| | TACCGCAGGCCCAGCAGAGC | |
| | CAGCCAGGCCTGCTACCGAC | |
| | ACCAAATCTATTCCAGCTAC | |
| | CTCAGCAAACCCAGGGAGCT | |
| | CTTCTGACCTCCCAGCCC | |

### DNA Binding Domains (DBDs)

The transcription factor provided herein comprises any suitable DBD that binds to a target site of interest (e.g. a target site that results in upregulation of a target gene when bound by a transcription factor provided herein).

### TALEN

In some embodiments, the DBD may be a TALEN. TALENs are produced artificially by fusing a TAL effector DNA binding domain to a DNA cleavage domain. Transcription activator-like effects (TALEs) can be engineered to bind any desired DNA sequence. These can then be introduced into a cell, wherein they can be used for genome editing. Boch, Nature Biotech. 29:135-6 (2011); and Boch et al. Science 326:1509-12 (2009); Moscou et al. Science 326:3501 (2009).

TALEs are proteins secreted by Xanthomonas bacteria. The DNA binding domain contains a repeated, highly conserved 33-34 amino acid sequence, with the exception of the 12th and 13th amino acids. These two positions are highly variable, showing a strong correlation with specific nucleotide recognition. They can thus be engineered to bind to a desired DNA sequence.

TALEs specific to the sequences described herein can be constructed using any method known in the art, including various schemes using modular components. Zhang et al. Nature Biotech. 29:149-53 (2011); Geibler et al. PLOS ONE 6: e19509 (2011); U.S. Pat. Nos. 8,420,782; 8,470,973, the contents of which are hereby incorporated by reference in their entirety.

### CRISPR Gene Editing System

In some embodiments, the DBD can be a gRNA/Cas complex.

A gRNA molecule may have a number of domains, as described more fully below. In some embodiments, a gRNA molecule comprises a targeting domain and interacts with a Cas molecule, such as Cas9. In some embodiments, a gRNA molecule comprises a crRNA domain (comprising a targeting domain) and a tracr. In embodiments, the crRNA and the tracr are provided on a single contiguous polynucleotide molecule. In other embodiments, the crRNA and the tracr are provided on separate polynucleotide molecules, which are themselves capable of association, e.g., through non-covalent hybridization. The gRNA molecules, used as a component of a CRISPR system, are useful for modifying (e.g., modifying the sequence) DNA at or near a target site. Such modifications include deletions and or insertions that result in, for example, reduced or eliminated expression of a functional product of the gene comprising the target site. Such modifications can also include the upregulation of the expression of functional product of the gene comprising the target site, for example, if the Cas9 molecule lacks nuclease activity but is fused to one or more transcription factors. In some embodiments, a separate gRNA molecule and CRISPR system are used to upregulate expression of the functional product of the gene comprising the target site. These uses, and others, are described more fully below.

In an embodiment, a unimolecular, or sgRNA comprises, preferably from 5′ to 3′: a crRNA (which comprises a targeting domain complementary to a target sequence and a region that forms part of a flagpole (i.e., a crRNA flagpole region)); optionally a loop; and a tracr (which comprises a domain complementary to the crRNA flagpole region, and a domain which additionally binds a nuclease or other effector molecule, e.g., a Cas molecule, e.g., a Cas9 molecule), and may take the following format (from 5′ to 3′):
[targeting domain]-[crRNA flagpole region]-[optional first flagpole extension]-[optional loop]-[optional first tracr extension]-[tracr flagpole region]-[tracr nuclease binding domain]. In embodiments, the tracr nuclease binding domain binds to a Cas protein, e.g., a Cas9 protein.

In an embodiment, a bimolecular, or dgRNA comprises two polynucleotides; the first, preferably from 5′ to 3′: a crRNA (which contains a targeting domain complementary to a target sequence and a region that forms part of a flagpole; and the second, preferably from 5′ to 3′: a tracr (which contains a domain complementary to the crRNA flagpole region, and a domain which additionally binds a nuclease or other effector molecule, e.g., a Cas molecule, e.g., Cas9 molecule), and may take the following format (from 5′ to 3′):
Polynucleotide 1 (crRNA): [targeting domain]-[crRNA flagpole region]-[optional first flagpole extension]-[optional second flagpole extension]
Polynucleotide 2 (tracr): [optional first tracr extension]-[tracr flagpole region]-[tracr nuclease binding domain].

In embodiments, the dgRNA comprises two polynucleotides that are covalently linked by non-nucleotide linkers as described in, e.g., He et al., ChemBioChem 17:1809-1812 (2016). In some embodiments a chemistry reaction is used to link the two polynucleotides, for example using a copper (I)-catalyzed alkyne-azide cycloaddition (CuAAC) reaction (see He et al., ChemBioChem 17:1809-1812 (2016)), or through a strain-promoted azide-alkyne cyloaddition (SPAAC) (see US 2016/0215275 A1), both of which are incorporated herein by reference in their entirety. In another embodiment, the two polynucleotides are covalently linked via a thio-ether linker, which can be generated, for example, by reaction between thiol and maleimide functional groups, or by reaction between other functional groups (see, e.g., US 2016/0215275 A1). In yet other embodiments, the non-nucleotide linker can comprise a carbamate, ether, ester, amide, imine, amidine, aminotrizine, hydrozone, disulfide, thioester, phosphorrothioate, phosphorodithioate, sulfonamide, sulfonate, fulfone, sulfoxide, urea, thiourea, hydrazide, oxime, photolabile linkage, or C—C bond forming group such as a Diels-Alder cyclo-addition pair and/or a ring-closing metathesis pair, and/or a Michael reaction pair (see WO 2016/18745 A1, incorporated herein by reference in its entirety).

In some aspects, the flagpole, e.g., the crRNA flagpole region, comprises, from 5′ to 3′: AGUACUCUG.

In some aspects, the loop comprises, from 5′ to 3′: GAAA.

In some aspects the tracr comprises, from 5′ to 3′:

(SEQ ID NO: 79)
AGUACUCUGGAAACAGAAUCUACUCUAACAAGGCAAAAUGCCGUGUUU
AUCUCGUCAACUUGUUGGCGAGAUUUUU.

In some aspects, the gRNA may also comprise, at the 3′ end, additional U nucleic acids. For example the gRNA may comprise an additional 1, 2, 3, 4, 5, 6, 7, 8, 9, or 10 U nucleic acids at the 3′ end. In an embodiment, the gRNA comprises an additional 4-5 U nucleic acids at the 3′ end. In the case of dgRNA, one or more of the polynucleotides of the dgRNA (e.g., the polynucleotide comprising the targeting domain and the polynucleotide comprising the tracr) may comprise, at the 3′ end, additional U nucleic acids. For example, in the case of dgRNA, one or more of the polynucleotides of the dgRNA (e.g., the polynucleotide comprising the targeting domain and the polynucleotide comprising the tracr) may comprise an additional 1, 2, 3, 4, 5, 6, 7, 8, 9, or 10 U nucleic acids at the 3′ end. In an embodiment, in the case of dgRNA, one or more of the polynucleotides of the dgRNA (e.g., the polynucleotide comprising the targeting domain and the polynucleotide comprising the tracr) comprises an additional 4-5 U nucleic acids at the 3′ end. In an embodiment of a dgRNA, only the polynucleotide comprising the tracr comprises the additional U nucleic acid(s), e.g., 4-5 U nucleic acids. In an embodiment of a dgRNA, only the polynucleotide comprising the targeting domain comprises the additional U nucleic acid(s). In an embodiment of a dgRNA, both the polynucleotide comprising the targeting domain and the polynucleotide comprising the tracr comprise the additional U nucleic acids, e.g., 4-5 U nucleic acids.

In some aspects, the gRNA may also comprise, at the 3′ end, additional A nucleic acids. For example the gRNA may comprise an additional 1, 2, 3, 4, 5, 6, 7, 8, 9, or 10 A nucleic acids at the 3′ end. In an embodiment, the gRNA comprises an additional 4 A nucleic acids at the 3′ end. In the case of dgRNA, one or more of the polynucleotides of the dgRNA (e.g., the polynucleotide comprising the targeting domain and the polynucleotide comprising the tracr) may comprise, at the 3′ end, additional A nucleic acids. For example, the case of dgRNA, one or more of the polynucleotides of the dgRNA (e.g., the polynucleotide comprising the targeting domain and the polynucleotide comprising the tracr) may comprise an additional 1, 2, 3, 4, 5, 6, 7, 8, 9, or 10 A nucleic acids at the 3′ end. In an embodiment, in the case of dgRNA, one or more of the polynucleotides of the dgRNA (e.g., the polynucleotide comprising the targeting domain and the polynucleotide comprising the tracr) comprises an additional 4 A nucleic acids at the 3′ end. In an embodiment of a dgRNA, only the polynucleotide comprising the tracr comprises the additional A nucleic acid(s), e.g., 4 A nucleic acids. In an embodiment of a dgRNA, only the polynucleotide comprising the targeting domain comprises the additional A nucleic acid(s). In an embodiment of a dgRNA, both the polynucleotide comprising the targeting domain and the polynucleotide comprising the tracr comprise the additional U nucleic acids, e.g., 4 A nucleic acids.

In embodiments, one or more of the polynucleotides of the gRNA molecule may comprise a cap at the 5′ end.

In an embodiment, a unimolecular, or sgRNA comprises, preferably from 5′ to 3′: a crRNA (which contains a targeting domain complementary to a target sequence; a crRNA flagpole region; first flagpole extension; a loop; a first tracr extension (which contains a domain complementary to at least a portion of the first flagpole extension); and a tracr (which contains a domain complementary to the crRNA flagpole region, and a domain which additionally binds a Cas9 molecule). In some aspects, the targeting domain comprises a targeting domain sequence described herein, or a targeting domain comprising or consisting of 17, 18, 19, 20 (preferably 20) consecutive nucleotides of a targeting domain sequence, for example the 3′ 17, 18, 19 or 20 (preferably 20) consecutive nucleotides of a targeting domain sequence. In embodiments, the 17, 18, 19, 20 (preferably 20) consecutive nucleotides of a targeting domain sequence are the 3′ 17, 18, 19, 20 (preferably 20) consecutive nucleotides of a targeting domain sequence. In embodiments, the 17, 18, 19, 20 (preferably 20) consecutive nucleotides of a targeting domain sequence are the 5′ 17, 18, 19, 20 (preferably 20) consecutive nucleotides of a targeting domain sequence.

In aspects comprising a first flagpole extension and/or a first tracr extension, the flagpole, loop and tracr sequences may be as described above. In general any first flagpole extension and first tracr extension may be employed, provided that they are complementary. In embodiments, the first flagpole extension and first tracr extension consist of 3, 4, 5, 6, 7, 8, 9, 10 or more complementary nucleotides.

In some aspects, the first flagpole extension comprises, from 5′ to 3′: UGCUG. In some aspects, the first flagpole extension consists of SEQ ID NO: 80.

In some aspects, the first tracr extension comprises, from 5′ to 3′: CAGCA. In some aspects, the first tracr extension consists of SEQ ID NO: 81.

In an embodiment, a dgRNA comprises two nucleic acid molecules. In some aspects, the dgRNA comprises a first nucleic acid which contains, preferably from 5′ to 3′: a targeting domain complementary to a target sequence; a crRNA flagpole region; optionally a first flagpole extension; and, optionally, a second flagpole extension; and a second nucleic acid (which may be referred to herein as a tracr), and comprises at least a domain which binds a Cas molecule, e.g., a Cas9 molecule) comprising preferably from 5′ to 3′: optionally a first tracr extension; and a tracr (which contains a domain complementary to the crRNA flagpole region, and a domain which additionally binds a Cas, e.g., Cas9, molecule). The second nucleic acid may additionally comprise, at the 3′ end (e.g., 3′ to the tracr) additional U nucleic acids. For example the tracr may comprise an additional 1, 2, 3, 4, 5, 6, 7, 8, 9, or 10 U nucleic acids at the 3′ end (e.g., 3′ to the tracr). The second nucleic acid may additionally or alternately comprise, at the 3′ end (e.g., 3′ to the tracr) additional A nucleic acids. For example the tracr may comprise an additional 1, 2, 3, 4, 5, 6, 7, 8, 9, or 10 A nucleic acids at the 3′ end (e.g., 3′ to the tracr).

In aspects involving a dgRNA, the crRNA flagpole region, optional first flagpole extension, optional first tracr extension and tracr sequences may be as described above.

In some aspects, the optional second flagpole extension comprises, from 5′ to 3′: UUUUG. In embodiments, the 3′ 1, 2, 3, 4, or 5 nucleotides, the 5′ 1, 2, 3, 4, or 5 nucleotides, or both the 3′ and 5′ 1, 2, 3, 4, or 5 nucleotides of the gRNA molecule (and in the case of a dgRNA molecule, the polynucleotide comprising the targeting domain and/or the polynucleotide comprising the tracr) are modified nucleic acids, as described more fully below.

The domains are discussed briefly below:

Guidance on the selection of targeting domains can be found in, e.g., Fu Y el al. NAT BIOTECHNOL (doi: 10.1038/nbt.2808) (2014) and Sternberg S H el al. NATURE (doi: 10.1038/naturel3011) (2014).

The targeting domain comprises a nucleotide sequence that is complementary, e.g., at least 80, 85, 90, 95, or 99% complementary, or e.g., fully complementary, to the target sequence on the target nucleic acid. The targeting domain is part of an RNA molecule and will therefore comprise the base uracil (U), while any DNA encoding the gRNA molecule will comprise the base thymine (T). While not wishing to be bound by theory, it is believed that the complementarity of the targeting domain with the target sequence contributes to specificity of the interaction of the gRNA molecule/Cas9 molecule complex with a target nucleic acid. It is understood that in a targeting domain and target sequence pair, the uracil bases in the targeting domain will pair with the adenine bases in the target sequence.

In an embodiment, the targeting domain is 5 to 50, e.g., 10 to 40, e.g., 10 to 30, e.g., 15 to 30, e.g., 15 to 25 nucleotides in length. In an embodiment, the targeting domain is 15, 16, 17, 18, 19, 20, 21, 22, 23, 24 or 25 nucleotides in length. In an embodiment, the targeting domain is 16 nucleotides in length. In an embodiment, the targeting domain is 17 nucleotides in length. In an embodiment, the targeting domain is 18 nucleotides in length. In an embodiment, the targeting domain is 19 nucleotides in length. In an embodiment, the targeting domain is 20 nucleotides in length. In an embodiment, the targeting domain is 21 nucleotides in length. In an embodiment, the targeting domain is 22 nucleotides in length. In an embodiment, the targeting domain is 23 nucleotides in length. In an embodiment, the targeting domain is 24 nucleotides in length. In an embodiment, the targeting domain is 25 nucleotides in length. In embodiments, the aforementioned 16, 17, 18, 19, or 20 nucleotides comprise the 5′-16, 17, 18, 19 or 20 nucleotides from a targeting domain described in Table 2. In embodiments, the aforementioned 16, 17, 18, 19, or 20 nucleotides comprise the 3′-16, 17, 18, 19 or 20 nucleotides from a targeting domain. In embodiments, the aforementioned 16, 17, 18, 19, or 20 nucleotides consist of the 3′-16, 17, 18, 19 or 20 nucleotides from a targeting domain.

In some embodiments, the Cas molecule in the gRNA/Cas complex is a Class 1 Cas nuclease. In some embodiments, the Cas molecule is a Class 2 Cas nuclease. See, e.g., Makarova et al. Nat Rev Microbiol, 13 (11): 722-36 (2015); Shmakov et al. Molecular Cell, 60:385-397 (2015). A Class 2 Cas molecule may be a single-protein endonuclease. In some embodiments, the Class 2 Cas molecule is from a Type II, V, or VI CRISPR/Cas system and may be a single-protein endonuclease. Non-limiting examples of Class 2 Cas molecules include Cas9, Cpf1, C2c1, C2c2, and C2c3 proteins. See, e.g., Yang et al. Cell, 167 (7): 1814-28 (2016); Zetsche et al. Cell, 163:1-13 (2015). In some embodiments, the Cas molecule is a S. aureus Cas9 molecule.

In some embodiments, the Cas molecule is a Cas9 molecule or fragment or variant, e.g., non-catalytic variant, thereof. Cas9 molecules of a variety of species can be used in the methods and compositions described herein. While the S. aureus Cas9 molecule are the subject of much of the disclosure herein, Cas9 molecules of, derived from, or based on the Cas9 proteins of other species listed herein can be used as well. In other words, other Cas9 molecules, e.g., S. thermophilus, Staphylococcus pyrogenes and/or Neisseria meningitidis Cas9 molecules, may be used in the systems, methods and compositions described herein.

In some embodiments, the Cas9 molecule is a high-fidelity variant harboring alterations designed to reduce non-specific DNA contacts. See, e.g., Kleinstiver et al. Nature 529 (7587): 490-95 (2016); Slaymaker et al. Science, 351 (6268): 84-88 (2016); Tsai et al. Nat. Biotech. 32:569-577 (2014). In some embodiments, the high-fidelity Cas9 retains on-target activities comparable to wild-type Cas9. In some embodiments, the high-fidelity Cas9 reduces off-target activities by at least about 50%, 60%, 70%, 80%, 90%, 95%, or 99% as compared to wild-type Cas9, e.g., as measured by genome-wide break capture and targeted sequencing methods. In some embodiments, the high-fidelity Cas9 renders off-target activities undetectable, e.g., as measured by genome-wide break capture and targeted sequencing methods. In some embodiments, the high-fidelity Cas9 is Streptococcus aureus Cas9.

Additional Cas9 species include: Acidovorax avenae, Actinobacillus pleuropneumoniae, Actinobacillus succinogenes, Actinobacillus suis, Actinomyces sp., cycliphilus denitrificans, Aminomonas paucivorans, Bacillus cereus, Bacillus smithii, Bacillus thuringiensis, Bacteroides sp., Blastopirellula marina, Bradyrhiz′ obium sp., Brevibacillus latemsporus, Campylobacter coli, Campylobacter jejuni, Campylobacter lad, Candidatus Puniceispirillum, Clostridiu cellulolyticum, Clostridium perfringens, Corynebacterium accolens, Corynebacterium diphtheria, Corynebacterium matruchotii, Dinoroseobacter sliibae, Eubacterium dolichum, gamma proteobacterium, Gluconacetobacler diazotrophicus, Haemophilus parainfluenzae, Haemophilus sputorum, Helicobacter canadensis, Helicobacter cinaedi, Helicobacter mustelae, Ilyobacler polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica. Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tislrella mobilis, Treponema sp., or Verminephrobacter eiseniae.

A Cas9 molecule, as that term is used herein, refers to a molecule that can interact with a gRNA molecule (e.g., sequence of a domain of a tracr) and, in concert with the gRNA molecule, localize (e.g., target or home) to a site which comprises a target sequence and PAM sequence.

In an embodiment, the ability of an active Cas9 molecule to interact with a target nucleic acid is PAM sequence dependent. A PAM sequence is a sequence in the target nucleic acid. Active Cas9 molecules from different bacterial species can recognize different sequence motifs (e.g., PAM sequences). In an embodiment, a Cas9 molecule of S. aureus recognizes the sequence motif NGRR (R=A or G) and directs cleavage of a target nucleic acid sequence 1 to 10, e.g., 3 to 5, base pairs upstream from that sequence. See, e.g., Ran F. et al., NATURE 520:186-191 (2015). The ability of a Cas9 molecule to recognize a PAM sequence can be determined, e.g., using a transformation assay described in Jinek et al, SCIENCE 337:816 (2012). Some Cas9 molecules have the ability to interact with a gRNA molecule, and in conjunction with the gRNA molecule home (e.g., targeted or localized) to a core target domain, but are incapable of cleaving the target nucleic acid, or incapable of cleaving at efficient rates. Cas9 molecules having no, or no substantial, cleavage activity may be referred to herein as an inactive Cas9 (an enzymatically inactive Cas9), a dead Cas9, or a dCas9 molecule. For example, an inactive Cas9 molecule can lack cleavage activity or have substantially less, e.g., less than 20, 10, 5, 1 or 0.1% of the cleavage activity of a reference Cas9 molecule, as measured by an assay described herein.

Exemplary naturally occurring Cas9 molecules that may be used with the methods provided herein are described in Chylinski et al., RNA Biology 10 (5): 727-737 (2013). Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 1 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

Exemplary naturally occurring Cas9 molecules include a Cas9 molecule of a cluster 1 bacterial family. Examples include a Cas9 molecule of: S. pyogenes (e.g., strain SF370, MGAS 10270, MGAS 10750, MGAS2096, MGAS315, MGAS5005, MGAS6180, MGAS9429, NZ131 and SSI-1), S. thermophilus (e.g., strain LMD-9), S. pseudoporcinus (e.g., strain SPIN 20026), S. mutans (e.g., strain UA 159, NN2025), S. macacae (e.g., strain NCTC1 1558), S. gallolylicus (e.g., strain UCN34, ATCC BAA-2069), S. equines (e.g., strain ATCC 9812, MGCS 124), S. dysdalactiae (e.g., strain GGS 124), S. bovis (e.g., strain ATCC 700338), S. anginosus (e.g., strain F021 1), S. agalactia (e.g., strain NEM316, A909), Listeria monocytogenes (e.g., strain F6854), Listeria innocua (e.g., strain Clip I 1262), Enterococcus italicus (e.g., strain DSM 15952), Enterococcus faecium (e.g., strain 1,231,408), C. jejune, or Deltaproteobacteria (Dbp). Additional exemplary Cas9 molecules are a Cas9 molecule of Neisseria meningitidis (Hou et al. PNAS Early Edition 1-6 (2013)) and a S. aureus Cas9 molecule.

In an embodiment, a Cas9 molecule, e.g., an inactive Cas9 molecule, comprises an amino acid sequence: having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; any Cas9 molecule sequence described herein or a naturally occurring Cas9 molecule sequence, e.g., a Cas9 molecule from a species listed herein or described in Chylinski et al., RNA Biology 10:5 (2013), or Hou et al. PNAS Early Edition 1-6 (2013).

In an embodiment, a Cas9 molecule comprises an amino acid sequence having 60%, 65%, 70%, 75%, 80%, 85%, 90%, 95%, 96%, 97%, 98%, or 99% homology with; differs at no more than 1%, 2%, 5%, 10%, 15%, 20%, 30%, or 40% of the amino acid residues when compared with; differs by at least 1, 2, 5, 10 or 20 amino acids but by no more than 100, 80, 70, 60, 50, 40 or 30 amino acids from; or is identical to; S. aureus Cas9.

Various types of Cas molecules can be used herein. In some embodiments, Cas molecules of Type II Cas systems are used. In other embodiments, Cas molecules of other Cas systems are used. For example, Type I or Type III Cas molecules may be used. Exemplary Cas molecules (and Cas systems) are described in, e.g., Haft et al., PLOS COMPUTATIONAL BIOLOGY 1 (6): e60 (2005) and Makarova et al., NATURE REVIEW MICROBIOLOGY 9:467-477 (2011), the contents of both references are incorporated herein by reference in their entirety.

### Altered Cas9 Molecules

Naturally occurring Cas9 molecules may possess a number of properties, including: nickase activity, nuclease activity (e.g., endonuclease and/or exonuclease activity); helicase activity; the ability to associate functionally with a gRNA molecule; and the ability to target (or localize to) a site on a nucleic acid (e.g., PAM recognition and specificity). In an embodiment, a Cas9 molecule used with the methods disclosed herein can include all or a subset of these properties. In typical embodiments, Cas9 molecules have the ability to interact with a gRNA molecule and, in concert with the gRNA molecule, localize to a site in a nucleic acid. Other activities, e.g., PAM specificity, cleavage activity, or helicase activity can vary more widely in Cas9 molecules.

Cas9 molecules with desired properties can be made in a number of ways, e.g., by alteration of a parental, e.g., naturally occurring Cas9 molecule to provide an altered Cas9 molecule having a desired property. For example, one or more mutations or differences relative to a parental Cas9 molecule can be introduced. Such mutations and differences may comprise: substitutions (e.g., conservative substitutions or substitutions of non-essential amino acids); insertions; or deletions. In an embodiment, a Cas9 molecule can comprises one or more mutations or differences, e.g., at least 1, 2, 3, 4, 5, 10, 15, 20, 30, 40 or 50 mutations but less than 300, 200, 100, or 80 mutations relative to a reference Cas9 molecule while retaining or enhancing one or more activities of the reference Cas9 molecule. For example, in one embodiment, the Cas molecule comprises a deletion of one or more amino acids as compared to the wild-type Cas molecule sequence.

In an embodiment, a mutation or mutations do not have a substantial effect on the Cas9 activity of targeting (or localizing) to a side on a nucleic acid (e.g., PAM recognition and specificity). In an embodiment, a mutation or mutations have a substantial effect on a Cas9 activity, e.g., the Cas9 activities of nickase activity, nuclease activity, and helicase activity.

Whether or not a particular sequence, e.g., a substitution, may affect one or more activity, such as targeting activity, cleavage activity, etc., can be evaluated or predicted by, e.g., evaluating whether the mutation is conservative or by the method described above. In an embodiment, a “non-essential” amino acid residue, as used in the context of a Cas9 molecule, is a residue that can be altered from the wild-type sequence of a Cas9 molecule, e.g., a naturally occurring Cas9 molecule, e.g., an active Cas9 molecule, without abolishing or more preferably, without substantially altering a Cas9 activity (e.g., targeting ability), whereas changing an “essential” amino acid residue results in a substantial loss of activity (e.g., targeting ability).

Cas9 Molecules with Altered PAM Recognition

Naturally occurring Cas9 molecules may recognize specific PAM sequences, for example the PAM recognition sequences described above for S. pyogenes, S. thermophilus, S. mutans, S. aureus and N. meningitidis.

In an embodiment, a Cas9 molecule has the same PAM specificities as a naturally occurring Cas9 molecule. In other embodiments, a Cas9 molecule has a PAM specificity not associated with a naturally occurring Cas9 molecule, or a PAM specificity not associated with the naturally occurring Cas9 molecule to which it has the closest sequence homology. For example, a naturally occurring Cas9 molecule can be altered, e.g., to alter PAM recognition, e.g., to alter the PAM sequence that the Cas9 molecule recognizes to decrease off target sites and/or improve specificity; or eliminate a PAM recognition requirement. In an embodiment, a Cas9 molecule can be altered, e.g., to increase length of PAM recognition sequence and/or improve Cas9 specificity to high level of identity to decrease off target sites and increase specificity. In an embodiment, the length of the PAM recognition sequence is at least 4, 5, 6, 7, 8, 9, 10 or 15 amino acids in length. Cas9 molecules that recognize different PAM sequences and/or have reduced off-target activity can be generated using directed evolution. Exemplary methods and systems that can be used for directed evolution of Cas9 molecules are described in, e.g., Esvelt el al, Nature 472 (7344): 499-503 (2011). Candidate Cas9 molecules can be evaluated, e.g., by methods described herein.

### Non-Cleaving Cas9 Molecules

In an embodiment, a Cas9 molecule comprises a cleavage property that differs from a naturally occurring Cas9 molecule, e.g., that differs from the naturally occurring Cas9 molecule having the closest homology. For example, a Cas9 molecule can differ from naturally occurring Cas9 molecules, e.g., a Cas9 molecule of S. aureus, as follows: its ability to modulate, e.g., decreased, cleavage of a double stranded break (endonuclease and/or exonuclease activity), e.g., as compared to a naturally occurring Cas9 molecule (e.g., a Cas9 molecule of S. aureus); its ability to modulate, e.g., decreased cleavage of a single strand of a nucleic acid, e.g., a non-complementary strand of a nucleic acid molecule or a complementary strand of a nucleic acid molecule (nickase activity), e.g., as compared to a naturally occurring Cas9 molecule (e.g., a Cas9 molecule of S. aureus); or the ability to cleave a nucleic acid molecule, e.g., a double stranded or single stranded nucleic acid molecule, can be eliminated.

### Non-Cleaving Inactive Cas9 Molecules

In an embodiment, the altered Cas9 molecule is an inactive Cas9 molecule which does not cleave a nucleic acid molecule (either double stranded or single stranded nucleic acid molecules) or cleaves a nucleic acid molecule with significantly less efficiency, e.g., less than 20, 10, 5, 1 or 0.1% of the cleavage activity of a reference Cas9 molecule, e.g., as measured by an assay described herein. The reference Cas9 molecule can by a naturally occurring unmodified Cas9 molecule, e.g., a naturally occurring Cas9 molecule such as a Cas9 molecule of S. pyogenes, S. thermophilus, S. aureus or N. meningitidis. In an embodiment, the reference Cas9 molecule is the naturally occurring Cas9 molecule having the closest sequence identity or homology. In an embodiment, the inactive Cas9 molecule lacks substantial cleavage activity associated with an N-terminal RuvC-like domain and cleavage activity associated with an HNH-like domain. In an embodiment, the Cas9 molecule is dCas9. See, e.g., Tsai et al. Nat. Biotech. 32:569-577 (2014).

A catalytically inactive Cas9 molecule may be fused with a transcription activator. An inactive Cas9 fusion protein complexes with a gRNA and localizes to a DNA sequence specified by gRNA's targeting domain, but, unlike an active Cas9, it will not cleave the target DNA. Fusion of an effector domain, such as a transcriptional activation domain, to an inactive Cas9 enables recruitment of the effector to any DNA site specified by the gRNA. Site specific targeting of a Cas9 fusion protein to a promoter region of a gene can induce or affect polymerase binding to the promoter region, for example, a Cas9 fusion with a transcription factor (e.g., a transcription activator) and/or a transcriptional enhancer binding to the nucleic acid to increase transcription activation. In one embodiment of the invention, the transcriptional activator or domain thereof is encoded by a nucleic acid molecule comprising fewer than 1,650 nucleotides. In another embodiment of the invention, the transcription repressor or domain thereof is encoded by a nucleic acid molecule comprising fewer than 1,650 nucleotides.

TABLE 2
below, provides the sequences of gRNAs for SCN1A

| SEQ ID NO: | CUGAUUUGUAUUAGGUACCA | sgRNA3 |
|---|---|---|
| 65 | | |
| SEQ ID NO: | GACUUUACAUGAGGAAUUUU | sgRNA11 |
| 66 | | |
| SEQ ID NO: | AUAUGAGUUCAAAGUUAUUA | sgRNA42 |
| 67 | | |

### Zinc Finger Protein

In some embodiments, the DBD is a zinc finger protein. Zinc finger protein can be used interchangably with “zinc finger domain” and “zinc finger motif.” A zinc finger is a small protein structural motif that is characterized by the coordination of one or more zinc ions (Zn²⁺) in order to stabilize the fold. Zinc finger (Znf) domains are relatively small protein motifs that contain multiple finger-like protrusions that make tandem contacts with a DNA target site. The modular nature of the zinc finger motif allows for a large number of combinations of DNA sequences to be bound with high degree of affinity and specificity, and is therefore ideally suited for engineering protein that can be targeted to and bind specific DNA sequences. Many engineered zinc finger arrays are based on the zinc finger domain of the murine transcription factor Zif268. Zif268 has three individual zinc finger motifs that collectively bind a 9 bp sequence with high affinity. A wide variety of zinc fingers proteins have been identified and are characterized into different types based on structure as further described herein. Any such zinc finger protein is useful in connection with the DBDs described herein.

Various methods for designing zinc finger proteins are available. For example, methods for designing zinc finger proteins to bind to a target DNA sequence of interest are described, see e.g., Liu Q, et ah, Design of polydactyl zinc-finger proteins for unique addressing within complex genomes, Proc Natl Acad Sci USA. 94 (11): 5525-30 (1997); Wright D A et ah, Standardized reagents and protocols for engineering zinc finger nucleases by modular assembly, Nat Protoc. Nat Protoc. 2006; 1 (3): 1637-52; and CA Gersbach and T Gaj, Synthetic Zinc Finger Proteins: The Advent of Targeted Gene Regulation and Genome Modification Technologies, Am Chem Soc 47:2309-2318 (2014). In addition, various web based tools for designing zinc finger proteins to bind to a DNA target sequence of interest are publicly available, see e.g., the Zinc Finger Nuclease Design Software Tools and Genome Engineering Data Analysis website from OmicX available on the world wide web at omictools.com/zfns-category; and the Zinc Finger Tools design website from Scripps available on the world wide web at scripps.edu/barbas/zfdesign/zfdesignhome.php. In addition, various commercially available services for designing zinc finger proteins to bind to a DNA target sequence of interest are available, see e.g., the commercially available services or kits offered by Creative Biolabs (world wide web at creative-biolabs.com/Design-and-Synthesis-of-Artificial-Zinc-Finger-Proteins.html), the Zinc Finger Consortium Modular Assembly Kit available from Addgene (world wide web at addgene.org/kits/zfc-modular-assembly/), or the CompoZr Custom ZFN Service from Sigma Aldrich (world wide web at sigmaaldrich.com/life-science/zinc-fmger-nuclease-technology/custom-zfn.html).

In certain embodiments, the transcription factors provided herein that comprise a DBD comprising one or more zinc fingers or is derived from a DBD of a zinc finger protein. In some cases, the DBD comprises multiple zinc fingers, wherein each zinc finger is linked to another zinc finger or another domain either at its N-terminus or C-terminus, or both via an amino acid linker. In some cases, a DBD provided herein comprises a plurality of zinc finger structures or motifs, or a plurality of zinc fingers having one or more of SEQ ID NOs: 70-73, and 61 described in Table 3, or any combination thereof.

In certain embodiments, a DBD comprises X-[ZF-X]n and/or [X-ZF]n-X, wherein ZF is a zinc finger domain, X is an amino acid linker comprising 1-50 amino acids, and n is an integer from 1-15, e.g., 1, 2, 3, 4, 5, 6, 7, 8, 9, 10, 11, 12, 13, 14, or 15, wherein each ZF can independently have the same sequence or a different sequence from the other ZF sequences in the DBD, and wherein each linker X can independently have the same sequence or a different sequence from the other X sequences in the DBD. Each zinc finger can be linked to another sequence, zinc finger, or domain at its C-terminus, N-terminus, or both. In a DBD, each linker X can be identical in sequence, length, and/or property (e.g., flexibility or charge), or be different in sequence, length, and/or property. In some cases, two or more linkers may be identical, while other linkers are different. In exemplary embodiments, the linker may be obtained or derived from the sequences connecting the zinc fingers found in one or more naturally occurring zinc finger proteins. In other embodiments, suitable linker sequences, include, for example, linkers of 5 or more amino acids in length. See, also, U.S. Pat. Nos. 6,479,626; 6,903,185; and 7,153,949 for exemplary linker sequences of 6 or more amino acids in length, each of which is incorporated herein in their entireties. The DBD proteins provided herein may include any combination of suitable linkers between the individual zinc fingers of the protein. The DBD proteins described herein may include any combination of suitable linkers between the individual zinc fingers of the protein.

In certain embodiments, the transcription factors provided herein that comprise a DBD comprising one or more classic zinc fingers. A classical C2H2 zinc-finger has two cysteines in one chain and two histidine residues in another chain, coordinated by a zinc ion. A classical zinc-finger domain has two b-sheets and one a-helix, wherein the a-helix interacts with a DNA molecule and forms the basis of the DBD binding to a target site and may be referred to as the “recognition helix”. In exemplary embodiments, the recognition helix of a zinc fingers comprises at least one amino acid substitution at position-1, 2, 3 or 6 thereby changing the binding specificity of the zinc finger domain. In other embodiments, an DBD provided herein comprises one or more non-classical zinc-fingers, e.g., C2-H2, C2-CH, and C2-C2.

In another embodiment, an transcription factor provided herein comprises a DBD comprising a zinc finger motif having the following structure: LEPGEKP-[YKCPECGKSFS X HQRTH TGEKP]n-YKCPECGKSFS X HQRTH-TGKKTS, wherein “LEPGEKPYKCPECGKSFS” is disclosed as SEQ ID NO: 91, “HQRTHTGEKPYKCPECGKSFS” is disclosed as SEQ ID NO: 92, “HQRTHTGKKTS” is disclosed as SEQ ID NO: 83, and n is an integer from 1-15, e.g., 1, 2, 3, 4, 5, 6, 7, 8, 9, 10, 11, 12, 13, 14, or 15, and each X independently is a recognition sequence (e.g., a recognition helix) capable of binding to 3 bp of the target sequence. In exemplary embodiments, n is 3, 6 or 9. In a particularly preferred embodiment, n is 6. In various embodiments, each X may independently have the same amino acid sequence or a different amino acid sequence as compared to other X sequences in the DBD. In an exemplary embodiment, each X is a sequence comprising 7 amino acids that has been designed to interact with 3 bp of the target binding site of interest using the Zinger Finger Design Tool from Scripps located on world wide web at scripps.edu/barbas/zfdesign/zfdesignhome.php.

Since each zinc finger within a DBD recognizes 3 bp, the number of zinc fingers included in the DBD informs the length of the binding site recognized by the DBD, e.g., a DBD with 1 zinc finger will recognize a target binding site having 3 bp, a DBD with 2 zinc fingers will recognize a target binding site having 6 bp, a DBD with 3 zinc fingers will recognize a target binding site having 9 bp, a DBD with 4 zinc fingers will recognize a target binding site having 12 bp, a DBD with 5 zinc fingers will recognize a target binding site having 15 bp, a DBD with 6 zinc fingers will recognize a target binding site having 18 bp, a DBD with 9 zinc fingers will recognize a target binding site having 27 bp, etc. In general, DBD that recognize longer target binding sites will exhibit greater binding specificity (e.g., less off target or non-specific binding).

In other embodiments, the transcription factors provided herein comprise a DBD that is derived from a naturally occurring zinc finger protein by making one or more amino acid substitutions in one or more of the recognition helices of the zinc finger domains so as to change the binding specificity of the DBD (e.g., changing the target site recognized by the DBD). DBD provided herein may be derived from any naturally occurring zinc finger protein.

In various embodiments, such DBD may be derived from a zinc finger protein of any species, e.g., a mouse, rat, human, etc. In an exemplary embodiment, a DBD provided herein is derived from a human zinc finger protein. In certain embodiments, a DBD provided herein is derived from a naturally occurring protein listed in TABLE 9. In an exemplary embodiment, a DBD protein provided herein is derived from a human EGR zinc finger protein, e.g., EGR1, EGR2, EGR3, or EGR4.

In certain embodiments, a transcription factor provided herein that upregulates SCN1 A comprises a DBD that is derived from a naturally occurring protein by modifying the DBD to increase the number of zinc finger domains in the DBD protein by repeating one or more zinc fingers within the DBD of the naturally occurring protein. In certain embodiments, such modifications include duplication, triplication, quadruplication, or further multiplication of the zinc fingers within the DBD of the naturally occurring protein. In some cases, one zinc finger from a DBD of a human protein is multiplied, e.g., 2, 3, 4, 5, 6, 7, 8, 9, 10, 11, 12, or more copies of the same zinc finger motif is repeated in the DBD of the transcription factor. In some cases, a set of zinc fingers from a DBD of a naturally occurring protein is multiplied, e.g., a set of 3 zinc fingers from a DBD of a naturally occurring protein is duplicated to yield a transcription factor having a DBD with 6 zinc fingers, is triplicated to yield a DBD of a transcription factor with 9 zinc fingers, or is quadruplicated to yield a DBD of a transcription factor with 12 zinc fingers, etc. In some cases, a set of zinc fingers from a DBD of a naturally occurring protein is partially replicated to form a DBD of a transcription factor having a greater number of zinc fingers, e.g., a DBD of a transcription factor comprises four zinc fingers wherein the zinc fingers represent one copy of the first zinc finger, one copy of the second zinc finger, and two copies of a third zinc finger from a naturally occurring protein for a total of four zinc fingers in the DBD of the transcription factor. Such DBD are then further modified by making one or more amino acid substitutions in one or more of the recognition helices of the zinc finger domains so as to change the binding specificity of the DBD (e.g., changing the target site recognized by the DBD). In exemplary embodiments, the DBD is derived from a naturally occurring human protein, such as a human EGR zinc finger protein, e.g., EGR1, EGR2, EGR3, or EGR4.

Human EGR1 and EGR3 are characterized by a three-finger C2H2 zinc finger DBD. The generic binding rules for zinc fingers provide that all three fingers interact with its cognate DNA sequence with similar geometry, using the same amino acids in the alpha helix of each zinc finger to determine the specificity or recognition of the target binding site sequence. Such binding rules allow one to modify the DBD of EGR1 or EGR3 to engineer a DBD that recognizes a desired target binding site. In some cases, the 7-amino acid DNA recognition helix in a zinc finger motif of EGR1 or EGR3 is modified according to published zinc finger design rules. In certain embodiments, each zinc finger in the three-finger DBD of EGR1 or EGR3 is modified, e.g., by altering the sequence of one or more recognition helices and/or by increasing the number of zinc fingers in the DBD. In certain embodiments, EGR1 or EGR3 is reprogrammed to recognize a target binding site of at least 9, 12, 15, 18, 21, 24, 27, 30, 33, 36 or more base pairs at a desired target site. In certain embodiments, such DBD derived from ERG1 or EGR3 comprises at least 3, 4, 5, 6, 7, 8, 9, 10, 11, 12, or more zinc fingers. In exemplary embodiment, one or more of the zinc fingers in the DBD comprises at least one amino acid substitution at position-1, 2, 3 or 6 of the recognition helix.

TABLE 3
below, provides the amino acid sequences of
examplary zinc finger proteins used in the Examples

| | | |
|---|---|---|
| SEQ ID | MPKKKRKVGIHGVPAAMAERPFQCRICMRNFS | E2C |
| NO: 70 | RSDNLSEHIRTHTGEKPFACDICGRKFAKRCNL | (amino |
| | RCHTKIHTGSQKPFQCRICMRNFSRSDNLSEHI | acid |
| | RTHTGEKPFACDICGRKFAKRCNLRCHTKIHTG | sequence) |
| | SQKPFQCRICMRNFSRSDNLSEHIRTHTGEKPF | |
| | ACDICGRKFAKRCNLRCHTKIHLRQKDAAR | |
| SEQ ID | MPKKKRKVAQAALEPGEKPYACPECGKSFSRK | ZFP-C |
| NO: 71 | DSLVRHQRTHTGEKPYKCPECGKSFSQSGDL | (amino |
| | RRHQRTHTGEKPYKCPECGKSFSDCRDLARH | acid |
| | QRTHTGEKPYACPECGKSFSQSSHLVRHQRT | sequence) |
| | HTGEKPYKCPECGKSFSDCRDLARHQRTHTG | |
| | EKPYKCPECGKSFSRSDKLVRHQRTHTGKKTS | |
| | GQAG | |
| SEQ ID | CPRRKGKSHRQLSSLAKNLTHVRSVGSLSQHLIT | Nav-ZF6 |
| NO: 73 | LSHIKEPTLVKSHINAQSVERVLASLTIW*DIRE | (amino |
| | PTLARSLTNAPNAENHSVRLITWSGIRELILVKN | acid |
| | HMHARSAANHFPHQTIL*PTRELTRGRSHTSAL | sequence) |
| | NVESHSQSQTHSCGTSARIRGRNRTNARNAG | |
| | SHSVAQITWSAINEPIPVRRQVAKL | |
| SEQ ID | MAQAALEPGEKPYACPECGKSFSRSTNLVRHQ | Nav-ZF2 |
| NO: 61 | RTHTGEKPYKCPECGKSFSECDDLARHQRTHT | (amino |
| | GEKPYKCPECGKSFSQSDNLVRHQRTHTGEK | acid |
| | PYACPECGKSFSTSDNLVRHQRTHTGEKPYKC | sequence) |
| | PECGKSFSRSDNLVRHQRTHTGEKPYKCPEC | |
| | GKSFSDRDHLVRHQRTHTGKKTSGQAG | |

TABLE 4
below, provides other sequences used
in the transcription factors

| | | |
|---|---|---|
| SEQ ID NO: | GGSGGGSG | Linker (amino |
| 59 | | acid sequence) |
| SEQ ID NO: | GGSGGGSGGGSGGGSG | Linker (amino |
| 60 | | acid sequence) |
| SEQ ID NO: | MAQAALEPGEKPYACPECGKSFSRSTNLVRHQ | Nav-ZF2 (amino |
| 61 | RTHTGEKPYKCPECGKSFSECDDLARHQRTHT | acid sequence) |
| | GEKPYKCPECGKSFSQSDNLVRHQRTHTGEK | |
| | PYACPECGKSFSTSDNLVRHQRTHTGEKPYKC | |
| | PECGKSFSRSDNLVRHQRTHTGEKPYKCPEC | |
| | GKSFSDRDHLVRHQRTHTGKKTSGQAG | |
| SEQ ID NO: | ATGCCCAAGAAGAAAAGGAAAGTCGGAGGTG | Mini-dSaCas9 |
| 62 | GAAGCCCGGGTAAACGCAATTATATCCTGGG | (nucleic acid |
| | GTTGGCCATCGGAATCACTAGTGTTGGATAT | sequence) |
| | GGGATTATTGACTACGAGACGCGCGATGTGA | |
| | TCGATGCCGGAGTACGGCTTTTCAAGGAGGC | |
| | AAACGTCGAGAACAATGAGGGGCGGCGGTC | |
| | TAAAAGAGGAGCACGAAGGCTCAAGAGGCG | |
| | AAGAAGGCATCGGATTCAACGCGTCAAGAAA | |
| | TTGCTCTTCGATTACAATCTGCTGACAGACCA | |
| | CTCCGAGTTGTCCGGAATAAATCCGTACGAG | |
| | GCGAGAGTCAAAGGCCTCAGTCAGAAGCTCT | |
| | CCGAAGAAGAGTTCAGCGCTGCACTTCTCCA | |
| | CCTTGCGAAACGAAGAGGAGTGCATAACGTA | |
| | AACGAAGTCGAAGAGGATACAGGCAACGAG | |
| | CTGTCTACGAAAGAGCAGATTAGTAGGAATA | |
| | GCAAGGCACTCGAAGAGAAATATGTGGCGGA | |
| | ACTCCAACTCGAAAGGCTCAAGAAGGACGGT | |
| | GAAGTCAGGGGTTCTATAAATCGGTTTAAAAC | |
| | ATCCGACTATGTCAAAGAAGCGAAGCAACTT | |
| | CTTAAGGTCCAAAAGGCATACCACCAGTTGG | |
| | ATCAATCATTCATTGACACTTACATTGACCTG | |
| | CTCGAGACACGACGGACTTACTACGAAGGAC | |
| | CGGGAGAAGGGAGTCCGTTCGGCTGGAAGG | |
| | ACATTAAGGAATGGTATGAGATGCTCATGGG | |
| | ACATTGTACGTATTTCCCCGAAGAACTGCGAT | |
| | CAGTCAAGTATGCTTATAATGCAGATTTGTAC | |
| | AACGCATTGAATGATTTGAATAATCTGGTCAT | |
| | CACACGAGACGAAAACGAAAAATTGGAGTAC | |
| | TACGAAAAATTCCAGATTATCGAAAATGTCTT | |
| | TAAACAGAAGAAGAAGCCGACTCTCAAGCAA | |
| | ATAGCTAAAGAGATTCTTGTCAATGAAGAAGA | |
| | CATTAAAGGTTACCGAGTTACTAGCACTGGC | |
| | AAACCCGAGTTTACTAACCTGAAAGTGTACCA | |
| | CGATATCAAGGACATCACCGCGCGAAAAGAG | |
| | ATAATCGAAAACGCCGAACTTCTGGACCAAA | |
| | TTGCGAAAATTCTGACCATTTATCAATCCAGT | |
| | GAAGATATACAAGAAGAGCTGACGAACCTTA | |
| | ACTCAGAGCTTACGCAAGAGGAGATTGAGCA | |
| | AATAAGCAATCTGAAGGGTTATACGGGGACC | |
| | CACAACCTGAGTCTCAAAGCAATTAACCTTAT | |
| | CCTTGACGAGCTTTGGCATACAAACGACAAC | |
| | CAGATAGCTATATTTAACCGGCTCAAACTGGT | |
| | GCCGAAAAAAGTCGATCTTTCTCAGCAAAAA | |
| | GAAATTCCTACGACGCTCGTGGATGACTTTAT | |
| | TTTGTCACCCGTCGTAAAACGCTCCTTCATCC | |
| | AATCAATCAAAGTCATCAATGCGATAATAAAG | |
| | AAGTACGGCCTCCCGAACGATATAATTATAG | |
| | AGCTCGGTTCCAAGCGCTATGCCACGCGCG | |
| | GTTTGATGAACCTTCTCCGATCTTACTTCCGG | |
| | GTCAATAACCTCGACGTGAAGGTAAAATCAAT | |
| | TAATGGAGGGTTCACATCATTCCTCCGACGA | |
| | AAATGGAAGTTTAAAAAGGAACGCAACAAGG | |
| | GGTATAAGCATCATGCAGAGGATGCACTTAT | |
| | CATAGCCAACGCAGACTTTATTTTCAAGGAGT | |
| | GGAAAAAATTGGATAAAGCTAAGAAAGTCAT | |
| | GGAAAACCAGATGTTCGAAGAGAAACAAGCC | |
| | GAAAGCATGCCCGAGATCGAGACTGAGCAG | |
| | GAATATAAAGAAATCTTCATCACCCCGCATCA | |
| | AATAAAGCACATAAAAGACTTTAAAGATTACA | |
| | AGTATTCCCATCGGGTGGACAAAAAGCCTAA | |
| | TCGGAAGCTCATCAATGACACTCTTTACTCCA | |
| | CTAGAAAAGACGATAAGGGCAATACCCTCAT | |
| | AGTTAATAACCTGAATGGTTTGTACGATAAAG | |
| | ACAACGATAAGCTGAAAAAACTTATAAACAAG | |
| | TCCCCGGAAAAGTTGCTTATGTATCACCACG | |
| | ACCCGCAGACGTACCAGAAACTTAAGCTTAT | |
| | TATGGAACAGTATGGGGATGAGAAAAACCCA | |
| | CTCTACAAATACTACGAGGAAACGGGTAATTA | |
| | TTTGACTAAGTACTCAAAGAAAGATAATGGTC | |
| | CTGTGATAAAAAAGATAAAGTATTACGGGAAT | |
| | AAACTCAACGCTCACCTCGACATAACAGATG | |
| | ATTACCCTAATAGTCGAAATAAAGTCGTTAAG | |
| | TTGTCTCTTAAGCCGTATCGATTTGACGTGTA | |
| | TCTGGACAATGGCGTGTATAAGTTCGTCACG | |
| | GTTAAAAATCTTGACGTAATCAAAAAAGAAAA | |
| | TTATTATGAGGTAAACTCCAAATGTTACGAAG | |
| | AGGCGAAAAAACTCAAAAAAATATCAAATCAG | |
| | GCCGAGTTCATTGCGTCCTTTTATAAAAATGA | |
| | CCTGATAAAGATTAATGGAGAATTGTATCGG | |
| | GTGATAGGGGTGAACAATGACCTTTTGAATA | |
| | GAATTGAAGTTAATATGATCGACATAACCTAT | |
| | CGAGAGTATCTTGAAAATATGAACGACAAGA | |
| | GACCGCCACACATCATTAAAACTATCGCAAG | |
| | TAAGACCCAGTCAATCAAAAAATATAGCACAG | |
| | ATATACTTGGCAATCTTTACGAGGTCAAGTCT | |
| | AAGAAACACCCTCAGATTATTAAGAAAGGGA | |
| | CTTCTCGCGCGGAC | |
| SEQ ID NO: | MPKKKRKVGGGSPGKRNYILGLAIGITSVGYGII | Mini-dSaCas9 |
| 63 | DYETRDVIDAGVRLFKEANVENNEGRRSKRGA | (amino acid |
| | RRLKRRRRHRIQRVKKLLFDYNLLTDHSELSGI | sequence) |
| | NPYEARVKGLSQKLSEEEFSAALLHLAKRRGV | |
| | HNVNEVEEDTGNELSTKEQISRNSKALEEKYVA | |
| | ELQLERLKKDGEVRGSINRFKTSDYVKEAKQLL | |
| | KVQKAYHQLDQSFIDTYIDLLETRRTYYEGPGE | |
| | GSPFGWKDIKEWYEMLMGHCTYFPEELRSVK | |
| | YAYNADLYNALNDLNNLVITRDENEKLEYYEKF | |
| | QIIENVFKQKKKPTLKQIAKEILVNEEDIKGYRVT | |
| | STGKPEFTNLKVYHDIKDITARKEIIENAELLDQI | |
| | AKILTIYQSSEDIQEELTNLNSELTQEEIEQISNL | |
| | KGYTGTHNLSLKAINLILDELWHTNDNQIAIFNR | |
| | LKLVPKKVDLSQQKEIPTTLVDDFILSPVVKRSFI | |
| | QSIKVINAIIKKYGLPNDIIIELGSKRYATRGLMNL | |
| | LRSYFRVNNLDVKVKSINGGFTSFLRRKWKFKK | |
| | ERNKGYKHHAEDALIIANADFIFKEWKKLDKAK | |
| | KVMENQMFEEKQAESMPEIETEQEYKEIFITPH | |
| | QIKHIKDFKDYKYSHRVDKKPNRKLINDTLYSTR | |
| | KDDKGNTLIVNNLNGLYDKDNDKLKKLINKSPE | |
| | KLLMYHHDPQTYQKLKLIMEQYGDEKNPLYKY | |
| | YEETGNYLTKYSKKDNGPVIKKIKYYGNKLNAH | |
| | LDITDDYPNSRNKVVKLSLKPYRFDVYLDNGVY | |
| | KFVTVKNLDVIKKENYYEVNSKCYEEAKKLKKIS | |
| | NQAEFIASFYKNDLIKINGELYRVIGVNNDLLNRI | |
| | EVNMIDITYREYLENMNDKRPPHIIKTIASKTQSI | |
| | KKYSTDILGNLYEVKSKKHPQIIKKGTSRAD | |
| SEQ ID NO: | UACGUUCUCUAUCACUGAUA | TRE sgRNA |
| 64 | | |
| SEQ ID NO: | CUGAUUUGUAUUAGGUACCA | sgRNA3 |
| 65 | | |
| SEQ ID NO: | GACUUUACAUGAGGAAUUUU | sgRNA11 |
| 66 | | |
| SEQ ID NO: | AUAUGAGUUCAAAGUUAUUA | sgRNA42 |
| 67 | | |
| SEQ ID NO: | TGCCUCCCUGUCCUGUAGUA | sgRNA24 |
| 68 | | |
| SEQ ID NO: | CAUACAAUGAGAAUUGCUAG | sgRNA25 |
| 69 | | |
| SEQ ID NO: | MPKKKRKVGIHGVPAAMAERPFQCRICMRNFS | ZFP-C (amino |
| 70 | RSDNLSEHIRTHTGEKPFACDICGRKFAKRCNL | acid sequence) |
| | RCHTKIHTGSQKPFQCRICMRNFSRSDNLSEHI | |
| | RTHTGEKPFACDICGRKFAKRCNLRCHTKIHTG | |
| | SQKPFQCRICMRNFSRSDNLSEHIRTHTGEKPF | |
| | ACDICGRKFAKRCNLRCHTKIHLRQKDAAR | |
| SEQ ID NO: | MPKKKRKVAQAALEPGEKPYACPECGKSFSRK | E2C ZFP (amino |
| 71 | DSLVRHQRTHTGEKPYKCPECGKSFSQSGDL | acid sequence) |
| | RRHQRTHTGEKPYKCPECGKSFSDCRDLARH | |
| | QRTHTGEKPYACPECGKSFSQSSHLVRHQRT | |
| | HTGEKPYKCPECGKSFSDCRDLARHQRTHTG | |
| | EKPYKCPECGKSFSRSDKLVRHQRTHTGKKTS | |
| | GQAG | |
| SEQ ID NO: | GGGGCCGGAGCCGCAGTG | E2C Target Seq |
| 72 | | |
| SEQ ID NO: | CPRRKGKSHRQLSSLAKNLTHVRSVGSLSQHLI | Nav-ZF6 (amino |
| 73 | TLSHIKEPTLVKSHINAQSVERVLASLTIW*DIRE | acid sequence) |
| | PTLARSLTNAPNAENHSVRLITWSGIRELILVKN | |
| | HMHARSAANHFPHQTIL*PTRELTRGRSHTSAL | |
| | NVESHSQSQTHSCGTSARIRGRNRTNARNAG | |
| | SHSVAQITWSAINEPIPVRRQVAKL | |

### Upregulation of Gene of Interest

The transcription factors provided herein can be designed to recognize any target site (e.g. promoter region of a gene of interest) that results in upregulation of the gene of interest. In exemplary embodiments, a DBD is designed to recognize a genomic location and upregulate expression of an endogenous gene of interest when bound by a transcription factor. Binding sites capable of modulating expression of an endogenous gene of interest when bound by an transcription factor provided herein may be located anywhere in the genome that results in modulation of gene expression of the gene of interest. In various embodiments, the binding site may be located on a different chromosome from the gene of interest, on the same chromosome as the gene of interest, upstream of the transcriptional start site (TSS) of the gene of interest, downstream of the TSS of the gene of interest, proximal to the TSS of the gene of interest, distal to the gene of interest, within the coding region of the gene of interest, within an intron of the gene of interest, downstream of the poly A tail of the gene of interest, within a promoter sequence that regulates the gene of interest, or within an enhancer sequence that regulates the gene of interest.

The DBD may be designed to bind to a target binding site of any length so long as it provides specific recognition of the target binding site sequence by the DBD, e.g., with minimal or no off target binding. In certain embodiments, the target binding site may modulate expression of a gene of interest when bound by a transaction factor at a level that is at least 2-fold, 5-fold, 10-fold, 20-fold, 50-fold, 75-fold, 100-fold, 150-fold, 200-fold, 250-fold, 500-fold, or greater as compared to all other genes. In certain embodiments, the target binding site may modulate expression of a gene of interest when bound by a transaction factor at a level that is at least 2-fold, 5-fold, 10-fold, 20-fold, 50-fold, 75-fold, 100-fold, 150-fold, 200-fold, 250-fold, 500-fold, or greater as compared to the 40 nearest neighbor genes (e.g., the 40 genes located closest on the chromosome, either upstream or downstream, of the coding sequence of the gene of interest). In certain embodiments, the target binding site may be at least 5 bp, 10 bp, 15 bp, 20 bp, 25 bp, 30 bp, 35 bp, 40 bp, 45 bp or 50 bp, or more. The specific length of the binding site will be informed by the type of DBD in the transcription factor. In general, the longer the length of the binding site, the greater the specificity for binding and modulation of gene expression (e.g., longer binding sites have fewer off target effects). In certain embodiments, a transcription factor having a DBD recognizing a longer target binding site has fewer off-target effects associated with non specific binding (such as, for example, modulation of expression of an off-target gene or gene other than the gene of interest) relative to the off-target effects observed with a transcription factor having a DBD that binds to a shorter target site. In some cases, the reduction in off-target binding is at least 1.2, 1.3, 1.4, 1.5, 2, 3, 4, 5, 6, 7, 8, 9, or 10 fold lower as compared to a comparable transcription factor having a DBD that recognizes a shorter target binding site.

In certain embodiments, a DBD provided herein can be modified to have increased binding affinity such that it binds to a target binding site for a longer period of time such that a TAD conjugated to the DBD is able to recruit more transcription factors and/or recruit such transcription factor for a longer period of time to exert a greater effect on the expression level of the endogenous gene of interest. In certain embodiments, a DBD may be modified to increase its specific binding (or on-target binding) to a desired target site and/or modified to decrease its non-specific or off-target binding.

In various embodiments, binding between a DBD or transcription factor and a target binding site may be determined using various methods. In certain embodiments, specific binding between a DBD or transcription factor and a target binding site may be determined using a mobility shift assay, DNase protection assay, or any other in vitro method known in the art for assaying protein-DNA binding. In other embodiments, specific binding between transcription factor and a target binding site may be determined using a functional assay, e.g., by measuring expression (RNA or protein) of a gene of interest when the target binding site is bound by the transcription factor. For example, a target binding site may be positioned upstream of a reporter gene (such as, for example, eGFP) or a gene of interest on a vector contained in a cell or integrated into the genome of the cell, wherein the cell expresses transcription factor. Alternatively, a vector expressing the transcription factor may be introduced into a cell type that naturally contains the gene of interest. Greater levels of expression of the reporter gene (or gene of interest) in the presence of the transcription factor as compared to a control (e.g., no transcription factor or transcription factor that recognizes a different target site) indicate that the DBD of the transcription factor binds to the target site. Suitable in vitro (e.g., non cell based) transcriptional and translational systems may also be used in a similar manner. In certain embodiments, transcription factor that binds to a target site may have at least 2-fold, 3-fold, 5-fold, 10-fold, 15-fold, 20-fold, 30-fold, 50-fold, 75-fold, 100-fold, 150-fold, or greater expression of the reporter gene or gene of interest as compared to a control (e.g., no transcription factor or transcription factor that recognizes a different target site).

In certain embodiments, a transcription factor disclosed herein that upregulates gene of interest recognizes a target binding site that is at least 9 bp, 12 bp, 15 bp, 18 bp, 21 bp, 24 bp, 27 bp, 30 bp, 33 bp, or 36 bp in size; more than 9 bp, 12 bp, 15 bp, 18 bp, 21 bp, 24 bp, 27 bp, or 30 bp; or from 9-33 bp, 9-30 bp, 9-27 bp, 9-24 bp, 9-21 bp, 9-18 bp, 9-15 bp, 9-12 bp, 12-33 bp, 12-30 bp, 12-27 bp, 12-24 bp, 12-21 bp, 12-18 bp, 12-15 bp, 15-33 bp, 15-30 bp, 15-27 bp, 15-24 bp, 15-21 bp, 15-18 bp, 18-33 bp, 18-30 bp, 18-27 bp, 18-24 bp, 18-21 bp, 21-33 bp, 21-30 bp, 21-27 bp, 21-24 bp, 24-33 bp, 24-30 bp, 24-27 bp, 27-33 bp, 27-30 bp, or 30-33 bp. In exemplary embodiments, a transcription factor disclosed herein that upregulates gene of interest recognizes a target binding site that is 18-27 bp, 18 bp, or 27 bp.

In certain embodiments, a transcription factor disclosed herein that upregulates a gene of interest recognizes a target binding site that is located on chromosome 2. In certain embodiments, a transcription factor disclosed herein that upregulates a gene of interest recognizes a target binding site that is located on chromosome within 110 kb, 100 kb, 90 kb, 80 kb, 70 kb, 60 kb, 50 kb, 40 kb, 30 kb, 20 kb, 10 kb, 5 kb, 4 kb, 3 kb, 2 kb, or 1 kb upstream or downstream of the TSS of the gene of interest A.

Gene of interest may be any gene the genomic alternations of whom results in reduced transcription or activity of one or more gene or gene products are a causative factor in mammalian diseases, for example, the genomic alteration might be haploinsufficiency, in which there is only one functional copy of a gene and that single copy does not produce enough of the gene product to produce a wild-type phenotype. Other diseases are caused by genomic alterations in one or both copies of a gene that alter the gene product so that it exhibits a reduction, but not elimination, in activity. In still other diseases, genomic alterations reduce transcription or reduce transcript stability of one or both copies of a gene, such that there is insufficient gene product to produce a wild-type phenotype.

In some embodiments, the increase in expression of the gene of interest is measured by an increase in the number of RNA transcripts of the transgene sequence. In some embodiments, the increase in expression of the transgene sequence is measured by PCR. In some embodiments, the increase in expression of the transgene sequence is measured by RT-PCR. In some embodiments, the increase in expression of the transgene sequence is measured by qPCR. In some embodiments, the increase in expression of the transgene sequence is measured by qRT-PCR. In some embodiments, the increase in expression of the transgene sequence is measured by sequencing. In some embodiments, the increase in expression of the transgene sequence is measured by Northern blot analysis. In some embodiments, the increase in expression of the transgene sequence is measured by single-molecule Fluorescence In-Situ Hybridization (FISH). In some embodiments, the increase in expression of the transgene sequence is measured by an increase in the amount of protein encoded by the transgene produced. In some embodiments, the increase in expression of the transgene sequence is measured by an enzyme-linked immunosorbent assay (ELISA). In some embodiments, the increase in expression of the transgene sequence is measured by Western blot analysis. In some embodiments, the increase in expression of the transgene sequence is measured by immunostaining. In some embodiments, the increase in expression of the transgene sequence is measured by more than one of the above listed methods.

### Promoters

All cells in the animal or human body contain the same DNA, yet different cells in different tissues express, on the one hand, a set of common genes, and on the other, a set of genes that vary depending on the type of tissue and the stage of development. Without being bound by theory, any promoter that does not contain an intron can be used in the various aspects and embodiments (e.g., in the nucleic acid molecules) described herein. Exemplary promoters that can be used with the various aspects and embodiments described herein include, but are not limited to, a promoter operable in GABAnergic neurons, a promoter operable in inhibitory neurons, the cytomegalovirus (CMV) promoter, the CAG promoter, the SV40 promoter, the JeT promoter, the PGK promoter and the chicken beta-actin promoter (CBA) promoter. In embodiments, the promoter is active in more than one cell type. In other embodiments, the promoter is active in one cell type (e.g., cell-specific) or in cell types of one tissue (e.g., tissue-specific), such as, for example, central nervous tissue (e.g., brain tissue). In embodiments, the promoter is neuron specific. Examples of neuron specific promoters that can be used in the various aspects and embodiments described herein include, but are not limited to, isolated or synthetic neuron-specific promoters and functional fragments thereof used in vectors and other nucleic acids to drive expression of an operatively linked minigene and transgene, e.g., promoters derived from neuron-specific enolase (NSE) (see, e.g., EMBL HSENO2, X51956); an aromatic amino acid decarboxylase (AADC) promoter; a neurofilament promoter (see, e.g., GenBank HUMNFL, L04147); a synapsin promoter (see, e.g., GenBank HUMSYNIB, M55301); a thy-1 promoter (see, e.g., Chen et al., (1987) Cell, 51:7-19; Llewellyn et al. (2010) Nat. Med., 16 (10): 1161-1166); a serotonin receptor promoter (see, e.g., GenBank S62283); a tyrosine hydroxylase promoter (TH) (see, e.g., Oh et al., (2009) Gene Ther., 16:437; Sasaoka et al., (1992) Mol. Brain Res., 16:274; Boundy et al., (1998) J. Neurosci., 18:9989; and Kaneda et al., (1991) Neuron, 6:583-594); a GnRH promoter (see, e.g., Radovick et al., (1991) Proc. Natl. Acad. Sci. USA, 88:3402-3406); an L7 promoter (see, e.g., Oberdick et al., (1990) Science, 248:223-226); a DNMT promoter (see, e.g., Bartge et al., (1988) Proc. Natl. Acad. Sci. USA, 85:3648-3652); an enkephalin promoter (see, e.g., Comb et al., (1988) EMBO J., 17:3793-3805); a myelin basic protein (MBP) promoter; a Ca2+-calmodulin-dependent protein kinase II-alpha (CamKIM) promoter (see, e.g., Mayford et al., (1996) Proc. Natl. Acad. Sci. USA, 93:13250; and Casanova et al., (2001) Genesis, 31:37); a CMV enhancer/platelet-derived growth factor-p promoter (see, e.g., Liu et al., (2004) Gene Ther., 11:52-60); and the like. In some embodiments, portions or all of the minimal human synapsin 1 promoter (SYN) are used. Kugler et al., (2003) Gene Ther., 10 (4): 337-47; Thiel et al, (1991) Proc. Natl. Acad. Sci. USA, 88 (8) 3431-5; Castle et al., (2016) Methods Mol. Biol., 1382:133-49; McLean et al., (2014) Neurosci. Lett., 576:73-78; Kugler et al., (2003) Virology, 311 (1): 89-95.

In some embodiments, a tissue- or cell-specific promoter is configured to provide higher expression of an operatively linked minigene and/or transgene in a neuronal cell or tissue relative to that in a non-neuronal cell. In some embodiments, the neuron specific promoter is configured to provide higher expression of an operatively linked minigene and/or transgene in a neuron relative to that in a non-neuronal cell. Examples of neuronal cells or tissue include those comprising neurons, as well as Schwann cells, glial cells, astrocytes, etc. Examples of non-neuronal cells include, but are not limited to, hepatic cells, cardiomyocytes, red blood cells, epithelial cells etc. Higher levels of expression of an operatively linked minigene and/or transgene may include an increase in the number of RNA transcripts produced from transcription of the minigene and/or transgene. In some embodiments, the number of RNA transcripts produced may be measured by PCR. In some other embodiments, the number of RNA transcripts produced may be measured by RT-PCR, e.g., qPCR. In some embodiments, the number of RNA transcripts produced may be measured by sequencing. In some embodiments, the number of RNA transcripts produced may be measured by single-molecule Fluorescence In-Situ Hybridization (FISH). In some embodiments, the number of RNA transcripts produced may be measured by Northern blot analysis. Higher levels of expression of an operatively linked minigene and/or transgene may alternatively or in addition include an increase in the amount of protein produced, when the minigene and/or transgene encodes a protein of interest. In some embodiments, the amount of protein produced may be measured by an enzyme-linked immunosorbent assay (ELISA). In some embodiments, the amount of protein produced may be measured by Western blot analysis. In some embodiments, the amount of protein produced may be measured by immunostaining. In some embodiments, the amount of protein produced may be measured by time-resolved Forster Resonance Energy Transfer (TR-FRET). In some embodiments, the amount of protein produced may be measured by immunohistochemistry (IHC). In some embodiments, the level of expression is measured by more than one of these or other methods.

### Poly A Signal Sequence

In various embodiments, the nucleic acids, vectors and other compositions disclosed herein may comprise one or more polyadenylation (PolyA) signal sequences. The polyadenylation signal sequences may comprise a central sequence (e.g., AAUAAA) flanked by auxiliary sequence elements. Without being bound by theory, the sequence may signal the end of the transcript and serve as the site where a homopolymeric A sequence is added on the 3′ end by polyadenylate polymerase.

Polyadenylation signal sequences known in the art are contemplated, including but not limiting to the SV40 polyA, the human growth hormone (HGH) polyA, the bovine growth hormone (BGH) polyA, the beta-globin polyA, the alpha-globin polyA, the ovalbumin polyA, the kappa-light chain polyA, and a synthetic polyA. PolyA signal sequences may be used in the nucleic acids and other compositions disclosed herein.

### Post-Transcriptional Regulatory Elements

In various embodiments, the nucleic acids, transgenes, and other compositions disclosed herein may comprise one or more post-transcriptional regulatory elements (PREs), e.g., those that can enhance or otherwise improve expression of the transgene. Without being bound by the theory, PREs may enhance expression by enabling stability and 3′ end formation of mRNA, and/or may facilitate the nucleocytoplasmic export of unspliced mRNAs. PREs may also comprise binding sites for RNA-binding proteins (RBPs) or microRNAs.

Exemplary PREs include but are not limited to a PRE from the Hepatitis B virus (HPRE), bat virus (BPRE), ground squirrel virus (GSPRE), arctic squirrel virus (ASPRE), duck virus (DPRE), chimpanzee virus (CPRE) wooly monkey virus (WMPRE) or woodchuck virus (WPRE). In some embodiments, the nucleic acid or transgene comprises a PRE. In certain embodiments, the PRE comprises the HPRE. In some embodiments, a synthetic PRE is used.

### Viral Vectors

Also disclosed herein are vectors comprising the nucleic acids (e.g., minigenes, transgenes, other nucleic acid components such as promoters, PREs and polyAs, and combinations thereof) discussed herein. In some embodiments, a vector may serve to deliver a transgene to a target cell and/or to increase expression of that transgene in a target cell. In various embodiments, the vector may be used to regulate expression of proteins, antibodies or functional binding fragments, enzymes, etc., and/or nucleic acids, e.g., shRNA, siRNA, gRNA for use in CRISPR, etc., through use in combination with a splice modulator.

For instance, a vector may comprise a transcription factor described herein that increases the expression of a gene of interest. The vector may serve to transfer genetic information to another cell. Vectors may be used for cloning, e.g., as cloning vectors or plasmids. Vectors may also be designed specifically for other purposes, such as cellular infection, e.g., in a human neuronal cell, to drive expression, e.g., therapeutic protein and/or RNA expression. In some embodiments, vectors comprising the nucleic acids disclosed herein are contemplated. The vectors may be a DNA vector, a circular vector, or a plasmid. In some embodiments, the vector is double stranded. In other embodiments the vector is single stranded.

In some embodiments, the vector is a viral vector. In some embodiments, the vector is a viral vector used to deliver transgene sequence(s) to neuronal cells or tissue. Examples of viruses used for vectors include but are not limited to retroviruses, adenoviruses, lentiviruses, adeno-associated viruses, and other hybrid viruses. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

Without being bound by theory, viral vectors disclosed herein may insert their genomes into the host cell that they infect, thus delivering its nucleic acid sequence to the host. The viral genome inserted may be episomal or may be integrated into the chromosomes of the host cell at a site that may be random or targeted. In an embodiment, the vector is a viral vector used to deliver transgene sequences to cells. Examples of viruses used for vectors include but are not limited to retroviruses, adenoviruses, lentiviruses, adeno-associated viruses, and other hybrid viruses. Warnock et al., (2011) Methods Mol. Biol., 737:1-25. Lentivirus is a genus of retroviruses that can integrate significant amounts of viral DNA into a host cell, making them an efficient method of gene delivery. On the other hand, adenoviruses introduce genetic material that is not integrate into the chromosome of the host cell, thus reducing the risk of disrupting the host cell. In some embodiments, the viral vector is an adeno-associated viral (AAV) vector, chimeric AAV vector, adenoviral vector, retroviral vector, lentiviral vector, DNA viral vector, herpes simplex viral vector, baculoviral vector, or any mutant or derivative thereof.

In some embodiments, the vector comprising the transgene is or is derived from an adeno-associated virus (AAV). In some embodiments, the vector is a recombinant adeno-associated viral vector (rAAV). The rAAV genomes may comprise one or more AAV ITRs flanking a sequence encoding a transcription factor. In embodiments, the vectors additionaly comprise other transcriptional control elements such as those disclosed herein, e.g., promoter, enhancer, PRE, and/or polyA sequences that are functional in target cells to drive expression of the transgene sequence. The transgene sequence may also include intron sequences to facilitate processing of an RNA transcript when expressed in mammalian cells.

In various embodiments, the AAV vector, e.g., the rAAV vector, is a self-complementary AAV vector (scAAV). As used herein, “self-complementary” means the coding region has been designed to form an intra-molecular double-stranded template, e.g., in one or more inverted terminal repeats (ITRs). Without being bound by theory, a rate-limiting step for AAV genome often involves the second-strand synthesis since the typical AAV genome is a single-stranded DNA template. Ferrari et al, (1996) J. Virology, 70 (5): 3227-34; Fisher et al, (1996) J. Virology, 70 (1): 520-32. However, for scAAV genomes, upon infection, the two complementary halves of scAAV may associate to form one double stranded DNA (dsDNA) unit that is ready for replication and transcription rather than waiting for cell mediated synthesis of the second strand. In some embodiments, the rAAV vector disclosed herein is a scAAV vector and provides for faster and/or increased expression.

In some embodiments, the rAAV vectors disclosed herein lack one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVrh10, AAV.Anc80, AAV.Anc80L65, AAV-DJ, and AAV-DJ/8, AAVrh37, AAV-DJ, AAV-DJ/8, AAV-PHP.B, AAV-PHP.B2, AAV-PHP.B3, AAV-PHP.A, AAV-PHP.eB, and AAV-PHP.S. For instance, an AAV vector, e.g., an scAAV vector, may comprise nucleic acid sequences from an AAV2, e.g., ITR sequences from an AAV2. An AAV vector, e.g., an scAAV vector, may also comprise nucleic acids from more than one serotype. The nucleotide sequences of the genomes of the AAV serotypes are known in the art. For example, the complete genome of AAV1 is provided in GenBank Accession No. NC_002077; the complete genome of AAV2 is provided in GenBank Accession No. NC 001401 and Srivastava et al., Virol., 45:555-564 {1983): the complete genome of AAV3 is provided in GenBank Accession No. NC_1829; the complete genome of AAV4 is provided in GenBank Accession No. NC_001829; the AAV5 genome is provided in GenBank Accession No. AF085716; the complete genome of AAV-6 is provided in GenBank Accession No. NC_00 1862; at least portions of AAV7 and AAV8 genomes are provided in GenBank Accession Nos. AX753246 and AX753249, respectively; the AAV9 genome is provided in Gao et al., J. Virol., 78:6381-6388 (2004); the AAV10 genome is provided in Williams, (2006) Mol. Ther., 13 (1): 67-76; and the AAV11 genome is provided in Mori et al., (2004) Virology, 330 (2): 375-383.

In some embodiments, functional inverted terminal repeat (ITR) sequences may be used to support, e.g., the rescue, replication and packaging of the AAV virion. Thus, an AAV vector disclosed herein may include sequences that in cis provide for replication and packaging (e.g., functional ITRs) of the virus. The ITRs can be but need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, and AAV-11. The nucleotide sequences of the genomes of the AAV serotypes are known in the art. For example, the complete genome of AAV-1 is provided in GenBank Accession No. NC_002077; the complete genome of AAV-2 is provided in GenBank Accession No. NC 001401 and Srivastava et al., Virol., 45:555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., (2004) J. Virol., 78:6381-6388; the AAV-10 genome is provided in Williams, (2006) Mol. Ther., 13 (1): 67-76; and the AAV-11 genome is provided in Mori et al., (2004) Virology, 330 (2): 375-383. In one embodiment, the vector is an AAV-9 vector, with AAV-2 derived ITRs.

In some embodiments, the rAAV vector disclosed herein comprise one or more ITRs, e.g., two ITRs, with one upstream and the other downstream of a transgene (e.g., encoding hPGRN) and/or the other nucleic acid elements discussed above. In some embodiments, a nucleic acid disclosed herein, e.g., in an scAAV vector, comprises a first ITR that is disposed 5′ and a second ITR that is disposed 3′ to the promoter, minigene, transgene, post-transcriptional regulatory element, and/or polyA, e.g., wherein the ITRs are independently 1, 2, 3, 4, 5, 6, 7, 8, 9, 10, 11, 12, 13, 14, 15, 16, 17, 18, 19, 20, 50, 100, 150, 200, 250 nucleotides 5′ and/or 3′ of the other elements. An ITR sequence may be wild-type, or it may comprise one or more mutations, e.g., as long as it retains one or more function of a wild-type ITR. In some embodiments, wild-type ITR may be modified to comprise a deletion of a terminal resolution site. In some embodiments, an scAAV as disclosed herein may comprise two ITR sequences, where both are wild-type, variant, or modified AAV ITR sequences. In some embodiments, at least one ITR sequence is a wild-type, variant or modified AAV ITR sequence. In some embodiments, the two ITR sequences are both wild-type, variant or modified AAV ITR sequences. In some embodiments, the “left” or 5′-ITR is a modified AAV ITR sequence that allows for production of self-complementary genomes, and the “right” or 3′-ITR is a wild-type AAV ITR sequence. In some embodiments, the “right” or 3′-ITR is a modified AAV ITR sequence that allows for the production of self-complementary genomes, and the “left” or 5′-ITR is a wild-type AAV ITR sequence. In some embodiments, the ITR sequences are wild-type, variant, or modified AAV2 ITR sequences. In some embodiments, at least one ITR sequence is a wild-type, variant or modified AAV2 ITR sequence. In some embodiments, the two ITR sequences are both wild-type, variant or modified AAV2 ITR sequences. In some embodiments, the “left” or 5′-ITR is a modified AAV2 ITR sequence that allows for production of self-complementary genomes, and the “right” or 3′-ITR is a wild-type AAV2 ITR sequence. In some embodiments, the “right” or 3′-ITR is a modified AAV2 ITR sequence that allows for the production of self-complementary genomes, and the “left” or 5′-ITR is a wild-type AAV2 ITR sequence. Exemplary sequences that may be used for one or more of the ITRs are described herein. Embodiments of AAV ITRs provided in WO/2019/094253 (PCT/US2018/058744), which is incorporated herein by reference in its entirety, may also be used for any AAV ITR disclosed herein.

In some embodiments, the vector is an rAAV. In some embodiments, the rAAV vector lacks one or more (e.g., all) AAV rep and/or cap genes. An AAV vector may comprise (e.g., in its ITRs) nucleic acid sequences (e.g., DNA) from any suitable AAV serotype. Suitable AAV serotypes include, but are not limited to, AAV serotypes AAV-1, AAV-2, AAV-3, AAV-4, AAV-5, AAV-6, AAV-7, AAV-8, AAV-9, AAV-10 and AAV-11. For instance, an AAV vector, e.g., an scAAV vector, may comprise nucleic acid sequences from an AAV-2, e.g., ITR sequences from an AAV-2. An AAV vector, e.g., an scAAV vector, may also comprise nucleic acids from more than one serotype. GenBank Accession No. NC 001401 and Srivastava et al., Virol., 45:555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249; Gao et al., J. Virol., 78:6381-6388 (2004); Williams, (2006) Mol. Ther., 13 (1): 67-76; and Mori et al., (2004) Virology, 330 (2): 375-383.

In some embodiments, functional inverted terminal repeat (ITR) sequences in a viral vector may be used to support, e.g., the rescue, replication and packaging of the AAV virion. Thus, an AAV vector disclosed herein may include sequences that in cis provide for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging. The ITRs may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10 and AAV-11. GenBank Accession No. NC_002077; GenBank Accession No. NC 001401 and Srivastava et al., Virol., 45:555-564 {1983); GenBank Accession No. NC_1829; GenBank Accession No. NC_001829; GenBank Accession No. AF085716; GenBank Accession No. NC_00 1862; GenBank Accession Nos. AX753246 and AX753249, respectively; Gao et al., (2004) J. Virol., 78:6381-6388; Williams, (2006) Mol. Ther., 13 (1): 67-76; and Mori et al., (2004) Virology, 330 (2): 375-383. In one embodiment, the vector is an AAV-9 vector, with AAV-2 derived ITRs.

In some embodiments, a vector or nucleic acid sequence disclosed herein forms a cloning vector or an expression vector. In such embodiments, the vector may comprise other components that facilitate replication or maintenance of the vector. In some embodiments, the vector further comprises a selectable marker for clonal selection. In some embodiments, the selectable marker in the vector comprises a prokaryotic or eukaryotic antibiotic resistance gene. In some embodiments, the selectable marker in the vector comprises a kanamycin resistance gene. In some embodiments, the selectable marker in the vector comprises an ampicillin resistance gene. In some embodiments, the vector further comprises a puromycin resistance gene. In some embodiments, the selectable marker in the vector comprises a hygromycin resistance gene.

### Recombinant Virus

In various embodiments, the nucleic acids and vectors discussed herein may be present in one or more virus particle, such as a recombinant virus particle. Recombinant viruses are viruses generated by recombinant means. Various different viral types may be used, e.g., retroviruses, adenovirus, lentivirus, AAV, murine leukemia viruses, etc. Without being bound by theory, vectors delivered from retroviruses such as the lentivirus may provide for long-term gene transfer since they allow long-term, stable integration of a transgene and its propagation in daughter cells and may also provide low immunogenicity. Other suitable retroviruses include gammaretroviruses. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3 (6): 677-713. In some embodiments, the virus is a recombinant adenovirus comprising a nucleic acid or vector disclosed herein. In some embodiments, the virus is a recombinant AAV comprising a nucleic acid or vector disclosed herein.

In some embodiments, the nucleic acids or vectors disclosed herein are for use in the manufacture of a recombinant virus. In some embodiments, the nucleic acids or vectors disclosed herein are for use in the manufacture of an rAAV. Thus, also disclosed herein, in various embodiments, are virus compositions (also referred to as virions), e.g., rAAV virus compositions comprising a viral vector or nucleic acid disclosed above. In some embodiments, the recombinant virus is an adeno-associated virus (AAV) or any mutant or derivative thereof. In some embodiments, the recombinant virus is a chimeric AAV or any mutant or derivative thereof. In some embodiments, the recombinant virus is an adenovirus or any mutant or derivative thereof. In some embodiments, the recombinant virus is a retrovirus or any mutant or derivative thereof. In some embodiments, the recombinant virus is a lentivirus or any mutant or derivative thereof. In some embodiments, the recombinant virus is a DNA virus or any mutant or derivative thereof. In some embodiments, the recombinant virus is a herpes simplex virus or any mutant or derivative thereof. In some embodiments, the recombinant virus is a baculovirus or any mutant or derivative thereof.

In some embodiments, an AAV disclosed herein may comprise one or more AAV capsid proteins. AAV capsid proteins may be from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAVrh8, AAVfh10, AAV-DJ, AAV-DJ/8, AAV-PHP.B, AAV-PHP.B2, AAV-PHP.B3, AAV-PHP.A, AAV-PHP.eB, and AAV-PHP.S. In some embodiments, one or more capsid protein in an AAV is from an AAV-9. Without being bound by theory, typically in AAV, three capsid proteins, VP1, VP2 and VP3 multimerize to form the capsid. The polypeptide sequences of capsid proteins are known in the art, and can also be derived from the genome of the AAV. These can be used as exemplary capsids in the AAV virus compositions disclosed herein. For example, the complete genome of AAV-1 is provided in GenBank Accession No. NC_002077; the complete genome of AAV-2 is provided in GenBank Accession No. NC 001401 and Srivastava et al., Virol., 45:555-564 {1983): the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78:6381-6388 (2004); the AAV-10 genome is provided in Williams, (2006) Mol. Ther., 13 (1): 67-76; and the AAV-11 genome is provided in Mori et al., (2004) Virology, 330 (2): 375-383. Capsid proteins AAV-PHP.B, AAV-PHP.B2, AAV-PHP.B3, AAV-PHP.A, AAV-PHP.eB, or AAV-PHP.S are provided in Deverman et al., (2016) Nat. Biotech., 34:204-209 and Chan et al., (2017) Nat. Neurosci., 20:1172-1179. In some embodiments, the recombinant virus is an AAV comprising one or more AAV1, AAV2, AAV3, AAV4, AAV5, AAV6, AAV7, AAV 8, AAV9, AAV10, and AAV11, AAV 12, AAVrh8, AAVrh10, AAV-DJ, AAV-DJ/8, AAV-PHP.B, AAV-PHP.B2, AAV-PHP. B3, AAV-PHP.A, AAV-PHP.eB, or AAV-PHP.S capsid serotype, or a functional variant thereof. In some embodiments, the recombinant virus is an AAV comprising a combination of capsids from more than one AAV serotype.

In some embodiments, AAV compositions disclosed herein comprise one or more cis-acting sequences directing viral DNA replication (rep), encapsidation/packaging and host cell chromosome integration are contained within the ITRs. In some embodiments, one or more of these sequences may also be present in trans rather than cis, e.g., on a separate plasmid during the virus manufacturing process in a host cell. Typically, three AAV promoters (named p5, p19, and p40 for their relative map locations) drive the expression of the two AAV internal open reading frames encoding rep and cap genes in wild-type virus. In some embodiments, one or more of these promoters and/or open reading frames are present in cis in an AAV vector and/or AAV virion disclosed herein, or are present on separate plasmids during the AAV virus manufacturing process, e.g., in a host cell producing the virus. The two rep promoters (p5 and p19), coupled with the differential splicing of the single AAV intron (at nucleotides 2107 and 2227), may result in the production of four rep proteins (rep 78, rep 68, rep 52, and rep 40) from the rep gene. Rep proteins possess multiple enzymatic properties that are ultimately responsible for replicating the viral genome. The cap gene is typically expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, (1992) Curr. Topics Microbiol. Imm., 158:97-129.

In some embodiments, the AAV capsid proteins VP1, VP2, VP3 used in the AAV disclosed herein are encoded by or comprise the following sequences:

VP1 nucleic acid (SEQ ID NO: 84):
atggctgccgatggttatcttccagattggctcgaggacaaccttagtgaaggaattcgcgagtggt
gggctttgaaacctggagcccctcaacccaaggcaaatcaacaacatcaagacaacgctcgaggtcttgtgcttccg
ggttacaaataccttggacccggcaacggactcgacaagggggagccggtcaacgcagcagacgcggcggccct
cgagcacgacaaggcctacgaccagcagctcaaggccggagacaacccgtacctcaagtacaaccacgccgac
gccgagttccaggagcggctcaaagaagatacgtcttttgggggcaacctcgggcgagcagtcttccaggccaaaa
agaggcttcttgaacctcttggtctggttgaggaagcggctaagacggctcctggaaagaagaggcctgtagagcag
tctcctcaggaaccggactcctccgcgggtattggcaaatcgggtgcacagcccgctaaaaagagactcaatttcggt
cagactggcgacacagagtcagtcccagaccctcaaccaatcggagaacctcccgcagccccctcaggtgtggga
tctcttacaatggcttcaggtggtggcgcaccagtggcagacaataacgaaggtgccgatggagtgggtagttcctcg
ggaaattggcattgcgattcccaatggctgggggacagagtcatcaccaccagcacccgaacctgggccctgccca
cctacaacaatcacctctacaagcaaatctccaacagcacatctggaggatcttcaaatgacaacgcctacttcggct
acagcaccccctgggggtattttgacttcaacagattccactgccacttctcaccacgtgactggcagcgactcatcaa
caacaactggggattccggcctaagcgactcaacttcaagctcttcaacattcaggtcaaagaggttacggacaaca
atggagtcaagaccatcgccaataaccttaccagcacggtccaggtcttcacggactcagactatcagctcccgtacg
tgctcgggtcggctcacgagggctgcctcccgccgttcccagcggacgttttcatgattcctcagtacgggtatctgacg
cttaatgatggaagccaggccgtgggtcgttcgtccttttactgcctggaatatttcccgtcgcaaatgctaagaacgggt
aacaacttccagttcagctacgagtttgagaacgtacctttccatagcagctacgctcacagccaaagcctggaccga
ctaatgaatccactcatcgaccaatacttgtactatctctcaaagactattaacggttctggacagaatcaacaaacgct
aaaattcagtgtggccggacccagcaacatggctgtccagggaagaaactacatacctggacccagctaccgaca
acaacgtgtctcaaccactgtgactcaaaacaacaacagcgaatttgcttggcctggagcttcttcttgggctctcaatg
gacgtaatagcttgatgaatcctggacctgctatggccagccacaaagaaggagaggaccgtttctttcctttgtctgga
tctttaatttttggcaaacaaggaactggaagagacaacgtggatgcggacaaagtcatgataaccaacgaagaag
aaattaaaactactaacccggtagcaacggagtcctatggacaagtggccacaaaccaccagagtgcccaagcac
aggcgcagaccggctgggttcaaaaccaaggaatacttccgggtatggtttggcaggacagagatgtgtacctgcaa
ggacccatttgggccaaaattcctcacacggacggcaactttcacccttctccgctgatgggagggtttggaatgaagc
acccgcctcctcagatcctcatcaaaaacacacctgtacctgcggatcctccaacggccttcaacaaggacaagctg
aactctttcatcacccagtattctactggccaagtcagcgtggagatcgagtgggagctgcagaaggaaaacagcaa
gcgctggaacccggagatccagtacacttccaactattacaagtctaataatgttgaatttgctgttaatactgaaggtgt
atatagtgaaccccgccccattggcaccagatacctgactcgtaatctgtaa
VP2 nucleic acid (SEQ ID NO: 85):
acggctcctggaaagaagaggcctgtagagcagtctcctcaggaaccggactcctccgcgggta
ttggcaaatcgggtgcacagcccgctaaaaagagactcaatttcggtcagactggcgacacagagtcagtcccaga
ccctcaaccaatcggagaacctcccgcagccccctcaggtgtgggatctcttacaatggcttcaggtggtggcgcacc
agtggcagacaataacgaaggtgccgatggagtgggtagttcctcgggaaattggcattgcgattcccaatggctgg
gggacagagtcatcaccaccagcacccgaacctgggccctgcccacctacaacaatcacctctacaagcaaatctc
caacagcacatctggaggatcttcaaatgacaacgcctacttcggctacagcaccccctgggggtattttgacttcaac
agattccactgccacttctcaccacgtgactggcagcgactcatcaacaacaactggggattccggcctaagcgactc
aacttcaagctcttcaacattcaggtcaaagaggttacggacaacaatggagtcaagaccatcgccaataaccttacc
agcacggtccaggtcttcacggactcagactatcagctcccgtacgtgctcgggtcggctcacgagggctgcctcccg
ccgttcccagcggacgttttcatgattcctcagtacgggtatctgacgcttaatgatggaagccaggccgtgggtcgttcg
tccttttactgcctggaatatttcccgtcgcaaatgctaagaacgggtaacaacttccagttcagctacgagtttgagaac
gtacctttccatagcagctacgctcacagccaaagcctggaccgactaatgaatccactcatcgaccaatacttgtact
atctctcaaagactattaacggttctggacagaatcaacaaacgctaaaattcagtgtggccggacccagcaacatg
gctgtccagggaagaaactacatacctggacccagctaccgacaacaacgtgtctcaaccactgtgactcaaaaca
acaacagcgaatttgcttggcctggagcttcttcttgggctctcaatggacgtaatagcttgatgaatcctggacctgctat
ggccagccacaaagaaggagaggaccgtttctttcctttgtctggatctttaatttttggcaaacaaggaactggaaga
gacaacgtggatgcggacaaagtcatgataaccaacgaagaagaaattaaaactactaacccggtagcaacgga
gtcctatggacaagtggccacaaaccaccagagtgcccaagcacaggcgcagaccggctgggttcaaaaccaag
gaatacttccgggtatggtttggcaggacagagatgtgtacctgcaaggacccatttgggccaaaattcctcacacgg
acggcaactttcacccttctccgctgatgggagggtttggaatgaagcacccgcctcctcagatcctcatcaaaaacac
acctgtacctgcggatcctccaacggccttcaacaaggacaagctgaactctttcatcacccagtattctactggccaa
gtcagcgtggagatcgagtgggagctgcagaaggaaaacagcaagcgctggaacccggagatccagtacacttc
caactattacaagtctaataatgttgaatttgctgttaatactgaaggtgtatatagtgaaccccgccccattggcaccag
atacctgactcgtaatctgtaa
VP3 nucleic acid (SEQ ID NO: 86):
atggcttcaggtggtggcgcaccagtggcagacaataacgaaggtgccgatggagtgggtagtt
cctcgggaaattggcattgcgattcccaatggctgggggacagagtcatcaccaccagcacccgaacctgggccct
gcccacctacaacaatcacctctacaagcaaatctccaacagcacatctggaggatcttcaaatgacaacgcctactt
cggctacagcaccccctgggggtattttgacttcaacagattccactgccacttctcaccacgtgactggcagcgactc
atcaacaacaactggggattccggcctaagcgactcaacttcaagctcttcaacattcaggtcaaagaggttacggac
aacaatggagtcaagaccatcgccaataaccttaccagcacggtccaggtcttcacggactcagactatcagctccc
gtacgtgctcgggtcggctcacgagggctgcctcccgccgttcccagcggacgttttcatgattcctcagtacgggtatct
gacgcttaatgatggaagccaggccgtgggtcgttcgtccttttactgcctggaatatttcccgtcgcaaatgctaagaa
cgggtaacaacttccagttcagctacgagtttgagaacgtacctttccatagcagctacgctcacagccaaagcctgg
accgactaatgaatccactcatcgaccaatacttgtactatctctcaaagactattaacggttctggacagaatcaacaa
acgctaaaattcagtgtggccggacccagcaacatggctgtccagggaagaaactacatacctggacccagctacc
gacaacaacgtgtctcaaccactgtgactcaaaacaacaacagcgaatttgcttggcctggagcttcttcttgggctctc
aatggacgtaatagcttgatgaatcctggacctgctatggccagccacaaagaaggagaggaccgtttctttcctttgtc
tggatctttaatttttggcaaacaaggaactggaagagacaacgtggatgcggacaaagtcatgataaccaacgaag
aagaaattaaaactactaacccggtagcaacggagtcctatggacaagtggccacaaaccaccagagtgcccaa
gcacaggcgcagaccggctgggttcaaaaccaaggaatacttccgggtatggtttggcaggacagagatgtgtacct
gcaaggacccatttgggccaaaattcctcacacggacggcaactttcacccttctccgctgatgggagggtttggaatg
aagcacccgcctcctcagatcctcatcaaaaacacacctgtacctgcggatcctccaacggccttcaacaaggacaa
gctgaactctttcatcacccagtattctactggccaagtcagcgtggagatcgagtgggagctgcagaaggaaaaca
gcaagcgctggaacccggagatccagtacacttccaactattacaagtctaataatgttgaatttgctgttaatactgaa
ggtgtatatagtgaaccccgccccattggcaccagatacctgactcgtaatctgtaa
VP1 Protein (SEQ ID NO: 87):
MAADGYLPDWLEDNLSEGIREWWALKPGAPQPKANQQHQDNARGL
VLPGYKYLGPGNGLDKGEPVNAADAAALEHDKAYDQQLKAGDNPYLKYNHADAEF
QERLKEDTSFGGNLGRAVFQAKKRLLEPLGLVEEAAKTAPGKKRPVEQSPQEPDS
SAGIGKSGAQPAKKRLNFGQTGDTESVPDPQPIGEPPAAPSGVGSLTMASGGGAP
VADNNEGADGVGSSSGNWHCDSQWLGDRVITTSTRTWALPTYNNHLYKQISNSTS
GGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQ
YGYLTLNDGSQAVGRSSFYCLEYFPSQMLRTGNNFQFSYEFENVPFHSSYAHSQS
LDRLMNPLIDQYLYYLSKTINGSGQNQQTLKFSVAGPSNMAVQGRNYIPGPSYRQQ
RVSTTVTQNNNSEFAWPGASSWALNGRNSLMNPGPAMASHKEGEDRFFPLSGSLI
FGKQGTGRDNVDADKVMITNEEEIKTTNPVATESYGQVATNHQSAQAQAQTGWVQ
NQGILPGMVWQDRDVYLQGPIWAKIPHTDGNFHPSPLMGGFGMKHPPPQILIKNTP
VPADPPTAFNKDKLNSFITQYSTGQVSVEIEWELQKENSKRWNPEIQYTSNYYKSN
NVEFAVNTEGVYSEPRPIGTRYLTRNL
VP2 Protein (SEQ ID NO: 88):
TAPGKKRPVEQSPQEPDSSAGIGKSGAQPAKKRLNFGQTGDTESVP
DPQPIGEPPAAPSGVGSLTMASGGGAPVADNNEGADGVGSSSGNWHCDSQWLG
DRVITTSTRTWALPTYNNHLYKQISNSTSGGSSNDNAYFGYSTPWGYFDFNRFHCH
FSPRDWQRLINNNWGFRPKRLNFKLFNIQVKEVTDNNGVKTIANNLTSTVQVFTDS
DYQLPYVLGSAHEGCLPPFPADVFMIPQYGYLTLNDGSQAVGRSSFYCLEYFPSQM
LRTGNNFQFSYEFENVPFHSSYAHSQSLDRLMNPLIDQYLYYLSKTINGSGQNQQT
LKFSVAGPSNMAVQGRNYIPGPSYRQQRVSTTVTQNNNSEFAWPGASSWALNGR
NSLMNPGPAMASHKEGEDRFFPLSGSLIFGKQGTGRDNVDADKVMITNEEEIKTTN
PVATESYGQVATNHQSAQAQAQTGWVQNQGILPGMVWQDRDVYLQGPIWAKIPH
TDGNFHPSPLMGGFGMKHPPPQILIKNTPVPADPPTAFNKDKLNSFITQYSTGQVSV
EIEWELQKENSKRWNPEIQYTSNYYKSNNVEFAVNTEGVYSEPRPIGTRYLTRNL
VP3 Protein (SEQ ID NO: 89):
MASGGGAPVADNNEGADGVGSSSGNWHCDSQWLGDRVITTSTRT
WALPTYNNHLYKQISNSTSGGSSNDNAYFGYSTPWGYFDFNRFHCHFSPRDWQR
LINNNWGFRPKRLNFKLFNIQVKEVTDNNGVKTIANNLTSTVQVFTDSDYQLPYVLG
SAHEGCLPPFPADVFMIPQYGYLTLNDGSQAVGRSSFYCLEYFPSQMLRTGNNFQ
FSYEFENVPFHSSYAHSQSLDRLMNPLIDQYLYYLSKTINGSGQNQQTLKFSVAGPS
NMAVQGRNYIPGPSYRQQRVSTTVTQNNNSEFAWPGASSWALNGRNSLMNPGPA
MASHKEGEDRFFPLSGSLIFGKQGTGRDNVDADKVMITNEEEIKTTNPVATESYGQ
VATNHQSAQAQAQTGWVQNQGILPGMVWQDRDVYLQGPIWAKIPHTDGNFHPSP
LMGGFGMKHPPPQILIKNTPVPADPPTAFNKDKLNSFITQYSTGQVSVEIEWELQKE
NSKRWNPEIQYTSNYYKSNNVEFAVNTEGVYSEPRPIGTRYLTRNL

In one embodiment, the recombinant virus is an AAV comprising an AAV9 capsid serotype or any mutant or derivative thereof. In some embodiments, the recombinant virus comprises AAV9 capsid proteins VP1, VP2 and VP3. In some embodiments, the recombinant virus is a scAAV.

In various embodiments, the target cells of this disclosure may be any mammalian cell type. In some aspects of this disclosure, the nucleic acids and vectors regulate expression in a neuronal tissue or fluid or cell. In some embodiments, the neuronal tissue is the brain. In some embodiments, the neuronal tissue is the frontal lobe of the brain. In some embodiments, the neuronal tissue is the temporal lobe of the brain. In some embodiments, the neuronal tissue is the central nervous system. In some embodiments, the neuronal tissue is the spinal cord. In some embodiments, the neuronal cell is a human neuronal cell. In some embodiments, the neuronal cell is a neuron. In some embodiments, the neuronal cell is an astrocyte. In some embodiments, the neuronal fluid is cerebrospinal fluid. In some embodiments, a non-neuronal tissue is the liver. In some embodiments, the non-neuronal fluid is plasma. In some embodiments, a non-neuronal cell is a hepatocyte. In some embodiments, a non-neuronal cell is a stellate fat storing cell. In some embodiments, a non-neuronal cell is a Kupffer cell. In some embodiments, a non-neuronal cell is a liver endothelial cell. In some embodiments, the non-neuronal fluid is plasma. In some embodiments, the non-neuronal fluid is serum. In some embodiments, the non-neuronal fluid is blood.

### Methods of Producing Recombinant Virus

Also disclosed herein, in various embodiments, are methods of producing recombinant virus comprising neuron specific promoters. In some embodiments, nucleic acid sequences, e.g., plasmids encoding an AAV or other viral genome, are used to produce the recombinant virus. In some embodiments, nucleic acid sequences, e.g., plasmids, comprising an AAV rep gene and/or an AAV cap gene are also used in preparing the AAV or other virus. Also disclosed herein are nucleic acid sequences, e.g., plasmids, comprising an adenovirus helper function gene. In some embodiments, the nucleic acids encoding the AAV rep, AAV cap, and/or adenovirus helper genes may be present in the same structure, e.g., a single plasmid, or they may be present in separate structures. In some embodiments, the one or more plasmids are cotransfected with the nucleic acid encoding the AAV vector into competent cells, and the cells are cultured to produce the recombinant virus. In some cases, the plasmids encoding AAV viral genome and AAV rep and/or cap genes are transferred to cells permissible for infection with a helper virus of AAV (e.g., adenovirus, E1-deleted adenovirus or herpesvirus). In some embodiments, the rAAV genome is assembled into infectious viral particles with AAV capsid proteins in the cells after transfection. Techniques to produce rAAV particles, in which an AAV genome to be packaged, rep and cap genes, and helper virus functions are provided to a cell are known in the art and may include, e.g., electroporation. In some embodiments, production of rAAV involves the following components present within a single cell (denoted herein as a packaging cell): a rAAV vector, AAV rep and cap genes separate from (i.e., not in) the rAAV vector, and helper virus functions. Production of pseudotyped rAAV is disclosed in, for example, WO 01/83692 which is incorporated by reference herein in its entirety. In various embodiments, AAV capsid proteins may be modified to enhance delivery of the recombinant vector. Modifications to capsid proteins are generally known in the art. See, for example, US 2005/0053922 and US 2009/0202490, the disclosures of which are incorporated by reference herein in their entirety.

In various embodiments, general principles of viral vector production may be utilized to produce the vectors and virus, e.g., rAAV, disclosed herein. Carter, (1992) Curr. Opinions Biotech., 1533-539; Muzyczka, (1992) Curr. Topics Microbial. Immunol., 158:97-129. Various approaches are disclosed in Ratschin et al., (1984) Mol. Cell. Biol., 4:2072; Hennonat et al., (1984) Proc. Natl. Acad. Sci. USA, 81:6466; Tratschin et al., (1985) Mol. Cell. Biol., 5:3251; Mclaughlin et al., (1988) J. Virol., 62:1963; Lebkowski et al., (1988) Mol. Cell. Biol., 7:349; Samulski et al. (1989) J. Virol., 63:3822-3828; U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., (1995) Vaccine, 13:1244-1250; Paul et al., (1993) Hum. Gene Ther., 4:609-615; Clark et al. (1996) Gene Therapy, 3:1124-1132; U.S. Pat. Nos. 5,786,211; 5,871,982; and 6,258,595. The foregoing documents are hereby incorporated by reference in their entirety herein, with particular emphasis on those sections of the documents relating to rAAV production.

An exemplary method of generating a packaging cell is to create a cell line that stably expresses all the necessary components for AAV particle production. For example, a plasmid (or multiple plasmids) encoding a rAAV vector lacking AAV rep and cap genes, AAV rep and cap genes separate from the rAAV vector, and a selectable marker, such as a neomycin resistance gene, are integrated into the genome of a cell. AAV genomes have been introduced into bacterial plasmids by procedures such as GC tailing (Samulski et al., (1982) Proc. Natl. Acad. Sci. USA, 79:2077-2081), addition of synthetic linkers containing restriction endonuclease cleavage sites (Laughlin et al., (1983) Gene, 23:65-73) or by direct, blunt-end ligation (Senapathy et al., (1984) J. Biol. Chem., 259:4661-4666). The packaging cell line is then infected with a helper virus such as adenovirus and/or a plasmid encoding a helper virus. The advantages of this method are that the cells are selectable and are suitable for large-scale production of rAAV. Other examples of suitable methods employ adenovirus or baculovirus rather than plasmids to introduce rAAV vectors and/or rep and cap genes into packaging cells.

In some embodiments, a method of producing recombinant virus comprises providing a nucleic acid to be packaged. In some embodiments, the nucleic acid is a plasmid. In other embodiments, the nucleic acid comprises a transgene sequence interposed between a first AAV terminal repeat and a second AAV terminal repeat. In some embodiments, the transgene encodes human progranulin (hPGRN). In some embodiments, the method of producing recombinant virus comprises providing one or more additional nucleic acids. In some embodiments, the one or more additional nucleic acids comprises an AAV rep gene and/or an AAV cap gene. In some embodiments, the one or more additional nucleic acids comprises an AAV rep gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises an AAV cap gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises one or more of an adenovirus helper function gene.

In some embodiments, the nucleic acids are co-transfected into competent cells or packaging cells. Methods of co-transfection are known in the art, and include, but are not limited to, transfection by lipofectamine, electroporation, and polyethylenimine. Competent cells or packaging cells may be non-adherent cells cultured in suspension or adherent cells. In one embodiment any suitable packaging cell line may be used, such as Hela cells, HEK 293 cells and PerC.6 cells (a cognate 293 line). In one embodiment, the packaging cells are human cells. In one embodiment, the packaging cells are HEK 293 cells. In one embodiment, the packaging cells are insect cells. In one embodiment, the packaging cells are Sf9 cells. In some embodiments, the method comprises culturing the transfected cells to produce recombinant virus. In some embodiments, the method comprises recovering the recombinant virus. Methods of recovering recombinant virus include, e.g., those disclosed in U.S. Pat. Nos. 6,143,548 and 9,408,904. In some embodiments, recombinant virus is secreted into cell culture media and purified from the media. In some embodiments, packaging cells are lysed, and the contents purified to recover the recombinant virus. In some embodiments, the virus is recovered from the packaging cell by filtration or centrifugation. In some embodiments, the virus is recovered from the packaging cell by chromatography.

In various embodiments, disclosed herein are cells comprising the nucleic acids disclosed herein, cells comprising the vectors disclosed herein, or cells comprising the viruses disclosed herein. The cells comprising the nucleic acids disclosed herein, cells comprising the vectors disclosed herein, or cells comprising the viruses disclosed herein, may be human cells. The cells comprising the nucleic acids disclosed herein, cells comprising the vectors disclosed herein, or cells comprising the viruses disclosed herein, may also be insect cells. In some embodiments, the cells comprising the nucleic acids disclosed herein, cells comprising the vectors disclosed herein, or cells comprising the viruses disclosed herein are HEK293 cells. In some other embodiments, the cells comprising the nucleic acids disclosed herein, cells comprising the vectors disclosed herein, or cells comprising the viruses disclosed herein are Sf9 cells.

In some embodiments, the method of producing recombinant virus comprises transfecting an insect cell. In some embodiments, the method comprises transfecting an insect cell with a baculovirus comprising the nucleic acids as disclosed herein. In some embodiments, the method comprises transfecting an insect cell with baculovirus comprising a nucleic acid comprising a transgene sequence interposed between a first AAV terminal repeat and a second AAV terminal repeat. In some embodiments, the method comprises transfecting an insect cell with a baculovirus comprising one or more additional nucleic acids. In some embodiments, the one or more additional nucleic acids comprises an AAV rep gene and/or an AAV cap gene. In some embodiments, the one or more additional nucleic acids comprises an AAV rep gene derived from an AAV serotype 1, AAV serotype 2, AAV serotype 3, AAV serotype 4, AAV serotype 5, AAV serotype 6, AAV serotype 7, AAV serotype 8, or AAV serotype 9. In some embodiments, the one or more additional nucleic acids comprises an AAV cap gene derived from an AAV serotype 1, AAV serotype 2, AAV serotype 3, AAV serotype 4, AAV serotype 5, AAV serotype 6, AAV serotype 7, AAV serotype 8, or AAV serotype 9.c. In some embodiments, the one or more additional nucleic acids comprises one or more of an adenovirus helper function gene. In some embodiments, the insect cells are cultivated under conditions suitable to produce recombinant virus. In some embodiments, the virus is recovered from the insect cell. In some embodiments, the virus is recovered from the insect cell by filtration or centrifugation. In some embodiments, the virus is recovered from the insect cell by chromatography.

### Pharmaceutical Compositions

In various embodiments, pharmaceutical compositions are disclosed. In some embodiments, a pharmaceutical composition comprises one or more nucleic acids, vectors and/or viruses disclosed herein. In some embodiments, the pharmaceutical composition comprises a pharmaceutically acceptable carrier.

The nucleic acids, vectors, and/or recombinant virus according to the present disclosure (e.g., viral particles) can be formulated to prepare pharmaceutically useful compositions. Exemplary formulations include, for example, those disclosed in U.S. Pat. Nos. 9,051,542 and 6,703,237, which are incorporated by reference in their entirety. The compositions of the disclosure can be formulated for administration to a mammalian subject, e.g., a human. In some embodiments, delivery systems may be formulated for intramuscular, intradermal, mucosal, subcutaneous, intravenous, intrathecal, injectable depot type devices, or topical administration.

In some embodiments, when the delivery system is formulated as a solution or suspension, the delivery system is in an acceptable carrier, e.g., an aqueous carrier. A variety of aqueous carriers may be used, e.g., water, buffered water, 0.8% saline, 0.3% glycine, hyaluronic acid and the like. These compositions may be sterilized and/or sterile filtered. The resulting aqueous solutions may be packaged for use as is, or lyophilized. In some embodiments, the lyophilized preparation is combined with a sterile solution prior to administration.

In some embodiments, the compositions, e.g., pharmaceutical compositions, may contain pharmaceutically acceptable auxiliary substances to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. In some embodiments, the pharmaceutical composition comprises a preservative. In some other embodiments, the pharmaceutical composition does not comprise a preservative.

### Method of Use and Treatment

Without being bound by theory, the nucleic acids and other embodiments described herein are used in a method of conditionally expressing a molecule (e.g., gene of interest), said method comprising: administering an expression system, e.g. a cell comprising the nucleic acid molecule described herein, a vector described herein to a subject in need thereof, wherein: a) expression of said gene of interest is increased, e.g., 2, 3, 4, 5, 6, 7, 8, 9, 10, 20, 30, 50, or 100 fold greater, relative to the level of expression of said gene of interest in the subject without such treatment.

In one embodiment, the term “treating” comprises the step of administering an effective dose, or effective multiple doses, of a composition comprising a nucleic acid, a vector, a recombinant virus, or a pharmaceutical composition as disclosed herein, to an animal (including a human being) in need thereof. If the dose is administered prior to development of a disorder/disease, the administration is prophylactic. If the dose is administered after the development of a disorder/disease, the administration is therapeutic. In embodiments, an effective dose is a dose that detectably alleviates (either eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival. The term encompasses but does not require complete treatment (i.e., curing) and/or prevention. In some embodiments, an effective dose comprises 1×10¹⁰ to 1×10¹⁵ vector genome per milliliter (vg/ml) of a virus as disclosed herein. In some embodiments, an effective dose comprises 1×10⁶ to 1×10¹⁰ plaque forming units per milliliter (pfu/ml) of a virus as disclosed herein. In some embodiments, an effective dose comprises 1×10⁶ to 1×10⁹ transducing units per milliliter (TU/ml) of a virus as disclosed herein. Examples of disease states contemplated for treatment are set out herein.

In some embodiments, the disease being treated is caused by mutations in the gene of interest, In some embodiments, the mutations in the gene of interest are deletion mutations. In some embodiments, the mutations in the gene of interest are null mutations. In some embodiments, the mutations in the gene of interest are indels. In some embodiments, the mutations in the gene of interest are loss-of-function mutations. In some embodiments, the mutations in the gene of interest are knock-out mutations. In some embodiments, the mutations in the gene of interest results in loss of expression and/or function of the protein. In some embodiments, a patient in need of treatment with the nucleic acids, vectors, and/or viruses disclosed herein is identified by screening for mutation prior to administration. In some embodiments, screening comprises obtaining a sample of cells or tissue from a subject and sequencing or genotyping one or more genetic loci in the sample to check for the presence of a mutation. In some embodiments, the screening is performed on genetic material from samples such as (but not limited to) saliva, blood, and/or skin cells.

In some embodiments, a nucleic acid, vector, recombinant virus, or pharmaceutical compositions disclosed herein is used in the manufacture of a medicament, for treating a subject in need thereof. In embodiments, the subject suffers from a disorder caused by one or more mutations in the gene of interest.

In various embodiments, the nucleic acid, vector, recombinant virus, or pharmaceutical composition disclosed herein may be delivered to the subject in need thereof by an intravenous administration, direct brain administration (e.g., intrathecal, intracerebral, and/or intraventricular administration), intranasal administration, intra-aural administration, or intra-ocular route administration, or any combination thereof. In some embodiments, the nucleic acid, vector, recombinant virus, or pharmaceutical composition is delivered by intrathecal administration. In some embodiments, the nucleic acid, vector, recombinant virus, or pharmaceutical composition is delivered by an intracerebral or intraventricular route of administration. In some embodiments, the administered nucleic acid, vector, recombinant virus, or pharmaceutical composition is ultimately delivered to the brain, spinal cord, peripheral nervous system, and/or CNS, either directly or by transfer after administration to a separate tissue or fluid, e.g., blood.

Without being bound by theory, in some embodiments the methods disclosed herein may rescue cells that carry mutations on a gene coding for a polypeptide, that result in a non-functioning polypeptide. In some embodiments, a method of expressing a molecule, for example a protein or ribonucleic acid (e.g., an siRNA), comprises delivering to a cell a nucleic acid, viral vector, virus, or pharmaceutical composition disclosed herein. In some embodiments, the cell is a neuronal cell. In some embodiments, the cell is a mammalian cell. In some embodiments, the cell is a human cell. In some embodiments, the neuronal cell is a neuron. In some embodiments, delivery is done in vitro. In some embodiments, delivery is done ex vivo. In some embodiments, the delivery is by systemic administration. In some embodiments, the delivery is local. In some embodiments, the delivery is by direct application to the target tissue. In some embodiments, the target tissue is the brain. In some embodiments, the delivery is by injection into the brain. In some embodiments, the delivery is by intrathecal administration. Without being bound by theory, the methods disclosed herein may reduce lipofuscin deposition, astrocyte and microglia activation, and/or inflammation in the brain of a human or mouse with a mutation in the gene of interest, thus providing potential benefits to subjects in need thereof.

In various embodiments, the nucleic acids, vectors, viruses, and pharmaceutical compositions disclosed herein may be used to treat a disorder. In some embodiments, a nucleic acid, vector, viruse, and/or pharmaceutical composition disclosed herein may be used in the manufacture of a medicament for treating a disorder. In some embodiments, the disorder is caused by one or more mutations in the gene of interest.

Also provided herein is a kit comprising a nucleic acid molecule described herein, a vector described herein, a recombinant virus described herein, a cell described herein, or a pharmaceutical composition described herein.

The details of one or more embodiments of the disclosure are set forth in the accompanying description above. Although any methods and materials similar or equivalent to those described herein can be used in the practice or testing of the present disclosure, the preferred methods and materials are now described. Other features, objects, and advantages of the disclosure will be apparent from the description and from the claims. In the specification and the appended claims, the singular forms include plural referents unless the context clearly dictates otherwise. Unless defined otherwise, all technical and scientific terms used herein have the same meaning as commonly understood by one of ordinary skill in the art to which this disclosure belongs. All patents and publications cited in this specification are incorporated by reference as applicable, unless otherwise indicated. The following Examples are presented in order to more fully illustrate the preferred embodiments of the disclosure. These examples should in no way be construed as limiting the scope of the disclosed subject matter, which is defined by the appended claims.

### EXAMPLES

### Example 1: TRE Reporters

All the TRE reporter constructs were modified from pLVX-TetOne-Puro (TaKaRa) by removing hPGK promoter and Tet-On 3G coding region, inserting firefly luciferase coding sequence into the multi-cloning site down stream of TRE3Gs promoter, and then deleting multiple tetracycline response elements (TRE), reducing the repeats from 7 to 3, 2, and 1 to generate 3×TRE-, 2×TRE-, and 1×TRE-reporter, respectively.

### Example 2: Human and Mouse SCN1A Reporters

For human SCN1A promoter reporter, a fragment of 2673 base pairs (bps) promoter plus 242 bps exon sequence of human scn1a gene correlates to GRCh38/hg38 chr2: 166127806-166130720 (-strand) was PCR amplified from human genomic DNA and cloned into upstream of a firefly luciferase reporter gene. For mouse SCN1A promoter reporter, sequence including 3064 bps promoter and 256 bps exon sequence of mouse scn1a gene was amplified by PCR from genomic DNA of C57BL/6 mouse strain. The sequence correlates to mouse genome reference sequence GRCm38/mm10 chr2: 66409862-66413181 (-strand), and was cloned into a vector directly upstream of a firefly luciferase reporter gene.

### Example 3: Normalizing Reporter Construct

Renilla luciferase gene was cloned directly downstream of the hGPK promoter in a separate construct. Renilla luciferase was used to normalize firefly luciferase activity in all transient transfection reporter assays.

### Example 4: Cas9 Activator Constructs

A mini nuclease dead Staphylococcus aureus Cas9 (mini Sa-dCas9) (Ma et al. 2018) was synthesized and cloned into a mammalian expression vector directly downstream of the elongation factor 1α promoter (EF1a) promoter. In some cases, it was cloned into a vector and driven by the mammalian ubiquitin C (UbC) promoter. Transcription activation domains (TADs) including VP64, p65 and Rta (VPR) and VPR3 (Chavez et al. 2015, Ma et al. 2018) coding sequences were linked to the 3′-end of Cas9 gene. Human influenza hemagglutinin (HA) tag sequence was in-frame linked to the TAD sequence. Three nuclear localization sequences (NLS) were placed at N-terminal end of Sa-dCas9, between Cas9 and TADs, and between TADs and HA tag.

### Example 5: sgRNA Constructs

For the design of sgRNAs, the potential Sa-Cas9 binding sites were identified in silico by determining the Sa-Cas9 protospacer adjacent motif (PAM) sites, NNGRRT (R can be either G or A), within TRE promoter, human or mouse scn1a proximal promoter region, including sequences post transcription starting site. The 20 nucleotides 5′ of each PAM was cloned into a vector containing the SaCas9 trans-activating crispr RNA (tracrRNA). (Ma et al. 2018) All gRNAs-tracrRNA were driven by the U6 promoter.

### Example 6: Zinc Finger Protein Activator Constructs

Sa-dCas9 coding sequence in Sa-dCas9-TAD15 was replaced with zinc finger proteins coding sequences. In E2C-TAD15, the humanized coding sequence for the zinc finger protein, which binds to E2C site on erb2/Her2 promoter region, was synthesized according to a publication (Beerli et al. PNAS 1998), and linked to the 5′-end of TAD15 sequence. In ZFP-C-TAD15, the humanized coding sequence for ZFP-C (Zeitler et al. 2019), was synthesized and linked to the 5′-end of TAD15 sequence. Nav-ZF2, zinc finger protein targeting SCN1A gene at sequence 5′-GGC-GAG-GAT-GAA-GCC-GAG-3′ (SEQ ID NO: 90) was built into an Sp1C zinc finger framework according to (Beerli, PNAS 1998, Segal et al. PNAS 1999), was linked to the 5′-end of TAD13, TAD14 and TAD15 sequences, respectively. One nuclear localization sequences (NLS) was placed at N-terminal end of the zinc finger protein, and another one was between TAD and HA tag.

### Example 7: Cells and Cell Transfection

HEK293T cells were plated in poly-D-lysine coated 96 well black clear bottom plates at 25,000 cells per well in 100 μl of DMEM (Life Technologies, 11965092) supplemented with 10% heat-inactivated fetal bovine serum, 1×GlutaMAX (Life Technologies, 35050061) and 1× penicillin/streptomycin (Life Technologies, 10378016) at 37° C. with 5% CO2. On the following day, cultures were transfected using Lipofectamine 3000 (Life Technologies, L300015) according to the manufacturer's instructions. The amount of each plasmid per transfection sample was as follows: 20 ng of mini Sa-dCas9-VPR, 60 ng of sgRNA plasmid, 25 ng of SCN1A promoter reporter plasmid and 0.6 ng of hGPK-renilla luciferase plasmid. Two days later, luciferase activity was measured.

### Example 8: Recording of Luciferase Activity

Two days after transfection, culture media was removed and 50 ul fresh DMEM complete media was added to each well. Luciferase activity was measured using the DualGlo Luciferase Assay System (Promega, E2920) according to manufacturer's instructions. Briefly, 50 ul of the firefly luciferase substrate was added to each well and samples were mixed. After 10 minutes at room temperature, firefly luciferase activity was recorded using the Envision (Perkin Elmer). Subsequently, 50 ul of the renilla luciferase substrate was added to each well and mixed. After 10 minutes at room temperature, the renilla luciferase activity was recorded on the Envision (Perkin Elmer).

### Example 9: Lentiviral Zinc Finger Protein Activator Packaging

293T cells were plated in poly-D-lysine coated 10 cm dishes at 4,000,000 cells per dish with 10 ml culture medium. A day later, cultures were transfected lentiviral vectors carrying zinc finger protein transactivitor and helper plasmids (pMD2.G, psPAX2) using Lipofectamine 3000 (Life Technologies, L300015). Forty-six hours later, medium were collected and cell debris were removed from the medium by centrifugation at 10,000×g for 5 minutes. The viruses in the supernatants were concentrated by ultracentrifugation at 49,000×g for 90 minutes. The pellets were re-suspended in Neurobasal Plus medium (Thermo Fisher Scientific, A3582901), made into aliquots, and frozen at −80° C. until use.

### Example 10: Mouse GABAergic Neuron Culture and Neuronal Gene Activation

GABAergic neurons were dissected from medial ganglionic eminence (MGE) of embryonic day 13 mice. The MGEs were dissociated into a single cell suspension using papain dissociation system (Worthington Biochemical Corporation) according the manufacture's protocol, and cells were plated on a glial support layer on 96-well plates coated with Poly-D-Lysine in Neurobasal Plus medium at 15,000 neurons/well. At 2 or 3 day in vitro (DIV), neuronal cultures were infected lentiviruses for 4 hours. Each virus dose was chosen to achieve 70-90% infection efficiency. After infection, the cultures were washed three times in plain Neurobasal medium (Thermo Fisher Scientific), and then the conditioned medium/fresh medium (50/50) was returned to the plates and the cultures were continued to be maintained as described. At DIV7, cells were lysed and RNA were harvest. SCN1A and MAP2 transcripts were analyzed by qRT-PCR. The SCN1A level in each sample was normalized against MAP2 level of the same sample. The increase of SCN1A transcript by transcription activator is measured by fold of normalized SCN1A mRNA level over normalized SCN1A mRNA level in cells without virus.

### Example 11. Transcription Factors for Activation of TRE Promoter

TAD1, TAD2+ (TAD2 with three nuclear localization signal (NLS) sequences), TAD2-(TAD2 with two NLS sequences) and TAD3 were linked to mini Sa-Cas9 activate TRE containing promoters. Plasmid carrying sgRNA targeting TRE sequence, plasmids of mini Sa-dCas9-activator, TRE promoter-luciferase, and GPK-renilla luciferase were transfected into HEK293T cells. After two days, the luciferase activities were read. The promoter activity was calculated by normalizing firefly luciferase activity with renila luciferase activity. The relative promoter activity triggered by Sa-dCas9-activator was determined by calculating the fold change from the control guide RNA sample. 1×TRE promoter contains one TRE site; 3×TRE promoter carries three repeats of TRE site. VPR3 (Ma et al. 2018) was also linked to mini Sa-Cas9 in the same fashion, and was used a positive transcription activation factor control. The sequence of sgRNA targeting TRE is listed SEQID NO: 64.

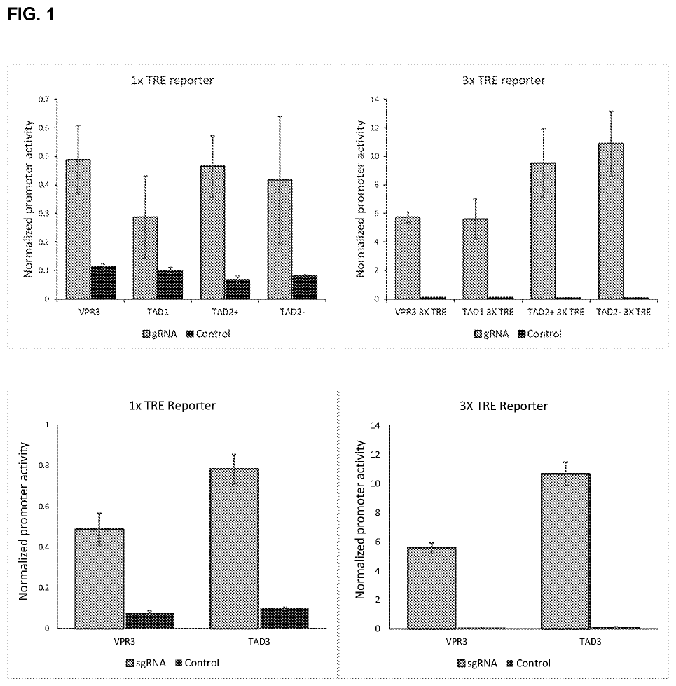

As shown in FIG. 1, TAD1, TAD2+, TAD2-show higher activity compared to VPR3 in activating TRE promoter.

### Example 12. Transcription Factors for Activation of SCN1A Promoter

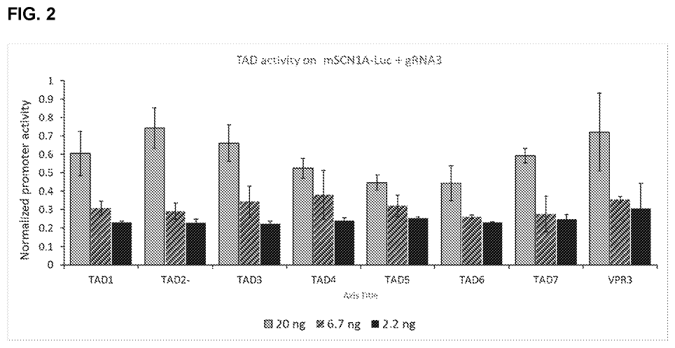

Plasmids of mini Sa-dCas9-activator (TAD1 through TAD7), mouse scn1a promoter-luciferase, gRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. Each mini Sa-dCas9-activator was transfected at 20, 6.7, and 2.2 ng/well, the results showed the dose-dependent effects. The sequence of sgRNA3 is corresponding to SEQ ID NO: 65. The results are shown in FIG. 2.

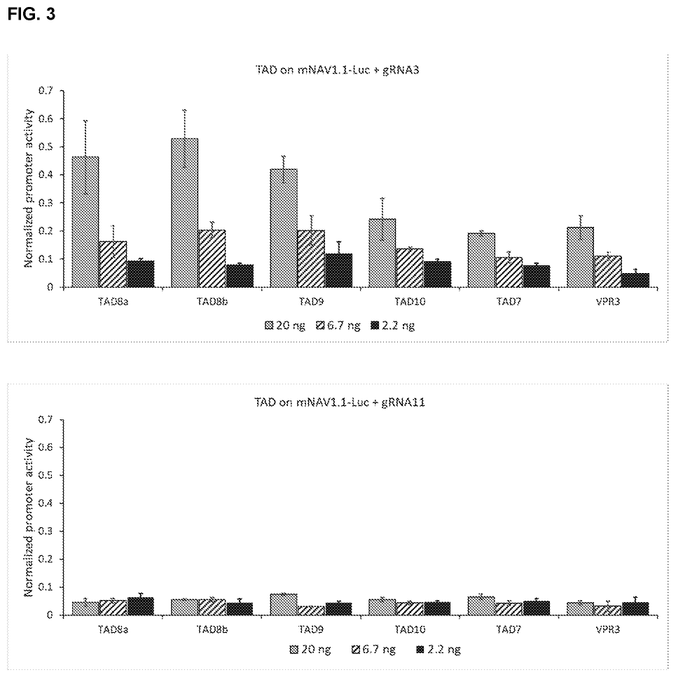

Plasmids of mini Sa-dCas9-activator (TAD7 through TAD10), mouse scn1a promoter-luciferase, gRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. Each mini Sa-dCas9-activator was transfected at 20, 6.7, and 2.2 ng/well, the results showed the dose-dependent activations when co-transfected with active guide RNA, sgRNA3. The promoter was not activated when miniSa-dCas9 was co-transfected with an inactive guide RNA, sgRNA11. The sequences of sgRNA3 and sgRNA11 are corresponding to SEQ ID NO: 65, SEQ ID NO: 66, respectively. The results are shown in FIG. 3.

### Example 13. Transcription Factors for Activation of SCN1A Promoter when Paired with sgRNA

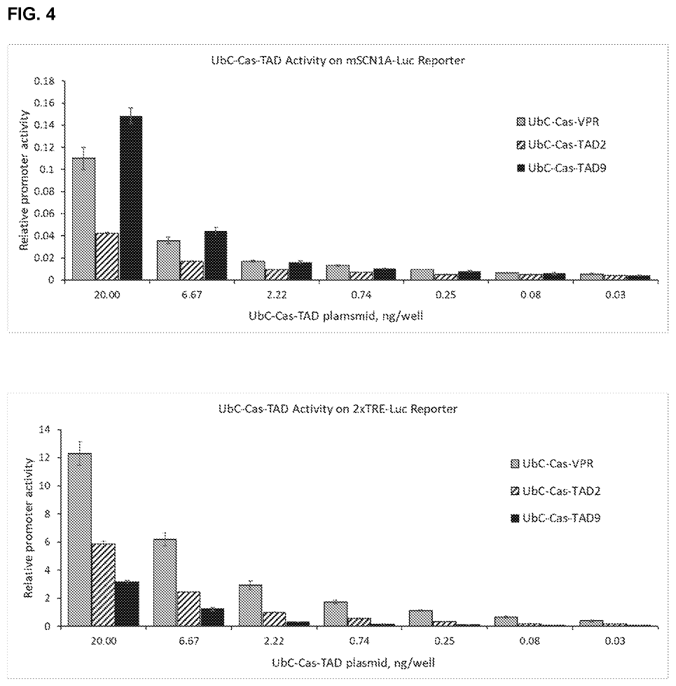

Mini Sa-dCas9-TADs driven by ubiquitin C promoter activate mouse scn1a promoter and TRE containing promoter when paired with active sgRNA. Various amount of mini Sa-dCas9-activators (TAD2 TAD9, and VPR), mouse scn1a promoter-luciferase or TRE containing promoter, TRE sgRNA for TRE containing promoter; sgRNA3 for mSCN1A promoter, respectively, and GPK-renilla luciferase were transfected into HEK293T cells. After two days, the luciferase activities were read. The promoter activity was calculated by normalizing firefly luciferase activity with renila luciferase activity. The results are shown by FIG. 4.

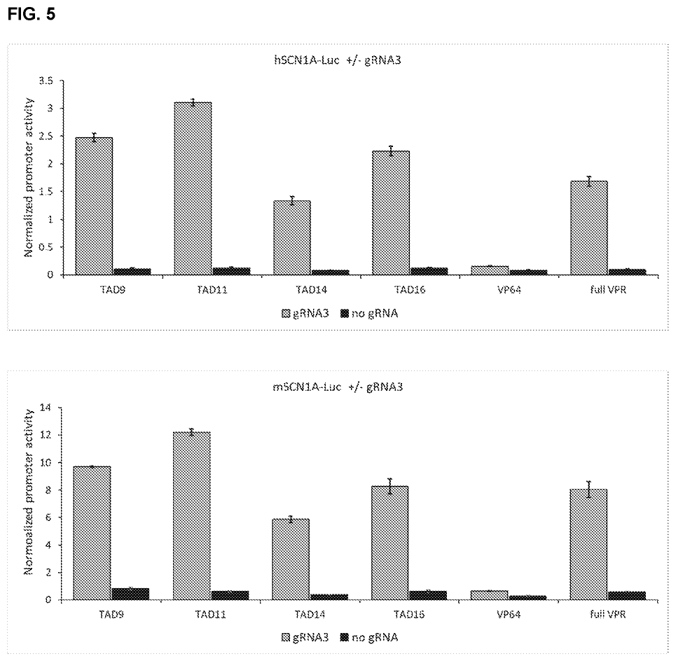

Plasmid of mini Sa-dCas9-activator, mouse scn1a promoter-luciferase or human scn1a promoter-luciferase, gRNA targeting scn1a promoter, and GPK-renilla luciferase were transfected into HEK293T cells. After two days, the luciferase activities were read. The promoter activity was calculated by normalizing firefly luciferase activity with renila luciferase activity. The newly created transcription activation domains (TAD9, TAD11, TAD14, and TAD16) have comparable or higher activity than VPR (Chavez et al. 2015), and stronger activity than VP64. The results are shown by FIG. 5.

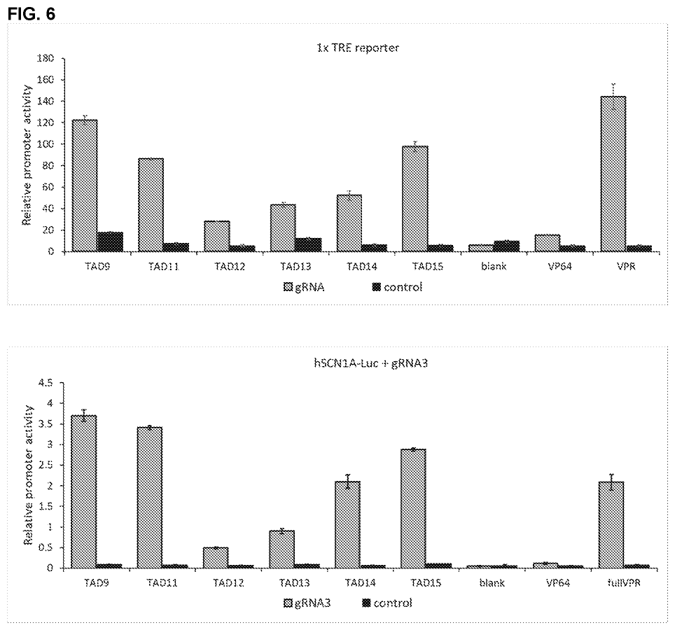

Plasmid of mini Sa-dCas9-activator, TRE containing promoter or human scn1a promoter-luciferase, gRNA targeting scn1a promoter, and GPK-renilla luciferase were transfected into HEK293T cells. After two days, the luciferase activities were read. The promoter activity was calculated by normalizing firefly luciferase activity with renila luciferase activity. The newly created transcription activation domains have comparable or higher activities than VPR (Chavez et al. 2015), and have stronger activities than VP64. FIG. 6 shows activity of TAD9, TAD11, TAD12, TAD13, TAD14, TAD15, compared to VP64 and full VPR for activation of TRE and human SCN1A promoters.

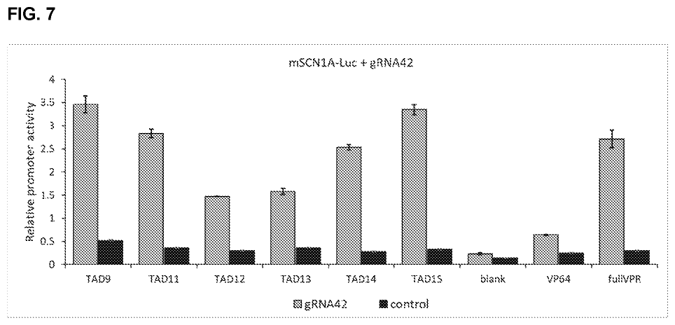

Plasmids of mini Sa-dCas9-activator, mouse scn1a promoter-luciferase, sgRNA targeting mouse scn1a promoter, and GPK-renilla luciferase were transfected into HEK293T cells. After two days, the luciferase activities were read. The promoter activity was calculated by normalizing firefly luciferase activity with renila luciferase activity. The sequences of sgRNA42 is corresponding to SEQ ID NO: 67. FIG. 7 shows activity of TAD9, TAD11, TAD12, TAD13, TAD14, TAD15, compared to VP64 and full VPR in activation of mouse SCN1A promoter with a different active sgRNA.

### Example 14. Transcription Factors for Activation of TRE Reporter Gene in U2OS Cell Line

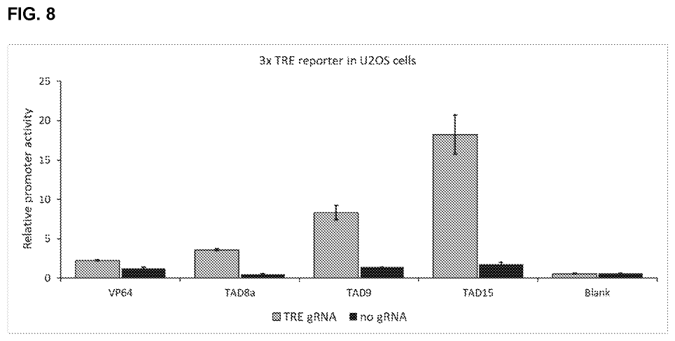

Plasmid of mini Sa-dCas9-activator, TRE containing promoter or human scn1a promoter-luciferase, gRNA targeting TRE sequence, and GPK-renilla luciferase were transfected into U2OS cells. After two days, the luciferase activities were read. The promoter activity was calculated by normalizing firefly luciferase activity with renila luciferase activity. TAD8a, TAD9 and TAD15 show higher activity than VP64, and TAD15 shows the highest activity among all, as shown in FIG. 8.

### Example 15. Activity of Transcription Factor Containing Zinc Finger Protein

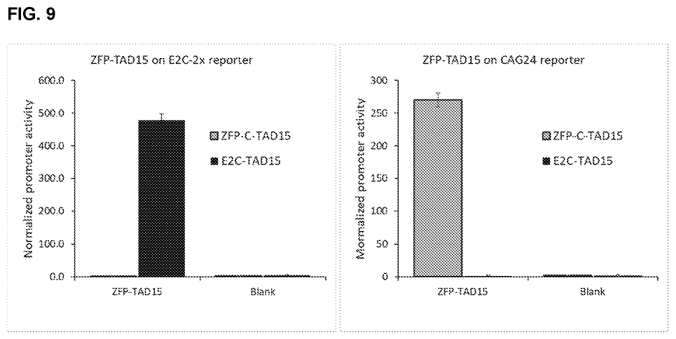

Zinc finger protein ZFP-C targeting 6 CAG repeats (SEQ ID NO: 70, “6 CAG repeats” disclosed as SEQ ID NO: 93) and zinc finger protein E2C targeting human erb2 gene promoter (SEQ ID NO: 71) were linked to TAD15. Each transcription activator were co-transfected with a reporter carrying two E2C elements (E2C 2×) or a reporter carrying 8 CAG repeats (CAG24) (SEQ ID NO: 94), and GPK-renilla luciferase were transfected into HEK293T cells. After two days, the luciferase activities were read. The promoter activity was calculated by normalizing firefly luciferase activity with renila luciferase activity. The transcription factors specifically activated promoters: E2C-TAD15 only activated E2C element containing promoter; ZFP-C-TAD15 only activated CAG repeats containing promoter. FIG. 9 shows activity of TAD15 linked to zinc finger proteins for enhancing gene transcription.

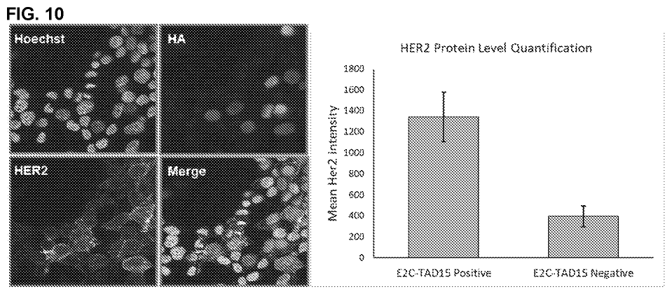

Zinc finger protein E2C (SEQ ID NO: 71) recognizes an element in human erb2 gene promoter (Beerli et al 1998). Erb2 encodes receptor protein Her2. E2C-TAD15 was tranfected into HEK293T cells. The transfected cells were identified by staining of hemagglutinin (HA), which was tagged onto E2C-TAD15. The Her2 expression levels were quantified by immunostaining of Her2 on cells surface. Cells with E2C-TAD15 (HA positive) showed more than three fold increase in Her2 level comparing to untransfected cells (HA negative). Results are shown in FIG. 10.

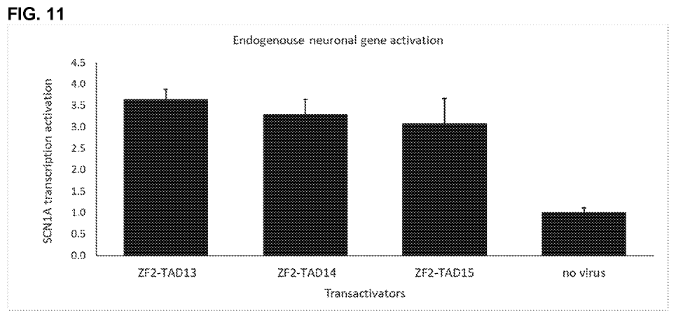

Zinc finger protein Nav-ZF2 (SEQ ID NO: 61) targeting SCN1A promoter was linked to TAD13, TAD14, and TAD15, respectively. They were packaged into lentiviruses and used to infect cultured neurons to achieve 70-90% transduction rate. Four days later, cells were lysed and RNA were harvest. SCN1A and MAP2 transcripts were analyzed by qRT-PCR. The increase of SCN1A transcript by transcription activator is measured by fold of normalized SCN1A mRNA level over normalized SCN1A mRNA level in cells without virus. Activity of TAD15 in activation of endogenous neuronal gene in mouse GABAergic neurons is shown in FIG. 11.

It is understood that the examples and aspects described herein are for illustrative purposes only and that various modifications or changes in light thereof will be suggested to persons skilled in the art and are to be included within the spirit and purview of this application and scope of the appended claims.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety (or as context dictates), to the same extent as if each were incorporated by reference individually. In case of conflict, the present specification, including definitions, will control.

What is claimed is:

1. A transcription factor comprising a DNA binding domain and at least three transcriptional activation domains (TADs), wherein the TADs may be same or different.

2. The transcription factor of claim 1, wherein the TADs comprises i) at least one acidic TAD and at least one Q-rich TAD, or ii) at least one acidic TAD and at least one P-rich TAD.

3. The transcription factor of claim 2, wherein the acidic TAD comprises one or more copies of a TAD selected from VP16, VP64, VP7, ATF6, TFE3, ATF6-11, or a fragment thereof.

4. The transcription factor of claim 2, wherein the Q-rich TAD comprises one or more copies of a TAD selected from Oct2-Q, SP1-Q1, or a fragment thereof.

5. The transcription factor of claim 2, wherein the P-rich TAD comprises one or more copies of a TAD selected from TFAP2-P, Oct2-P, or a fragment thereof.

6. The transcription factor of claim 1 or 2, wherein the TADs are selected from the group consisting of full-length VP64, one or multiple repeats of VP16, full-length or partial p65, full-length RTA, full-length VP7, full-length or partial TEF3, full-length or partial ATF6-11, full-length or partial ATF6-Acidic, full-length or partial SP1 Q-rich, full-length or partial Oct2 Q-rich, full-length or partial Oct2 P-rich, full-length or partial TFAP2 P-rich, active fragments of VP64, transcription-active fragments of p65, transcription-active fragments of RTA active fragments of VP7, active fragments of TEF3, active fragments of ATF6-11, active fragments of ATF6-Acidic, active fragments of SP1 Q-rich, active fragments of Oct2 Q-rich, active fragments of Oct2 P-rich, active fragments of TFAP2 P-rich, and any combinations thereof.

7. The transcription factor of any one of claims 1-6, wherein the total length of the TADs is less than 2000aa, less than 1500aa, less than 1000aa, less than 750aa, less than 500aa, less than 300aa, less than 250aa, less than 200aa, or less than 150aa in length.

8. The transcription factor of claim 1, wherein the transcription factor comprises a sequence having at least 95% sequence identity to any of SEQ ID NOS: 1-28, or a sequence having at least 80%, 85%, 90%, preferably 95% sequence identity with SEQ ID Nos: 1-28.

9. The transcription factor of claim 1, wherein the transcription factor comprises a sequence having any one of SEQ ID NOs: 1-18, or a sequence having at least 80%, 85%, 90%, 95%, 96%, 97%, 98%, or 99% sequence identity thereto.

10. The transcription factor of claim 1, wherein the nucleic acid sequence encoding the transcription factor comprises a sequence of SEQ ID Nos: 30-47 or a sequence having at least 80%, at least 85%, at least 90% or at least 95% sequence identity thereto.

11. The transcription factor of claim 1, wherein the DNA binding domain is linked to a transcription factor of any one of claims 1-10 with or without a linker.

12. The transcription factor of claim 11, wherein the DNA binding domain is linked to the N-terminus of the transcription factor directly or via a linker.

13. The transcription factor of claim 11, wherein the DNA binding domain is linked to the C-terminus of the transcription factor directly or via a linker.

14. The transcription factor of any one of claims 1-13, wherein the DNA binding domain binds to a genomic region of a gene of interest.

15. The transcription factor of claim 14, wherein the DNA binding domain is a gRNA/Cas complex, a transcription activator-like (TAL) effector, or a zinc finger protein.

16. The transcription factor of claim 15, wherein the gRNA/Cas complex comprises a Cas molecule that is or is derived from S. pyogenes Cas9, C. jejune Cas9, S. aureus Cas9, or Deltaproteobacteria (Dpb) CasX.

17. The transcription factor of claim 16, wherein the Cas molecule lacks one or more activities, optionally wherein the one or more activities is a cleavage activity.

18. The transcription factor of any one of claims 16-17, wherein the Cas molecule is encoded by a nucleic acid molecule comprising fewer than 4,000 nucleotides, optionally wherein the Cas molecule is encoded by a nucleic acid molecule comprising fewer than 3,500 nucleotides, or fewer than 2,500 nucleotides.

19. The transcription factor of any one of claims 16-18, wherein the Cas molecule is or is derived from S. aureus Cas9.

20. The transcription factor of claim 19, wherein the Cas molecule comprises a deletion of one or more amino acids as compared to the wild-type Cas molecule sequence.

21. The transcription factor of claim 15, wherein the DNA binding domain is a zinc finger protein.

22. The transcription factor of claim 21, wherein the DNA binding domain binds to the promoter region of the gene of interest.

23. The transcription factor of claim 22, wherein the DNA binding domain is a zinc finger protein that binds to 6, 9, 12, 15, 18, 21 or 24 bp sequence of the promoter region of the gene of interest.

24. A nucleic acid molecule encoding one or more components, optionally all of the components of the transcription factor of any one of claims 1-23.

25. A vector comprising the nucleic acid molecule of claim 24.

26. The vector of claim 25, comprising a first promoter operably linked to the nucleic acid sequence encoding the DNA binding domain.

27. The vector of claim 26, comprising a promoter that drives transcription in a human cell.

28. The vector of any one of claims 26-27, wherein the first promoter is operable in a neuron.

29. The vector of claim 28, wherein the neuron is GABAergic neuron or an inhibitory neuron, or an inhibitory interneuron.

30. The vector of claim 29, wherein the neuron is a parvalbumin-positive GABAergic neuron, or somatostatin-positive GABAergic neuron, or vasoactive intestinal peptide-positive GABAergic neuron.

31. The vector of any one of claims 25-30, further comprising one or more of:
a. a polyA sequence;
b. an intron sequence; or
C. an enhancer sequence.

32. The vector of any one of claims 25-31, further comprising a regulatory element that controls the production and/or degradation of the transcription factor.

33. The vector of claim 32, wherein the regulatory element is a minigene linked to the transcription factor, wherein the minigene comprises a splice modulator binding sequence and wherein, in the presence of a splice modulator, said minigene undergoes splicing that results in an increased or decreased expression of the transcription factor.

34. The vector of claim 32, wherein the regulatory element is a destabilizing domain, wherein in the presence of a small molecule that binds specifically to the destablizing domain, the expression of the transcription factor is decreased.

35. The vector of any one of claims 25-34, wherein the vector is an adenoviral vector, an adeno-associated viral (AAV) vector, or a lentiviral vector, or adenoviral vector, or herpes simplex viral vector.

36. The vector of claim 35, wherein the vector is an AAV vector.

37. The vector of claim 36, wherein the vector is an AAV2, AAV3, AAV4, AAV5, AAV6, AAV7, AAV8, AAV9, or AAV.rh10 vector.

38. A cell comprising the vector of any one of claims 25-37.

39. A method of selective expression of a transgene in a subject, comprising administering the viral vector of any of the claims 25-37 to the subject.

40. A method of treating a disease associated with a mutation in a gene of interest, comprising administering the viral vector of any of the claims 25-38 to a subject in need thereof.
